(19) United States
(12) Patent Application Publication
(10) Pub. No.: US 2024/0066062 A1
(43) Pub. Date: Feb. 29, 2024

(54) METHODS AND MATERIALS FOR TREATING CANCER

(71) Applicant: Mayo Foundation for Medical Education and Research, Rochester, MN (US)

(72) Inventors: Richard G. Vile, Rochester, MN (US); Laura Evgin, Vancouver (CA); Timothy J. Kottke, Oronoco, MN (US)

(21) Appl. No.: 18/266,472

(22) PCT Filed: Dec. 10, 2021
(86) PCT No.: PCT/US2021/062813

§ 371 Date: Jun. 9, 2023

Related U.S. Application Data
(60) Provisional application No. 63/275,753, filed on Nov. 4, 2021.
(60) Provisional application No. 63/149,882, filed on Feb. 16, 2021.
(60) Provisional application No. 63/124,611, filed on Dec. 11, 2020.

(51) Int. Cl. A61K 35/17; A61K 35/76; A61K 39/00; A61P 35/00; C07K 16/28; C12N 5/0783

(52) U.S. Cl. CPC A61K 35/17; A61K 35/76; A61K 39/4611; A61K 39/4631; A61K 39/464429; A61P 35/00; C07K 16/2809; C12N 5/0636; A61K 2239/13; A61K 2239/38

(57) ABSTRACT

This document provides methods and materials involved in treating cancer. For example, methods and materials for using T cells (e.g., chimeric antigen receptor (CAR) T cells) and one or more antigenic compositions (e.g., one or more compositions including one or more antigens) to treat a mammal (e.g., a human) having cancer are provided.

### CROSS-REFERENCE TO RELATED APPLICATIONS

This application claims the benefit of U.S. Patent Application Ser. No. 63/124,611, filed on Dec. 11, 2020, of U.S. Patent Application Ser. No. 63/149,882, filed on Feb. 16, 2021, and of U.S. Patent Application Ser. No. 63/275,753, filed on Nov. 4, 2021. The disclosures of the prior applications are considered part of (and are incorporated by reference in) the disclosure of this application.

### SEQUENCE LISTING

This application contains a Sequence Listing that has been submitted electronically as an ASCII text file named 07039-2005WO1_Sequence_Listing_ST25. The ASCII text file, created on Nov. 29, 2021, is 18 kilobytes in size. The material in the ASCII text file is hereby incorporated by reference in its entirety.

### BACKGROUND

### 1. Technical Field

This document relates to methods and materials involved in treating cancer. For example, this document provides methods and materials for using T cells (e.g., chimeric antigen receptor (CAR) T cells) and one or more antigenic compositions (e.g., one or more compositions including one or more antigens) for treating a mammal (e.g., a human) having cancer.

### 2. Background Information

The rather modest efficacy of chimeric antigen receptor (CAR) T cells against solid tumors derive from multiple immune suppressive mechanisms in the tumor microenvironment, which restrict CAR T cell infiltration, persistence, and function (Newick et al., Annu. Rev. Med., 68:139-152 (2017); Schmidts et al., Front. Immunol., 9:2593 (2018); Morgan et al., Front. Immunol., 9:2493 (2018); and Labanieh et al., Nat. Biomed Eng., 2(6):377-391 (2018)).

### SUMMARY

This document provides methods and materials involved in treating cancer. For example, this document provides methods and materials for using T cells (e.g., CAR⁺ T cells) and one or more antigenic compositions (e.g., one or more compositions including one or more antigens) for treating a mammal (e.g., a human) having cancer. In some cases, a mammal (e.g., a human such as a human having cancer) can be administered (a) a population of different T cells engineered to each include a CAR and (b) an antigenic composition (e.g., a composition including one or more viruses such as one or more oncolytic viruses, a composition including one or more viruses designed to express one or more antigens of interest, and/or a composition including one or more antigenic polypeptides of interest) to stimulate in vivo generation of memory T cells specific for one or more of the antigen(s) of the antigenic composition via the endogenous T cell receptors (TCRs), with at least some of those generated memory T cells also expressing the CAR. For example, a mammal can be administered (a) a population of different T cells that each express a particular CAR in addition to their endogenous TCR and (b) an antigenic composition to stimulate in vivo generation of memory T cells from at least a few CAR⁺ T cells of that administered population. It will be appreciated that the mammal's natural T cell population (endogenous T cells) will include some members of the repertoire that also will be stimulated via the antigenic composition, but those cells will only include the endogenous TCR and not the CAR. Those memory T cells generated from the population of CAR⁺ T cells administered to the mammal can have the ability to direct an immune response (e.g., expand to form populations of effector T cells) against a target via either the CAR or the endogenous TCR (e.g., an endogenous TCR specific for an antigen of the antigenic composition) of that memory T cell. These generated memory T cells within the mammal can be quickly and effectively stimulated to generate populations of effector T cells that target the targets of the CAR by subsequently administering one or more of the antigens recognized by the endogenous TCR of those CAR⁺ memory T cells.

As demonstrated herein, co-administration of (a) a population of CAR⁺ T cells each with its own endogenous TCR and (b) an oncolytic virus to a mammal can stimulate in vivo generation of dual-specific tissue-resident memory (T_{RM}) T cells (e.g., T_{RM} CAR⁺ T cells) that can recognize either the target of the CAR (e.g., a cancer cell) via the CAR or a T cell epitope of the oncolytic virus via an endogenous TCR specific for that T cell epitope. Also as demonstrated herein, the dual-specific T_{RM} CAR⁺ T cells generated within a mammal can be reactivated to direct immune responses (e.g., populations of effector T cells) against the target of the CAR (e.g., cancer) by administering a boost of the oncolytic virus (or an antigenic portion thereof) to the mammal. In such cases, the boost can stimulate the memory T cells via their endogenous TCR that is specific for the oncolytic virus, and they can be free to hunt and kill and/or to generate effector T cells that can hunt and kill the CAR targets via their provided CAR.

The ability to generate memory T cells and/or effector T cells in a mammal as described herein (e.g., by administering a population of different T cells expressing one or more CARs and one or more antigenic compositions) provides a unique opportunity to use immunotherapy to target (e.g., to locate and destroy) cancer cells, including cancer cells in solid tumors, which can be undetectable by the immune system, and cancer cells at secondary (e.g., metastatic) locations. For example, the dual-specific memory T cells can be more active against cancer cells, can persist longer in vivo than conventional CAR⁺ T cells used in current immunotherapies, and can be rapidly re-activated in vivo to generate CAR⁺ effector T cells via a subsequent administration of a boosting antigen, thereby resulting in long-term tumor control.

In general, one aspect of this document features a method for treating a mammal having cancer. The method comprises (or consists essentially of or consists of) (a) administering a population of T cells with different endogenous T cell receptors (TCRs) to the mammal, wherein the T cells comprise a chimeric antigen receptor (CAR) that targets the cancer; (b) administering a first antigenic composition to the mammal, wherein at least some of the T cells of the population form memory T cells within the mammal, wherein the memory T cells comprise the CAR and an endogenous TCR specific for an antigen of the first antigenic composition; and (c) administering a second antigenic composition comprising the antigen to the mammal, wherein the memory T cells are stimulated via their endogenous TCRs to form effector T cells comprising the CAR, and wherein the effector T cells reduce the number of cancer cells within the mammal. The mammal can be a human. The cancer can be selected from the group consisting of brain stem gliomas, pancreatic cancers, bile duct cancers, lung cancers, skin cancers, prostate cancers, breast cancers, ovarian cancers, liver cancers, colorectal cancers, germ cell tumors, hepatocellular carcinoma, bowel cancers, multiple myeloma, lymphomas, and leukemias. The population of T cells with different endogenous TCRs can comprise naïve T cells. The naïve T cells can be selected from the group consisting of CD4⁺ T cells, CD8⁺ T cells, and any combination thereof. The CAR can target a tumor-specific antigen on the cancer. The tumor-specific antigen can be selected from the group consisting of cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2. The first antigenic composition can comprise a virus. The virus can be an oncolytic virus. The virus can be selected from group consisting of a vesiculovirus, a Maraba virus, a reovirus, adenoviruses, vaccinia viruses, Newcastle disease viruses, polioviruses, HSV viruses, and measles viruses. The endogenous TCR specific for the antigen can be an endogenous TCR specific for an antigen of the virus. The first antigenic composition can comprise a virus expressing an antigen exogenous to the virus. The endogenous TCR specific for the antigen can be an endogenous TCR specific for the antigen exogenous to the virus. The first antigenic composition can comprise an antigenic polypeptide. The endogenous TCR specific for the antigen can be an endogenous TCR specific for the antigenic polypeptide. The population of T cells with different endogenous TCRs and the first antigenic composition can be administered to the mammal within from about 1 second to about 48 hours of each other. The population of T cells with different endogenous TCRs and the first antigenic composition can be administered to the mammal at the same time. The population of T cells with different endogenous TCRs and the first antigenic composition can be administered to the mammal are as a single composition. The memory T cells can be CD69⁺ and CD103⁺. The memory T cells can be selected from the group consisting of central memory T cells (T_{CM} cells), effector memory T cells (T_{EM} cells), terminally differentiated effector memory T cells (T_{EMRA} cells), and tissue resident memory T cells (T_{RM}). The second antigenic composition can be administered to the mammal at least 5 days after the administering of the population of T cells and the administering of the first antigenic composition. The cancer cells within the mammal can be reduced by at least 25 percent. The method can be effective to improve survival of the mammal. The survival of the mammal can be improved by at least 25 percent.

In another aspect, this document features a method for generating memory T cells within a mammal. The method comprises (or consists essentially of or consists of) (a) administering a population of T cells with different endogenous T cell receptors (TCRs) to the mammal, wherein the T cells comprise a chimeric antigen receptor (CAR); and (b) administering an antigenic composition to the mammal, wherein at least some of the T cells of the population form memory T cells within the mammal, wherein the memory T cells comprise the CAR and an endogenous TCR specific for an antigen of the antigenic composition. The mammal can be a human. The population and the antigenic composition can be administered to the mammal as a single composition. The CAR can target a tumor-specific antigen. The tumor-specific antigen can be selected from the group consisting of cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2. The antigenic composition can comprise a virus. The virus can be an oncolytic virus. The virus can be selected from group consisting of a vesiculovirus, a Maraba virus, a reovirus, adenoviruses, vaccinia viruses, Newcastle disease viruses, polioviruses, HSV viruses, and measles viruses. The endogenous TCR specific for the antigen can be an endogenous TCR specific for an antigen of the virus.

The antigenic composition can comprises a virus expressing an antigen exogenous to the virus. The endogenous TCR specific for the antigen can be an endogenous TCR specific for the antigen exogenous to the virus. The antigenic composition can comprise an antigenic polypeptide. The endogenous TCR specific for the antigen can be an endogenous TCR specific for the antigenic polypeptide. The mammal can have cancer. The cancer can be selected from the group consisting of brain stem gliomas, pancreatic cancers, bile duct cancers, lung cancers, skin cancers, prostate cancers, breast cancers, ovarian cancers, liver cancers, colorectal cancers, germ cell tumors, hepatocellular carcinoma, bowel cancers, multiple myeloma, lymphomas, and leukemias. The memory T cells can be selected from the group consisting of central memory T cells (T_{CM} cells), effector memory T cells (T_{EM} cells), terminally differentiated effector memory T cells (T_{EMRA} cells), and tissue resident memory T cells (T_{RM}). The administering can comprise intravenous injection.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic of an exemplary therapeutic framework for in vivo selected dual-specific CAR⁺ T cells for cancer immunotherapy. CAR⁺ T cells specific for a cancer specific target antigen can be loaded ex vivo with an oncolytic virus. Upon adoptive transfer (1), virus loaded CAR⁺ T cells can traffic to the tumor. Once there, they both kill tumor cells directly via engagement through the CAR and release their virus load. This leads to tumor cell infection and oncolysis by the virus. Simultaneously, the CAR⁺ T cells loaded with oncolytic virus can release virus, which can be processed in the lymph nodes by antigen presenting cells (2). A small fraction of the CAR⁺ T cells can express an endogenous TCR that can be activated by one or more viral antigens presented by MHC molecules to the CAR⁺ T cells. This can lead to a highly preferential expansion of the virus-specific CAR⁺ T cell in vivo (3), possibly through synergy between strong endogenous TCR signaling and CAR signaling within the T cells. These expanding populations of virus-specific CAR⁺ T cells can kill additional numbers of tumor cells (4) through both the CAR target recognition and, possibly, through recognition of virus antigens expressed by the infected tumor cells from (1). In addition, following the expansion of virus-specific CAR⁺ T cells and tumor cell killing, a proportion of the dual specific CAR⁺ T cells can differentiate into central memory (CM) and/or tissue resident memory CAR⁺ T cells (5). Upon a later exposure to the appropriate virus (or one or more viral antigens) (6), these CAR⁺ memory T cells (e.g., CAR⁺ T_{RM} cells) can rapidly respond, proliferate, and initiate a further round of tumor cell killing (7).

FIG. 2 is a graph plotting the percent of CAR⁺ T cells of total CD8⁺ T cells from spleen or blood of mice treated as indicated. The proportion of total CD8⁺ T cells in the spleen and the blood that were Thy1.1+ve (CAR T marker) and/or VSV specific (VSV tetramer +ve) at day 160 is shown in mice that received either CAR⁺ T cells and VSV-IFNβ or in mice that received CAR⁺ T cells and PBS.

FIGS. 3A-C. Mice were treated according to the schedule in FIG. 3A. FIG. 3B: CD8⁺ CAR⁺ T cells recovered from the spleens of mice treated with either PBS or with VSV-IFNβ were incubated with target B16EGFRvIII (labelled by Cell Trace Violet) and non-target B16 cells (labeled with CFSE) at an Effector:Target ratio of 2:1:1. Increased target cell specific CAR⁺ T cell killing (percent specific killing) is indicated by loss of the CTV+ve target cells compared to the non-target CFSE cell population. FIG. 3C: CAR⁺ T cells from treated groups that were either positive (dual specific) or −ve for VSV tetramer were stimulated in vitro with nothing, the VSV immunodominant peptide N52-59, B16 cells (non CAR Targets), or B16EGFRvIII cells (CAR targets). IFN-7 and degranulation marker CD107a were measured as a marker of T cell activation. Levels of CD107a, IFN-γ, TNF-α, and IL-2 produced by VSV tetramer positive or negative CD8⁺ CAR⁺ T cells from mice treated with PBS (no virus) or VSV-IFNβ are shown along with the proportion of CAR⁺ T cells that expressed 0, 1, 2, 3, or 4 of these markers of T cell activation. These data show that dual specific CAR⁺ T cells (CD8+, Thy1.1⁺, VSV tetramer positive) kill target cells more potently and express significantly higher levels of T cell activation markers than CAR⁺ T that are not dual specific.

FIG. 4 contains amino acid sequences (SEQ ID NOs:1-4) for the indicated exemplary CARs.

FIG. 5 is a graph plotting mouse survival time. Mice with 8 day brainstem CT2A-EGFRvIII tumors were treated on day 8 with PBS or with 10⁷ anti-EGFRvIII CAR T cells. The CAR T cells were left unloaded or were loaded in vitro with VSV-IFNβ (MOI 1.0, 4° C. for 1 hour). On day 15 mice were boosted with PBS or with 10⁷ plaque-forming unit (pfu) of either VSV-IFNβ or reovirus.

FIG. 6 is a graph plotting mouse survival time. Mice with 8 day subcutaneous B16-EGFRvIII tumors were treated on day 8 with PBS, with 10⁷ pfu of Ad-ova, or with 10⁷ anti-EGFRvIII CAR T cells. The CAR T cells were left unloaded, or were loaded in vitro with Ad-ova (MOI 1.0, 4° C. for 1 hour). On day 19 mice were boosted with PBS or with 10⁷ pfu of either Ad-ova, VSV-GFP, VSV-ova, or Ad-GFP.

FIG. 7 is a graph plotting mouse survival time. Mice with 8 day subcutaneous B16-EGFRvIII tumors were treated on day 8 with PBS, with 10⁷ pfu of VSV-GFP, or with 10⁷ anti-EGFRvIII CAR T cells. The CAR T cells were left unloaded, or were loaded in vitro with VSV-ova or VSV-GFP (MOI 1.0, 4° C. for 1 hour). On day 19 mice were boosted with PBS or with 10⁷ pfu of either Ad-ova or VSV-ova.

FIGS. 8A-8B. Mice were treated according to the schedule in FIG. 8A. On day −14, mice were administered PBS, 10⁷ pfu VSV-ova, or 10⁷ anti-EGFRvIII CAR T cells. The CAR T cells were left unloaded, or were loaded in vitro with VSV-ova (MOI 1.0, 4° C. for 1 hour). On day 0, mice were injected subcutaneously with 2×10⁵ B16-EGFRvIII tumor cells. On day 20 mice were boosted with PBS or with 10⁷ pfu of either Ad-GFP, Ad-ova or VSV-ova. FIG. 8B: Graphs plotting tumor size.

FIG. 9 is a graph plotting mouse survival time. On day −14, mice were administered PBS or 10⁷ anti-EGFRvIII CAR T cells. The CAR T cells were left unloaded, or were loaded in vitro with VSV-ova (MOI 1.0, 4° C. for 1 hour). On day 0, mice were injected intra-cranially into the brainstem with 2×10⁴ CT2A-EGFRvIII tumor cells. On day 7 mice were boosted with PBS or with 10⁷ pfu of either Ad-GFP, Ad-ova, or VSV-ova.

FIG. 10 contains graphs plotting tumor size of recurrent tumors. Mice with 8 day subcutaneous B16-EGFRvIII tumors were treated on day 8 with 10⁶ anti-EGFRvIII CAR T cells loaded in vitro with reovirus (MOI 1.0, 4° C. for 1 hour). On day 15 mice were boosted with 10⁷ pfu reovirus. Out of a group of 15 mice, 3 developed recurrent tumors. Once a mouse developed a palpable recurrence, it was administered an i.v. injection of 10⁷ pfu of reovirus, VSV-GFP or PBS (arrow).

FIG. 11 is a graph plotting mouse survival time. Mice with 8 day subcutaneous B16-EGFRvIII tumors were treated on day 8 with PBS, with 10⁷ pfu of reovirus or with 10⁷ anti-EGFRvIII CAR T cells. The CAR T cells were left unloaded, or were loaded in vitro with reovirus (MOI 1.0, 4° C. for 1 hour). On day 15 mice were boosted with PBS or with 10⁷ pfu of either VSV-GFP or reovirus.

FIG. 12 contains graphs showing persistence of tissue resident memory T cells (T_{RM}). Mice treated with skin/tumor samples were excised from the mice treated as in FIG. 11. RNA was extracted and levels of expression of the markers FABP4 and FABP5 were measured by qrtPCR as an indication of levels of T_{RM} and levels of the CAR specific retroviral vector were measured as an indication of levels of CAR T at the tumor site.

FIGS. 13A-13F. Graphs showing persistence of CAR T cells. Mice with 8 day subcutaneous B16-EGFRvIII tumors were treated on day 8 with PBS, with 10⁷ pfu of reovirus or with 10⁷ anti-EGFRvIII CAR T cells. The CAR T cells were left unloaded, or were loaded in vitro with reovirus (MOI 1.0, 4° C. for 1 hour). On day 15 mice were boosted with PBS or with 10⁷ pfu of either VSV-GFP or reovirus. At the time of euthanasia due to tumor size splenocytes were harvested from 3 mice per treatment group and pooled together. CD8+(FIG. 13A) and CD4+(FIG. 13B) CAR T cells were measured by flow cytometry by measuring CD8⁺ Thy1.1⁺ or CD4⁺ Thy1.1⁺ cells. (Thy1.1 is a marker expressed within the CAR vector). FIG. 13C-13F. In addition, the pooled splenocytes from each of the treatment groups were co-cultured with target B16 (FIG. 13C), B16-EGFRvIII (FIG. 13D), CT2A-EGFRvIII (FIG. 13E), or CT2A (FIG. 13F) tumor cells at an Effector:Target ratio of 10:1. 72 hours later levels of IFN-7 in the supernatants were measured by ELISA.

FIG. 14 graphs showing persistence of CAR T cells. Mice with 8 day subcutaneous B16-EGFRvIII tumors were treated on day 8 with PBS, with 10⁷ pfu of VSV-GFP or with 10⁷ anti-EGFRvIII CAR T cells. The CAR T cells were left unloaded, or were loaded in vitro with VSV-GFP or VSV-ova (MOI 1.0, 4° C. for 1 hour). On day 15 mice were boosted with PBS or with 10⁷ pfu of either Ad-ova, or VSV-ova. At the time of euthanasia due to tumor size splenocytes were harvested from 3 mice per treatment group and pooled together. Single specific CAR (Thy1.1+ve), single specific OVA (SIINFEKL tetramer (SEQ ID NO:5) +ve) and dual specific Thy1.1+, SIINFEKL tetramer (SEQ ID NO:5)+ve CD8⁺ T cells were measured by flow cytometry (Thy1.1 is a marker expressed within the CAR vector; ova-specific CD8⁺ T cells are SIINFEKL tetramer (SEQ ID NO:5)+ve).

FIG. 15. Graphs showing infiltration of CAR T cells into the brain. Mice with 8 day brainstem CT2A-EGFRvIII tumors were treated on day 8 with PBS or with 10⁷ anti-EGFRvIII CAR T cells. The CAR T cells were left unloaded, or were loaded in vitro with Reovirus (MOI 1.0, 4° C. for 1 hour). On day 15 mice were boosted with PBS or with 10⁷ pfu Reovirus. At the time of euthanasia brains were harvested from 3 mice per treatment group and pooled together. CAR (Thy1.1+ve), CD8⁺ T cells were measured by flow cytometry.

FIG. 16 is a graph plotting mouse survival time. Mice with 8 day subcutaneous B16-CD19 tumors were treated on day 8 with PBS, with 10⁷ pfu of VSV-IFNβ, or with 10⁷ anti-CD19 CAR T cells. The CAR T cells were left unloaded, or were loaded in vitro with VSV-IFNβ (MOI 1.0, 4° C. for 1 hour). On day 15 mice were boosted with PBS or with 10⁷ pfu of either Ad-GFP or VSV-IFNβ.

FIG. 17 is a graph plotting mouse survival time. Mice with 8 day frontal lobe CT2A-EGFRvIII tumors were treated on day 8 and boosted on day 15 with: 1. PBS/PBS; 2.CAR T/PBS; 3. reovirus/PBS; 4. CAR T loaded with reovirus/PBS; 5. CAR T loaded with reovirus/reovirus; or 6. CAR T loaded with reovirus/VSV. Survival at day 90 is shown.

FIGS. 18A-18H. CAR T cells with TCR reactivity to a VSV immunodominant epitope expand following infection. (FIG. 18A) Mice bearing subcutaneous B16EGFRvIII tumors were lymphodepleted on day 3 with 5 Gy total body radiation, and treated on day 4 with 10⁷ EGFRvIII CAR T cells and on day 9 with PBS or with VSV-mIFNβ intravenously (IV) (10⁷ pfu) or intratumorally (IT) (5×10⁷ pfu). On day 16, CD8⁺ CAR T cells (identified by Thy1.1 expression) with TCR reactivity to the VSV N₅₂₋₅₉ H2-K^{b} immunodominant epitope were enumerated in the spleen and tumor. n=4 mice/group. (FIG. 18B) Representative flow plots for the CD8⁺ gate are shown for individual animals from each treatment. (FIG. 18C) The composition of the CD8 compartment for individual animals is represented in the inner rings and the average group composition in the outer ring. (FIG. 18D) The percent CD8 CAR T tetramer positive populations from FIG. 18A is represented as the mean±SD. Each symbol represents a mouse. In separate cohorts of animals, blood was collected by submandibular vein bleeds on days 17 and 32 and dual specific CAR T cells enumerated. n=3-10 mice/group. P values were determined using a one-way ANOVA with a Tukey multiple comparisons post-test. (FIG. 18E) Interim blood and experimental endpoint spleens from two independent tumor cured animals were stained for CD8, Thy1.1, and the VSV N₅₂₋₅₉ H2-K^{b} tetramer. Flow plots show the CD8⁺ Thy1.1⁺ gate. (FIG. 18F) TCR Vβ distribution as measured by flow cytometry of the CD8 CAR T cells prepared for injection, and the CD8 CAR T recovered from mice on day 16 which also received PBS, or VSV-mIFNβ intravenously or intratumorally as in FIG. 18A. n=4 mice/group. (FIG. 18G) Comparison of the TCRvβ chain usage between the input CAR T cells adoptively transferred into mice and those recovered from recipient mice reveals positive and negative selection for particular chains. (FIG. 18H) Comparison between TCRvβ chain usage in the endogenous CD8 population versus the CD8 CAR T population for each animal. For FIGS. 18G and 18H, each symbol represents a mouse.

FIGS. 19A-19G. Dual specific CAR T cells have improved function against B16EGFRvIII target cells and acquire a memory distinct phenotype. (FIG. 19A) Mice bearing subcutaneous B16EGFRvIII tumors were lymphodepleted on day 3 with 5 Gy total body radiation, and treated on day 4 with 10⁷ EGFRvIII CAR T cells and on day 9 with PBS or with VSV-mIFNβ intravenously (IV) (10⁷ pfu). On day 16, Thy1.1⁺ CD4⁻ cells were sorted by FACS. (FIG. 19B) Sorted cells were co-cultured with CTV labelled B16EGFRvIII target cells and CFSE labelled B16 non-target cells in a ratio of 2:1:1 (Effector:target:non-target). Representative flow plots gated on live Thy1.1⁻ cells are shown in left panel. In the right panel, the percent specific killing of target cells is represented as the mean±SD. Each symbol represents a mouse. The p value was determined using an unpaired two tailed t-test. (FIG. 19C) Splenocytes from FIG. 19A were left unstimulated or stimulated with the VSV N52-59 peptide. Sorted Thy1.1⁺ CD4⁻ cells were co-cultured with B16 parental or B16EGFRvIII cells at an effector:target ratio of (2:1) for 6 hours in the presence of Brefeldin A and monensin. Cells were stained with CD107a during the co-culture and the VSV N tetramer and for cytokine production following the co-culture. Representative flow plots show degranulation (CD107a) and cytokine (IFNγ) production. (FIG. 19D) The full intracellular cytokine panel is shown for all mice stimulated as described in FIG. 19C. Each symbol represents a mouse, and lines connect the unstimulated and stimulated conditions for each animal. n=4 mice/group. (FIG. 19E) Group means of Boolean gating demonstrating co-expression of cytokines and degranulation as in FIG. 19D. (FIG. 19F) CD8 CAR T cells from the spleens of mice treated as in FIG. 19A were assayed for expression of KLRG1, CD127, and CD62L. Representative flow plots gated on CD8⁺ Thy1.1⁺ and indicated as tetramer positive or negative. (FIG. 19G) Percent expression of CD62L, CD127, and KLRG1 as in FIG. 19F is represented as the mean±SD. Each symbol represents a mouse. n=4-6 mice/group. P values were determined using a one-way ANOVA with a Tukey multiple comparisons post-test.

FIGS. 20A-20F. In vitro virus loading promotes dual specific CAR T cell generation with improved therapeutic efficacy. (FIG. 20A) Mice bearing subcutaneous B16EGFRvIII tumors were treated with 10⁷ pfu VSV-GFP IV or with 10⁷ EGFRvIII CAR T cells loaded with VSV-GFP (MOI 1) at 4° C. for 1 hour [(CAR(VSV) IV]. Two days later, lymph nodes (LN) and tumors were harvested and dissociated, and virus was titered on BHK cells. n=3 mice/group. The p value was determined using an unpaired two tailed t-test on log-transformed data. (FIG. 20B) Mice bearing subcutaneous B16EGFRvIII tumors were treated with 10⁷ EGFRvIII CAR T cells either left unloaded or loaded with VSV-GFP (MOI 1); LPS; polyIC; CpG; or reovirus (MOI 1) at 4° C. for 1 hour. Two days later, lymph nodes and tumors were harvested and dissociated, and the number of Thy 1.1⁺ CD8⁺ CAR T cells enumerated. n=3 mice/group. P values were determined using a one-way ANOVA with a Tukey multiple comparisons post-test using log-transformed data. (FIG. 20C) Non tumor bearing mice were treated with 10⁷ EGFRvIII CAR T cells either unloaded, or loaded with VSV-GFP (MOI 1) at 4° C. for 1 hour [(CAR(VSV) IV]. A third group treated with unloaded CAR T cells was subsequently treated with 10⁷ pfu VSV-GFP 4 days later. Two weeks post adoptive cell transfer, Thy1.1⁺ CD8⁺ CAR T cells were isolated from spleens, and 10⁷ cells were co-cultured with B16EGFRvIII or B16 cells pre-treated for 24 hours with IFNγ; or with murine in vitro matured dendritic cells pre-loaded for 24 hours with VSV-N₅₂₋₅₉ or OVA-derived SIINFEKL peptide at an E:T ratio of 10:1. 48 hours later, levels of IFNγ were measured by ELISA. P values were determined using a one-way ANOVA with a Tukey multiple comparisons post-test using log-transformed data. (FIG. 20D and FIG. 20E) Mice bearing subcutaneous B16EGFRvIII tumors were treated on day 8 with either PBS, 10⁷ EGFRvIII CAR T cells, 10⁷ pfu VSV-mIFNβ, or with 10⁷ anti-EGFRvIII CAR T cells loaded with VSV-mIFNβ (MOI 1) at 4° C. for 1 hour [CAR(VSV)] IV. On day 15, mice were given an IV boost with PBS, with 10⁷ pfu VSV-IFNβ, or with 10⁸ pfu of a replication incompetent adenovirus Ad-OVA. Survival of mice is shown. n=7-8 mice/group. A Log Rank Mantel-Cox test was performed, and with a multiple comparison Bonferonni correction, statistical significance was set at p=0.0083. (FIG. 20F) Thy1.1⁺ CD8⁺ CAR T cells were isolated from spleens of mice in the experiment of FIG. 20E either at time of euthanasia due to tumor size, or at the end of the experiment (day 60). 2.5×10⁵ were co-cultured with live B16-EGFRvIII or B16 cells pre-treated for 24 hours with IFNγ; or with murine in vitro matured dendritic cells pre-loaded for 24 hours with VSV-N₅₂₋₅₉ or OVA-derived SIINFEKL (SEQ ID NO:5) peptide at an E:T ratio of 10:1. 48 hours later, levels of IFNγ were measured by ELISA as shown. n=3 mice/group. P values were determined using a one-way ANOVA with a Tukey multiple comparisons post-test. For FIGS. 20A, 20B, 20C, and 20F, the group mean is represented±SD. Each symbol represents a mouse.

FIGS. 21A-21G. Reovirus-loaded CAR T cells are therapeutic in multiple tumor models. (FIG. 21A and FIG. 21B) Mice bearing subcutaneous B16EGFRvIII tumors were treated on day 7 with either PBS, 10⁷ pfu Reovirus, or 10⁷ anti-EGFRvIII CAR T cells (left unloaded, or loaded in vitro with Reovirus (MOI 1.0, 4° C., 1 hour)). On day 15, mice were boosted with PBS or 10⁷ pfu VSV-GFP or Reovirus. Survival of mice is shown. n=7 mice/group. A Log Rank Mantel-Cox test was performed, and with a multiple comparison Bonferonni correction, statistical significance was set at p=0.017. (FIG. 21C) Splenocytes from mice treated in FIG. 21B at euthanasia were pooled (n=3 mice/group), and flow plots gated on the CD8⁺ population show the percent Thy1.1⁺. (FIG. 21D and FIG. 21E) Mice bearing brainstem CT2AEGFRvIII tumors were treated on day 7 with PBS, 10⁷ pfu Reovirus, or 10⁷ anti-EGFRvIII CAR T cells (left unloaded, or loaded in vitro with Reovirus (MOI 1.0, 4° C., 1 hour)). On day 15, mice were boosted with PBS or 10⁷ pfu VSV-GFP or Reovirus. A Log Rank Mantel-Cox test was performed, and with a multiple comparison Bonferonni correction, statistical significance was set at p=0.0125. (FIG. 21F) A separate cohort of animals treated as in FIG. 21D (n=3 mice/group) were euthanized on day 25, and the brains harvested. Flow plots gated on the CD8⁺ population show the percent Thy1.1⁺ from the pooled groups. (FIG. 21G) Splenocytes from FIG. 21E were co-cultured with CT2AEGFRvIII cells or CT2A parental cells at an effector to target ratio of 5:1 for 48 hours. IFNγ secretion was measured by ELISA. n=3 mice/group. P values were determined using a one-way ANOVA with a Tukey multiple comparisons post-test using log-transformed data. The group mean is represented±SD. Each symbol represents a mouse.

FIGS. 22A-22F. Dual specific CAR T cells can be expanded with TCR specificity for a virus encoded antigens. (FIG. 22A and FIG. 22B) Mice bearing subcutaneous B16EGFRvIII tumors were treated on day 8 with either PBS, 10⁸ pfu Ad-OVA, or 10⁷ anti-EGFRvIII CAR T cells (left unloaded, or loaded in vitro with Ad-OVA (MOI 1) at 4° C. for 1 hour [CAR(Ad-OVA)]. On day 15, mice were boosted with PBS or with 10⁷ pfu of Ad-GFP, VSV-GFP, or VSV-OVA. Survival of mice is shown. n=7-8 mice/group. A Log Rank Mantel-Cox test was performed, and with a multiple comparison Bonferonni correction, statistical significance was set at p=0.017. (FIG. 22C) Splenocytes from representative animals were isolated from mice in the experiment of FIG. 22B either at time of euthanasia due to tumor size, or at the end of the experiment (day 62). Flow plots gated on the CD8⁺ population show the percent Thy1.1⁺ and SIINFEKL (SEQ ID NO:5) tetramer⁺ populations. (FIG. 22D and FIG. 22E) Mice bearing subcutaneous B16EGFRvIII tumors were treated on day 8 with either PBS, 10⁷ pfu EGFRvIII CAR T cells, or 10⁷ pfu VSV-hgp100, or with 10⁷ anti-EGFRvIII CAR T cells loaded with VSV-hgp100 (MOI 1) at 4° C. for 1 hour [CAR(VSV-hGP100)]. On day 15, mice were given an intravenous boost with PBS, 10⁸ pfu Ad-GFP, or Ad-hGP100. n=7-8 mice/group. A Log Rank Mantel-Cox test was performed, and with a multiple comparison Bonferonni correction, statistical significance was set at p=0.017. (FIG. 22F) At the time of euthanasia due to tumor size or at the conclusion of the experiment (d95), Thy1.1l CD8⁺ CAR T cells were isolated from spleens, and 10⁵ cells were co-cultured with B16-EGFRvIII or B16 cells pre-treated for 24 hours with IFNγ, or with murine in vitro matured dendritic cells pre-loaded for 24 hours with the hGP100₂₅₋₃₃ H-2D^{b}-restricted peptide or the OVA-derived SIINFEKL (SEQ ID NO:5) peptide (5 μg/mL) at an E:T ratio of 10:1. 48 hours later, levels of IFNγ were measured by ELISA as shown. P values were determined using a one-way ANOVA with a Tukey multiple comparisons post-test using log-transformed data. The group mean is represented±SD. Each symbol represents a mouse.

FIGS. 23A-23D. In vitro expansion and functional characterization of human dual specific CAR T cells. (FIG. 23A) Experimental setup for FIG. 23B (black dashed boxes) and FIG. 23C (blue dashed boxes). (FIG. 23B) Human anti-CD19 CAR T cells from 3 separate donors were left unloaded or were loaded in vitro at MOI 1.0; 1 hour; 4° C. with reovirus. 10⁶ CAR T or CAR(Reo) T cells were co-cultured with autologous CD14⁺ antigen presenting cells at a ratio of 10 CAR:1 CD14⁺ cell. 2, 5, and 8 days later, 10⁵ additional autologous CD14⁺ APC were added to the cultures. On day 10, CD3⁺ T cells were re-isolated by magnetic bead sorting, and 10⁶ T cells were co-cultured with IFNγ pretreated parental Mel888 cells, Mel888-CD19 cells, or reovirus infected Mel888 (MOI 0.1) or VSV infected Mel888 cells (MOI 0.001) at an E:T ratio of 10:1. 24 hours later, IFNγ secreted into the supernatant was measured by ELISA. P values were calculated using a two way repeated measures ANOVA with a Sidak multiple comparisons test. The group mean is represented±SD. Each symbol represents a donor, and connected samples originate from the same donor. (FIG. 23C) Untransduced activated T cells, or CD19 CAR transduced T cells from 2 separate healthy human donors were left unloaded or loaded in vitro at MOI 1.0 with nothing, VSV-TYRP1, VSV-GFP, or LPS (1 hour; 4° C.). 10⁶ UTD, CAR T, or loaded CAR T cells were co-cultured with autologous CD14⁺ antigen presenting cells at a ratio of 10 CAR:1 CD14⁺ cell. 2, 5, and 8 days later, 10⁵ additional autologous CD14⁺ APC were added to the cultures. On day 10, CD3⁺ T cells were re-isolated by magnetic bead sorting, and 10⁶ T cells were co-cultured with IFNγ pretreated parental Hep3B, Mel888 cells, Raji, or Mel888-CD19 cells at an E:T ratio of 10:1 in ELISPOT wells. 48 hours later wells were developed, and the number of spots counted. (FIG. 23D) NSG mice bearing MEL888-CD19 subcutaneous tumors were treated intravenously with PBS, 10⁷ human anti-CD19 CAR T cells (CAR), 10⁷ human anti-CD19 CAR T cells loaded ex vivo with reovirus (4° C., 1 hour, MOI 10) [CAR(Reo)], or with 10⁷ human CD8⁺ T UTD in vitro activated cells loaded ex vivo with reovirus (4° C., 1 hour, MOI 10) [UTD(Reo)]. Tumor size with time is shown. In the CAR(Reo) treated group, three mice in which complete tumor regression had occurred were euthanized due to the development of GVD toxicity at days 47, 54, or 61.

FIGS. 24A-24C. Combination therapy can be effective against TAA_{CAR} loss tumors. (FIG. 24A and FIG. 24B) Mice bearing brainstem tumors composed entirely of CT2AEGFRvIII cells or 10% CT2A-EGFRvIII plus 90% CT2A cells were treated on day 8 with intravenous PBS, 10⁷ pfu reovirus, 10⁷ anti-EGFRvIII CAR T cells, or with 10⁷ anti-EGFRvIII CAR T cells loaded with reovirus (MOI 1) at 4° C. for 1 hour [CAR(Reo)]. On day 15, mice received a systemic boost with PBS, 10⁷ pfu VSV-GFP, or 10⁷ pfu reovirus. n=7-8 mice/group. A Log Rank Mantel-Cox test was performed, and with a multiple comparison Bonferonni correction, statistical significance was set at p=0.017. (FIG. 24C) Splenocytes were recovered from mice at the time of euthanasia due to tumor size or at the end of the experiment of FIG. 24B. 10⁷ splenocytes cells were co-cultured with CT2A or B16-EGFRvIII cells pre-treated for 24 hours with IFNγ at an E:T ratio of 10:1. 48 hours later, levels of IFNγ were measured by ELISA as shown. P values were determined using a one-way ANOVA with a Tukey multiple comparisons post-test using log-transformed data. The group mean is represented±SD. Each symbol represents a mouse.

FIG. 25. Mice bearing subcutaneous B16EGFRvIII tumors were lymphodepleted on day 3 with 5 Gy total body radiation, and treated on day 4 with 10⁷ EGFRvIII CAR T cells and on day 9 with PBS or with VSV-mIFNβ intravenously (IV) (10⁷ pfu) or intratumorally (IT) (5×10⁷ pfu). On day 16, mice were euthanized, and the skin/receded subcutaneous tumor and spleen were isolated to enumerate CD8⁺ CAR T cells by flow cytometry. In a separate cohort of mice, blood was collected on day 17, and CD8⁺ CAR T cells were quantified by flow cytometry.

FIG. 26 shows sample gating schemes for CD8 CAR T and tetramer staining in splenocytes and subcutaneous tumors.

FIGS. 27A-27C. Expansion of dual specific CAR T in non-lymphodepleted mice. (FIG. 27A) Mice bearing subcutaneous B16EGFRvIII tumors were treated on day 4 with 10⁷ EGFRvIII CAR T cells and on day 9 with PBS or with VSV-mIFNβ intravenously (IV) intratumorally (IT) (5×10⁷ pfu). On day 16, CD8⁺ endogenous and CAR T cells (identified by Thy1.1 expression) with TCR reactivity to the VSV N₅₂₋₅₉ H2-Kb immunodominant epitope were enumerated in the spleen and tumor. n=4 mice/group. (FIG. 27B) Representative flow plots for the CD8⁺ Thy1.1⁺ (CAR) or Thy1.1⁻ (endogenous) gates are shown for individual animals from each treatment. (FIG. 27C) The percent CD8 CAR T tetramer positive populations is represented as the mean±SD. Each symbol represents a mouse.

FIGS. 28A-28B. Expression of KLRG1, CD127, and CD62L on endogenous CD8 populations. Mice bearing subcutaneous B16EGFRvIII tumors were lymphodepleted on day 3 with 5 Gy total body radiation, and treated on day 4 with 10⁷ EGFRvIII CAR T cells and on day 9 with PBS or with VSV-mIFNb intravenously (IV) (10⁷ pfu) or intratumorally (IT) (5×10⁷ pfu). Splenocytes were harvested on day 16. (FIG. 28A) CD8 endogenous (Thy1.1-) expression of KLRG1, CD127, and CD62L is represented as group means±SD. Each symbol represents a mouse. n=4 mice/group. (FIG. 28B) Group means of Boolean gating demonstrating co-expression of markers.

FIG. 29. Human anti-CD19 CAR T cells from 3 separate donors were loaded in vitro at MOI 0.01, 0.1, 1.0, 10 or 100 for 1 hour at 4° C. with reovirus. 10⁶ CAR(Reo) T cells were then co-cultured with either Mel888 or Mel888-CD19 target cells at a ratio of 0.1 CAR:1 target cell in order to minimize target cell killing by CAR T. 72 hours later, reovirus released into the supernatant was collected and titered on Vero cells.

### DETAILED DESCRIPTION

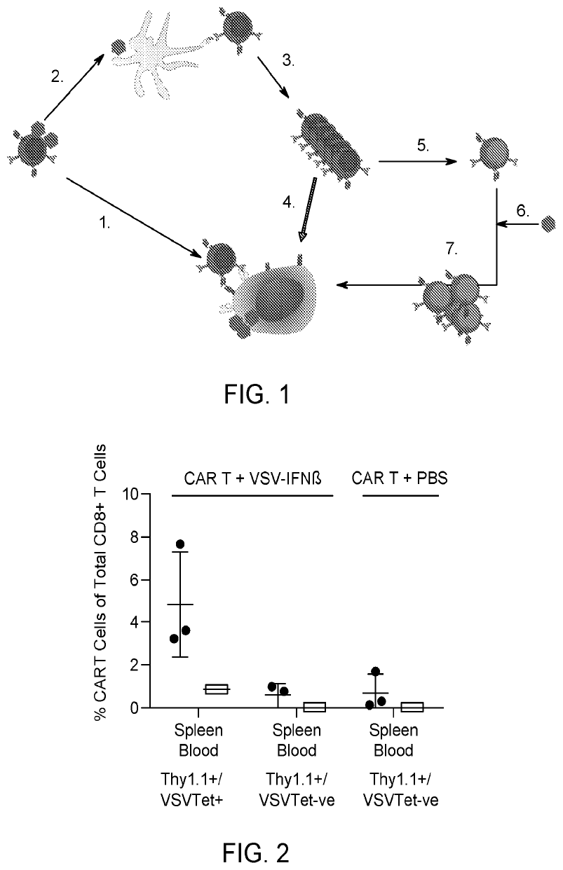

This document provides methods and materials involved in treating cancer. For example, this document provides methods and materials for using T cells (e.g., CAR⁺ T cells) and one or more antigenic compositions (e.g., one or more compositions including one or more antigens) for treating a mammal (e.g., a human) having cancer. In some cases, a mammal (e.g., a human such as a human having cancer) can be administered (a) a population of different T cells engineered to each include a CAR and (b) an antigenic composition (e.g., a composition including one or more viruses such as one or more oncolytic viruses, a composition including one or more viruses designed to express one or more antigens of interest, and/or a composition including one or more antigenic polypeptides of interest) to stimulate in vivo generation of memory T cells specific for one or more of the antigen(s) of the antigenic composition via the endogenous T cell receptors (TCRs), with at least some of those generated memory T cells also expressing the CAR. For example, a mammal can be administered (a) a population of different T cells that each express a particular CAR in addition to their endogenous TCR and (b) an antigenic composition to stimulate in vivo generation of memory T cells from at least a few CAR⁺ T cells of that administered population. It will be appreciated that the mammal's natural T cell population (endogenous T cells) will include some members of the repertoire that also will be stimulated via the antigenic composition, but those cells will only include the endogenous TCR and not the CAR. Those memory T cells generated from the population of CAR⁺ T cells administered to the mammal can have the ability to direct an immune response (e.g., generate a population of CAR⁺ effector T cells) against a target via either the CAR or the endogenous TCR (e.g., an endogenous TCR specific for an antigen of the antigenic composition) of that memory T cell. An exemplary method provided herein can be as shown in FIG. 1.

In some cases, administering (a) a population of different T cells engineered to each include a CAR and (b) an antigenic composition (e.g., a composition including one or more viruses such as one or more oncolytic viruses, a composition including one or more viruses designed to express one or more antigens of interest, and/or a composition including one or more antigenic polypeptides of interest) to mammal can be effective to stimulate a cell-mediated immune response within the mammal. For example, CAR⁺ T cells within the population that are administered to a mammal together with an antigenic composition and that have an endogenous TCR specific for an antigen within that antigenic composition can be stimulated within the mammal via that endogenous TCR and the presence of the antigen. Once stimulated, the CAR⁺ T cells can mediate an immune response against the targets of the CAR. In some cases, administering (a) a population of different T cells engineered to each include a CAR and (b) an antigenic composition (e.g., a composition including one or more viruses such as one or more oncolytic viruses, a composition including one or more viruses designed to express one or more antigens of interest, and/or a composition including one or more antigenic polypeptides of interest) to a mammal can be effective to convert naïve CAR⁺ T cells administered to the mammal into memory T cells within the mammal. In some cases, those generated memory T cells can be dual-specific in that they are CAR⁺ and include an endogenous TCR that recognizes an epitope from the antigenic composition. In such cases, subsequent administration of an antigenic composition that includes that epitope can result in those dual-specific memory T cells expanding quickly and effectively to generate a population of CAR⁺ effector T cells that can hunt and kill cells expressing the target of that CAR.

Any appropriate mammal (e.g., a mammal having cancer) can be treated as described herein. For example, humans, non-human primates (e.g., monkeys), horses, bovine species, porcine species, dogs, cats, mice, and rats can be treated as described herein. In some cases, a human having cancer can be administered (a) a population of different T cells engineered to each include a CAR and (b) an antigenic composition (e.g., a composition including one or more viruses such as one or more oncolytic viruses, a composition including one or more viruses designed to express one or more antigens of interest, and/or a composition including one or more antigenic polypeptides of interest) as described herein. In some cases, a mammal (e.g., a human) treated as described herein can be a pediatric mammal (e.g., human less than 18 years of age). In some cases, a mammal (e.g., a human) treated as described herein can be an adult (e.g., a human that is about 60 years of age or older).

Any appropriate population of T cells can be used as described herein. A population of different T cells engineered to each include a CAR can include any type(s) of T cells. In some cases, a population of T cells can include two or more (e.g., two, three, four, five, or more) different types of T cells. For example, a population of T cells can be a polyclonal population of T cells (e.g., can include a polyclonal population CAR⁺ T cells). In some cases, a population of T cells can be a population of naïve T cells. In some cases, a population of T cells can be a population of stimulated T cells. Examples of T cells that can be designed to express an antigen receptor (e.g., a CAR) and used as described herein include, without limitation, naive T cells (e.g., CD4⁺ naive T cells and/or CD8⁺ naive T cells), cytotoxic T cells (e.g., CD4⁺ CTLs and/or CD8⁺ CTLs), tissue resident memory T cells, and central memory T cells. In some cases, a population of T cells can be obtained from a mammal (e.g., a mammal having cancer). For example, a population of T cells (e.g., a natural T cell population) can be obtained from a mammal to be treated using the materials and methods described herein. In some cases, a population of T cells can be obtained from a donor mammal (e.g., a donor mammal of the same species) as the mammal to be treated using the materials and methods described herein. For example, when treating a human, a population of T cells can be obtained from a donor human. In some cases, when treating a human, a population of T cells can be obtained from a donor transgenic pig donor that was engineered to be compatible with humans. In cases where a donor mammal and the mammal to be treated using the materials and methods described herein are humans, the donor human and the human to be treated using the materials and methods described herein can present the same or similar human leukocyte antigens (HLAs; e.g., can be HLA-matched).

In some cases, a population of T cells described herein (e.g., a population of different T cells engineered to each include a CAR) can include or be representative of an endogenous TCR repertoire. For example, a population of T cells described herein can include greater than about 10³ (e.g., greater than 10⁴, greater than 10⁵, greater than 10⁶, greater than 10⁷, greater than 10⁸, greater than 10⁹, greater than 10¹⁰, or greater than 10¹¹) different TCRs (e.g., different endogenous TCRs). For example, a population of T cells described herein can include from about 10³ to about 10¹¹ (e.g., about 10⁴ to about 10¹¹, about 10⁵ to about 10¹¹, about 10⁶ to about 10¹¹, about 10⁷ to about 10¹¹, about 10⁸ to about 10¹¹, about 10³ to about 10¹⁰, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁵ to about 10⁹, about 10⁶ to about 10⁹, about 10⁷ to about 10⁹, about 10⁸ to about 10⁹, about 10⁵ to about 10⁸, about 10⁶ to about 10⁸, or about 10⁷ to about 10⁸) different TCRs (e.g., different endogenous TCRs).

A T cell (e.g., a CAR⁺ T cell) in a population of T cells described herein (e.g., a population of different T cells engineered to each include a CAR) that can be administered to a mammal (e.g., a human) as described herein can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, the T cells of a population of T cells can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor (e.g., a CAR) expressed in a T cell as described herein include, without limitation, cluster of differentiation 19 (CD19), CD22, CD20, GD2, EGFRvIII, mesothelin, IL-13RA, BCMA, CD138, NKG2-D, HER2/Neu, IL-13RA2, CD137, CD28, B7-H3 (CD276), CD16V, CA-125, MUC-1, epithelial tumor antigen, melanoma-associated antigen, mutated p53, mutated Ras, ERBB2, folate binding protein, HIV-1 envelope glycoprotein gp120, HIV-1 envelope glycoprotein gp41, CD123, CD23, CD30, CD56, c-Met, GD3, HERV-K, IL-11R alpha, kappa chain, lambda chain, CSPG4, and VEGFR2.

When T cells of a population of T cells described herein (e.g., a population of different T cells engineered to each include a CAR) that can be administered to a mammal (e.g., a human) as described herein are CAR⁺ T cells, the CAR can be any appropriate CAR. A CAR can include an antigen-binding domain, an optional hinge, a transmembrane domain, and one or more signaling domains. An antigen-binding domain of a CAR to be used in a CAR⁺ T cell that can be administered to a mammal (e.g., a human) as described herein can be any appropriate antigen-binding domain. In some cases, an antigen-binding domain can include an antibody or a fragment thereof that targets an antigen (e.g., a cancer antigen such as a CD19 polypeptide). Examples of antigen-binding domains include, without limitation, an antigen-binding fragment (Fab), a variable region of an antibody heavy (VH) chain, a variable region of a light (VL) chain, a single chain variable fragment (scFv), and domains from growth factors that bind to a cancer cell-specific receptor (e.g., domains from EGF, PDGR, FGF, TGF, or derivatives thereof). In some cases, an antigen-binding domain can target (e.g., can target and bind to) a tumor-specific antigen. For example, a CAR⁺ T cell described herein can express (e.g., can be engineered to express) a CAR that can bind to a tumor-specific antigen (e.g., an antigen present on cancer cells with minimal, or no, expression on non-cancerous cell types). In some cases, an antigen-binding domain of a CAR can be as described elsewhere (see, e.g., U.S. Patent Application Publication No. 2017/0183418 such as U.S. Patent Application Publication No. 2017/0183418 at paragraph [0015] and the sequence listing; U.S. Patent Application Publication No. 2017/0183413 such as U.S. Patent Application Publication No. 2017/0183413 at paragraph [0049], FIG. 2, Table 9, and the sequence listing; U.S. Patent Application Publication No. 2018/0291079 such as U.S. Patent Application Publication No. 2018/0291079 at paragraphs [0041]-[0045], and Table 4; U.S. Patent Application Publication No. 2020/0289563 such as U.S. Patent Application Publication No. 2020/0289563 at paragraphs [0006]-[0053], [0186]-[0189], and Table 1; and U.S. Patent Application Publication No. 2003/0211097 such as U.S. Patent Application Publication No. 2003/0211097 at paragraphs [0081] and [0211-0215] and the sequence listing.

In some cases, a CAR to be used in a CAR⁺ T cell of a population of T cells described herein (e.g., a population of different T cells engineered to each include a CAR) can include an optional hinge region. In some cases, a hinge region can be located between an antigen-binding domain and a transmembrane domain of a CAR. In some cases, a hinge region can provide a CAR with increased flexibility for the antigen-binding domain. For example, a hinge region can reduce spatial limitations of an antigen-binding domain of a CAR and its target antigen (e.g., to increase binding between an antigen-binding domain of a CAR and its target antigen). Examples of hinge regions that can be used as described herein include, without limitation, a membrane-proximal region from an IgQ a membrane-proximal region from CD8, and a membrane-proximal region from CD28. In some cases, a hinge region of a CAR can be as described elsewhere (see, e.g., U.S. Patent Application Publication No. 2018/0000914 such as U.S. Patent Application Publication No. 2018/0000914 at paragraph [0168], and Table 1; U.S. Patent Application Publication No. 2017/0183418 such as U.S. Patent Application Publication No. 2017/0183418 at paragraphs [0034], [0037], [0040], and Table 2; U.S. Patent Application Publication No. 2017/0183413 such as U.S. Patent Application Publication No. 2017/0183413 at paragraph [0116]; and U.S. Patent Application Publication No. 2017/0145094 such as U.S. Patent Application Publication No. 2017/0145094 at paragraph [0104].

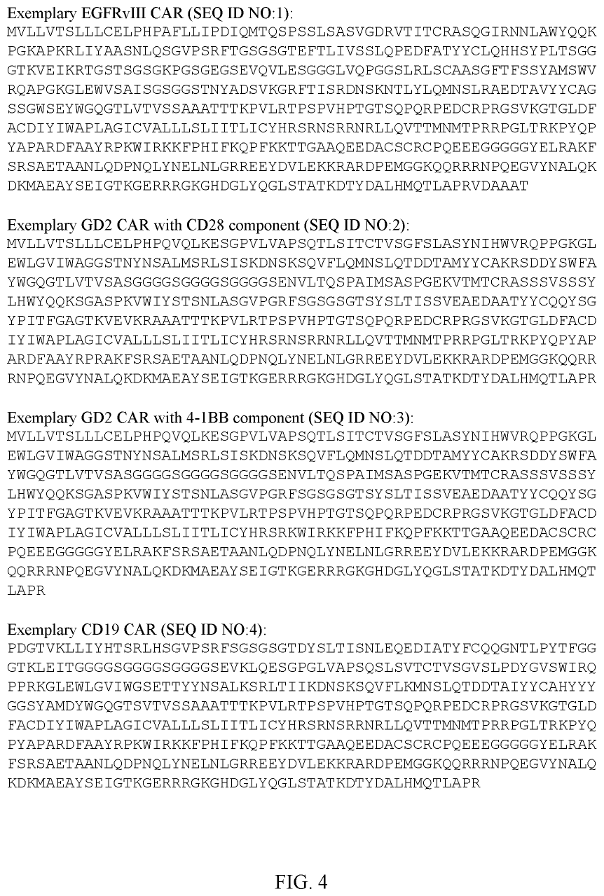

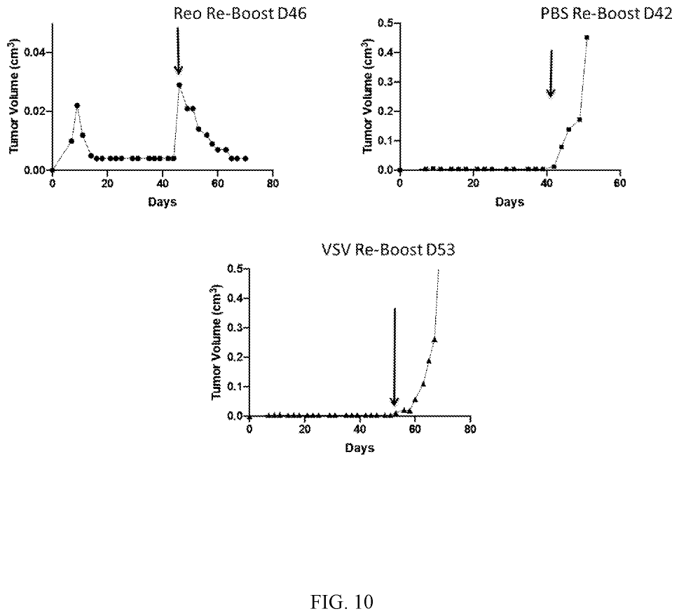

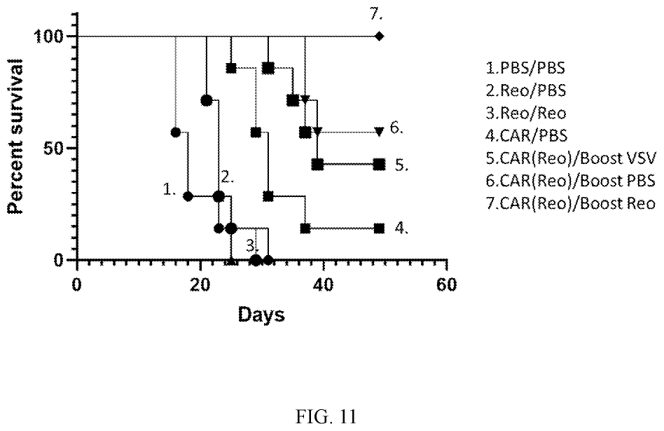

A transmembrane domain of a CAR to be used in a CAR⁺ T cell of a population of T cells described herein (e.g., a population of different T cells engineered to each include a CAR) that can be administered to a mammal (e.g., a human) as described herein can include any appropriate transmembrane domain. A transmembrane domain can be located between an antigen-binding domain and a signaling domain of a CAR and/or located between a hinge and a signaling domain of a CAR. In some cases, a transmembrane domain can provide structural stability for the CAR. For example, a transmembrane domain can include a structure (e.g., a hydrophobic alpha helix structure) that can span a cell membrane and can anchor the CAR to the plasma membrane. Examples of transmembrane domains that can be used as described herein include, without limitation, CD3ζ transmembrane domains, CD4 transmembrane domains, CD8 (e.g., a CD8a) transmembrane domains, CD28 transmembrane domains, CD16 transmembrane domains, and erythropoietin receptor transmembrane domains. In some cases, a transmembrane domain of a CAR can be as described elsewhere (see, e.g., U.S. Patent Application Publication No. 2016/0120906 such as U.S. Patent Application Publication No. 2016/0120906 at paragraphs [0155], [0161], [0269], FIG. 4, and FIG. 11; U.S. Patent Application Publication No. 2019/0209616 such as U.S. Patent Application Publication No. 2019/0209616 at paragraph [0026]; U.S. Patent Application Publication No. 2018/0000914 such as U.S. Patent Application Publication No. 2018/0000914 at paragraphs [0168]-[0171]; U.S. Patent Application Publication No. 2017/0183418 such as U.S. Patent Application Publication No. 2017/0183418 at paragraphs [0116]-[0118]; U.S. Patent Application Publication No. 2017/0183413 such as U.S. Patent Application Publication No. 2017/0183413 at paragraphs [0116]-[0118]; and U.S. Patent Application Publication No. 2017/0145094 such as U.S. Patent Application Publication No. 2017/0145094 at paragraphs [0104]-[0107].

The signaling domain(s) of a CAR to be used in a CAR⁺ T cell of a population of T cells described herein (e.g., a population of different T cells engineered to each include a CAR) that can be administered to a mammal (e.g., a human) as described herein can include any appropriate signaling domain or combination of signaling domains (e.g., a combination of two, three, or four signaling domains). In some cases, a signaling domain of a CAR can be an intracellular signaling domain normally found within T cells or NK cells. Examples of signaling domains that can be used as described herein include, without limitation, CD2 signaling domains, CD3ζ signaling domains, CD28 signaling domains, Toll-like receptor (TLR) signaling domains (e.g., TLR3 or TLR4 signaling domains), CD27 intracellular signaling domains, OX40 (CD134) intracellular signaling domains, 4-1BB (CD137) intracellular signaling domains, CD278 intracellular signaling domains, DAP10 intracellular signaling domains, DAP12 intracellular signaling domains, FceRly intracellular signaling domains, CD278 intracellular signaling domains, CD122 intracellular signaling domains, CD132 intracellular signaling domains, CD70 intracellular signaling domains, cytokine receptor intracellular signaling domains, and CD40 intracellular signaling domains. In some cases, a CAR for use as described herein can be designed to be a first generation CAR having a CD3ζ intracellular signaling domain. In some cases, a CAR for use as described herein can be designed to be a second generation CAR having a CD28 intracellular signaling domain followed by a CD3ζ intracellular signaling domain. In some cases, a CAR for use as described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, the intracellular signaling domain(s) of a CAR can be as described elsewhere (see, e.g., U.S. Patent Application Publication No. 2018/0000914 such as U.S. Patent Application Publication No. 2018/0000914 at paragraphs [0164]-[0167]; and U.S. Patent Application Publication No. 2017/0183413 such as U.S. Patent Application Publication No. 2017/0183413 at paragraphs [0112]-[0115].

Examples of CARs that can be expressed on one or more T cells of a population of T cells described herein (e.g., a population of different T cells engineered to each include a CAR) include, without limitation, EGFRvIII CARs, GD2 CARs, IL-13RA CARs, CD19 CARs, BCMA CARs, CD138 CARs, NKG2-D CARs, HER2 CARs, CD137 CARs, and B7-H3 CARs. Exemplary amino acid sequences for such CARs are set forth in FIG. 4.

Any appropriate method can be used to express an antigen receptor (e.g., a CAR) on the surface of a T cell of a population of T cells described herein (e.g., a population of different T cells engineered to each include a CAR) that can be administered to a mammal (e.g., a human) as described herein. For example, a nucleic acid encoding an antigen receptor (e.g., a CAR) can be introduced into one or more T cells of a population of T cells to be administered to a mammal as described herein. In some cases, viral transduction can be used to introduce a nucleic acid encoding an antigen receptor (e.g., a CAR) into a non-dividing a cell. A nucleic acid encoding an antigen receptor (e.g., a CAR) can be introduced in a T cell using any appropriate method. In some cases, a nucleic acid encoding an antigen receptor (e.g., a CAR) can be introduced into a T cell by transduction (e.g., viral transduction using a retroviral vector such as a lentiviral vector) or transfection. In some cases, a nucleic acid encoding an antigen receptor (e.g., a CAR) can be introduced ex vivo into one or more T cells. For example, ex vivo engineering of T cells expressing an antigen receptor (e.g., a CAR) can include transducing isolated T cells with a lentiviral vector encoding an antigen receptor (e.g., a CAR). In cases where T cells are engineered ex vivo to express an antigen receptor (e.g., a CAR), the T cells can be obtained from any appropriate source (e.g., a mammal such as the mammal to be treated or a donor mammal).

Any number of T cells of a population of T cells described herein (e.g., a population of different T cells engineered to each include a CAR) can be engineered to include a CAR (e.g., can be CAR⁺ T cells). In some cases, at least half of the T cells within a population of T cells to be administered to a mammal as described herein can include a CAR (e.g., can be CAR⁺ T cells). In some cases, from about 25 percent to about 100 percent (e.g., from about 25 percent to about 99 percent, from about 25 percent to about 95 percent, from about 25 percent to about 90 percent, from about 50 percent to about 100 percent, from about 50 percent to about 99 percent, from about 50 percent to about 95 percent, from about 50 percent to about 90 percent, about 50 percent to about 85 percent, about 50 percent to about 80 percent, or from about 25 percent to about 75 percent) of the T cells within a population of T cells to be administered to a mammal as described herein can be CAR⁺ T cells. In some cases, 75 percent or less (e.g., 75 percent or less, 70 percent or less, 60 percent or less, 50 percent or less, 40 percent or less, 30 percent or less, 25 percent or less, 20 percent or less, 15 percent or less, 10 percent or less, 5 percent or less, or 2.5 percent or less) of the T cells within a population of T cells to be administered to a mammal as described herein can lack a CAR (e.g., can be CAR⁻ T cells).

In some cases, the CAR⁺ T cells of a population of T cells to be administered to a mammal as described herein (e.g., a population of different T cells engineered to each include a CAR) can be engineered to each include the same CAR.

In some cases, the CAR⁺ T cells in a population of T cells to be administered to a mammal as described herein (e.g., a population of different T cells engineered to each include a CAR) can be engineered so that some of those CAR⁺ T cells of the population express one CAR and others express a different CAR. In some cases, the CAR⁺ T cells in a population of T cells to be administered to a mammal as described herein (e.g., a population of different T cells engineered to each include a CAR) can be engineered so that the population includes T cells expressing a first CAR, T cells expressing a second CAR that is different from the first CAR, and T cells expressing a third CAR that is different from the first and second CARs. In some cases, the CAR⁺ T cells in a population of T cells to be administered to a mammal as described herein (e.g., a population of different T cells engineered to each include a CAR) can be engineered so that the population includes T cells expressing a first CAR, T cells expressing a second CAR that is different from the first CAR, T cells expressing a third CAR that is different from the first and second CARs, and T cells expressing a fourth CAR that is different from the first, second, and third CARs.

In some cases, an individual T cell in a population of T cells described herein (e.g., a population of different T cells engineered to each include a CAR) can be engineered to include two or more (e.g., two, three, four, five, or more) different CARs.

An antigenic composition (e.g., a composition including one or more viruses such as one or more oncolytic viruses, a composition including one or more viruses designed to express one or more antigens of interest, and/or a composition including one or more antigenic polypeptides of interest) can include any type of antigen(s). For example, an antigen that can be used in an antigenic composition described herein can be a virus. In some cases, a virus that can be used in an antigenic composition described herein can be an oncolytic virus. In some cases, a virus that can be used in an antigenic composition described herein can be an immunogenic virus. In some cases, a virus that can be used in an antigenic composition described herein can be replication competent. In some cases, a virus that can be used in an antigenic composition described herein can be non-pathogenic (e.g., non-pathogenic to a mammal being treated as described herein). For example, a virus that can be used in an antigenic composition described herein can be genetically modified to render it non-pathogenic to a mammal to be treated. In some cases, a virus that can be used in an antigenic composition described herein can infect dividing cells (e.g., can infect only dividing cells). In some cases, a virus that can be used in an antigenic composition described herein can infect non-dividing cells (e.g., can infect only non-dividing cells). In some cases, a virus that can be used in an antigenic composition described herein can infect a cancer cell expressing an antigen targeted by a CAR⁺ T cell administered together with the antigen (e.g., an antigenic composition containing the virus). In some cases, a virus that can be used in an antigenic composition described herein can bud through the endoplasmic reticulum. In some cases, a virus that can be used in an antigenic composition described herein can bind to a cellular receptor (e.g., bind to a cellular receptor to facilitate viral entry into a cell). Examples of viruses that can be used in an antigenic composition described herein include, without limitation, Rhabdoviruses (e.g., vesiculoviruses (VSVs), and Maraba viruses), reoviruses, adenoviruses, vaccinia viruses, Newcastle disease viruses, polioviruses, HSV viruses, and measles viruses.

When an antigen used in an antigenic composition described herein is a virus (e.g., an oncolytic virus), the virus can express (e.g., can be designed to express) one or more antigens (e.g., one or more antigens heterologous to that virus). In some cases, an antigen expressed by a virus (e.g., a heterologous antigen) that can be used in an antigenic composition described herein can be a polypeptide. In some cases, an antigen expressed by a virus (e.g., a heterologous antigen) that can be used in an antigenic composition described herein is not endogenous to the mammal being treated as described herein. In some cases, an antigen expressed by a virus (e.g., a heterologous antigen) can be a full-length antigenic polypeptide. In some cases, an antigen expressed by a virus (e.g., a heterologous antigen) can be a fragment of a full-length polypeptide (e.g., provided that the fragment retains an antigenic property within a mammal being treated). In some cases, an antigen expressed by a virus (e.g., a heterologous antigen) can be derived from a full-length polypeptide (e.g., provided that the fragment retains an antigenic property within a mammal being treated). Examples of antigens that can be expressed by a virus (e.g., a heterologous antigen) in an antigenic composition described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-CoV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof.

A virus expressing one or more antigens can be generated using any appropriate method. In some cases, nucleic acid encoding an antigen (e.g., a heterologous antigen) can be introduced into the genome of a virus such that the antigen is expressed. Nucleic acid encoding an antigen (e.g., a heterologous antigen) can be introduced in the genome of a virus using any appropriate method. In some cases, nucleic acid encoding an antigen (e.g., a heterologous antigen) can be introduced into the genome of a virus by homologous recombination techniques, molecular cloning, and gene editing techniques (e.g., the CRISPR-Cas9 System).

In some cases, an antigen that can be used in an antigenic composition can be an antigenic polypeptide. For example, an antigenic polypeptide that can be used in an antigenic composition described herein can be a polypeptide that is not endogenous to the mammal being treated as described herein. In some cases, an antigenic polypeptide used as described herein can be a full-length antigenic polypeptide. In some cases, an antigenic polypeptide used as described herein can be a fragment of a full-length polypeptide (e.g., provided that the fragment retains an antigenic property within the mammal being treated). In some cases, an antigenic polypeptide used as described herein can be derived from a full-length polypeptide (e.g., provided that the fragment retains an antigenic property within the mammal being treated). In some cases, an antigenic polypeptide can be foreign (e.g., exogenous) to a mammal (e.g., a human) to be treated as described herein. In some cases, an antigenic polypeptide used as described herein can be a polypeptide that has no natural counterparts in the mammal (e.g., the human) to be treated as described herein. In some cases, an antigenic polypeptide used as described herein can be a synthetic polypeptide (e.g., a synthetic polypeptide designed to be a potent immunogenic polypeptide). In some cases, an antigenic polypeptide used as described herein can have no natural counterparts in nature. Examples of antigenic polypeptides that can be included in an antigenic composition to be administered to a mammal (e.g., a human) as described herein include, without limitation, ovalbumin polypeptides (OVA) and antigenic fragments thereof, TYRP1 polypeptides and antigenic fragments thereof, TYRP2 polypeptides and antigenic fragments thereof, tyrosinase polypeptides and antigenic fragments thereof, CEA polypeptides and antigenic fragments thereof, MART1 polypeptides and antigenic fragments thereof, MART2 polypeptides and antigenic fragments thereof, SARS-CoV-2 spike polypeptides and antigenic fragments thereof, VSV-G polypeptides and antigenic fragments thereof, reovirus surface polypeptides and antigenic fragments thereof, adenovirus coat polypeptides and antigenic fragments thereof, CSDE1 polypeptides and antigenic fragments thereof, and superantigen polypeptides (e.g., Streptococcal pyrogenic exotoxins (SPE), Staphylococcal enterotoxins (SE), and enterotoxogenic E. coli (ETEC) enterotoxins) and antigenic fragments thereof. In some cases, an antigenic composition described herein can contain one or more antigens of interest in the absence of any virus particles.

In some cases, an antigenic composition described herein (e.g., a composition including one or more viruses such as one or more oncolytic viruses, a composition including one or more viruses designed to express one or more antigens of interest, and/or a composition including one or more antigenic polypeptides of interest) can contain one or more antigens other than polypeptides. Examples of antigens other than polypeptides that can be used in a composition described herein include, without limitation, polysaccharides (e.g., type 3 S. pneumoniae polysaccharide (Pn3P) and/or polysaccharides of MUC-1) and lipids.

In some cases, a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal at the same time (e.g., in a single composition). In some cases when a population of T cells described herein and an antigenic composition described herein are formulated as a single composition, the T cells can be loaded with the antigenic compositions. For example, T cells in a population of T cells described herein can be contacted with an antigenic composition (e.g., an antigenic composition containing viruses such as oncolytic viruses) such that the antigen(s) (e.g., the viruses) bind to the T cells. In some cases, antigens (e.g., the viruses such as oncolytic viruses) that are loaded onto T cells (e.g., CAR⁺ T cells) can be covalently bound to the surface of the T cells. In some cases, antigens (e.g., the viruses such as oncolytic viruses) that are loaded onto the surface of T cells (e.g., CAR⁺ T cells) can be non-covalently bound to the T cells. In some cases, antigens (e.g., viruses such as oncolytic viruses) that are loaded onto the surface of T cells (e.g., CAR⁺ T cells) can be bound to the T cells through envelope receptor interactions, electrostatic interactions, and/or non-specific interactions between the virus and the T cell surface glycocalyx.

In some cases when a population of T cells described herein (e.g., a population of different T cells engineered to each include a CAR) and an antigenic composition including one or more viruses (e.g., one or more oncolytic viruses) and/or one or more viruses designed to express one or more antigens of interest, are formulated as a single composition, at least some of the T cells can be infected with the virus(es). For example, T cells in a population of T cells described herein can be contacted with antigenic composition including one or more viruses (e.g., one or more oncolytic viruses) and/or one or more viruses designed to express one or more antigens of interest such that the virus(es) can infect at least some of the T cells within the population of T cells.

In some cases when a population of T cells described herein (e.g., a population of different T cells engineered to each include a CAR) and an antigenic composition including one or more viruses (e.g., one or more oncolytic viruses) and/or one or more viruses designed to express one or more antigens of interest, are formulated as a single composition, the population of T cells and the composition containing the viruses can be combined into that single composition in a manner that results in minimal viral infection of the T cells. For example, a population of T cells described herein (e.g., a population of different T cells engineered to each include a CAR) and an antigenic composition including one or more viruses (e.g., one or more oncolytic viruses) and/or one or more viruses designed to express one or more antigens of interest can be combined and incubated at a temperature of about 2° C. to about 8° C. (e.g., about 2° C. to about 6° C., about 2° C. to about 5° C., about 3° C. to about 8° C., about 4° C. to about 8° C., about 3° C. to about 6° C., about 3° C. to about 5° C., or about 4° C.) for 3 hours or less (e.g., 2.5 hours or less, 2 hours or less, 1.5 hours or less, 1 hour or less, or about 1 hour) prior to being administered to the mammal or prior to being frozen for administration to the mammal at a later time. In such cases, the viruses can infect less than about 10 percent (e.g., less than about 9 percent, less than about 8 percent, less than about 7 percent, less than about 7 percent, or less than about 5 percent) of the T cells of the population. For example, when T cells in a population of T cells described herein are loaded with an antigenic composition including one or more viruses, the viruses can infect less than about 5 percent of the T cells of that population.

In some cases, a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can include an antigenic composition including one or more antigenic polypeptides of interest in the absence of viruses. For example, a composition including a population of T cells described herein and an antigenic composition including one or more antigenic polypeptides of interest can lack the presence of virus particles.

When a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as a single composition, the composition including a population of different T cells engineered to each include a CAR described herein and an antigenic composition described herein can be administered to a mammal by any appropriate route. For example, a composition including (a) a population of T cells described herein and (b) an antigenic composition described herein can be administered locally or systemically. In some cases, a composition including (a) a population of different T cells engineered to each include a CAR described herein and (b) an antigenic composition described herein can be designed for parenteral (e.g., subcutaneous, intramuscular, intravenous, intraperitoneal, and intradermal) administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

In some cases, a composition including (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered systemically by intravenous injection to a mammal (e.g., a human).

In some cases, a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered a mammal (e.g., a human) separately. For example, a composition including a population of different T cells engineered to each include a CAR and an antigenic composition can be administered to a mammal at the same time (e.g., concurrently) as independent compositions. When a composition including a population of different T cells engineered to each include a CAR and an antigenic composition are administered concurrently, the composition including a population of different T cells engineered to each include a CAR and the antigenic composition can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered a mammal (e.g., a human) at different times. When a composition including a population of different T cells engineered to each include a CAR and an antigenic composition are administered at different times, the composition including a population of different T cells engineered to each include a CAR and the antigenic composition can be administered to a mammal with from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) between each administration.

When a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as separate compositions (e.g., administered concurrently as separate compositions or administered as separate compositions with from about 16 minutes to about 48 hours between each administration), each composition can be administered to a mammal by any appropriate route. In some cases, a composition including a population of T cells described herein and an antigenic composition described herein can be administered by the same route. In some cases, a composition including a population of T cells described herein and an antigenic composition described herein can be administered by different routes.

A composition including a population of T cells described herein (e.g., a population of different T cells engineered to each include a CAR) can be administered to a mammal by any appropriate route. For example, a composition including a population of T cells described herein can be administered locally or systemically. In some cases, a composition including a population of different T cells engineered to each include a CAR described herein can be designed for parenteral (e.g., subcutaneous, intramuscular, intravenous, intraperitoneal, and intradermal) administration. In some cases, a composition including a population of T cells described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

An antigenic composition described herein (e.g., a composition including one or more viruses such as one or more oncolytic viruses, a composition including one or more viruses designed to express one or more antigens of interest, and/or a composition including one or more antigenic polypeptides of interest) can be administered to a mammal by any appropriate route. For example, an antigenic composition described herein can be administered locally or systemically. In some cases, an antigenic composition described herein can be designed for oral or parenteral (e.g., subcutaneous, intramuscular, intravenous, intraperitoneal, and intradermal) administration. In some cases, an antigenic composition described herein can be administered via an intra-tumoral administration. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intra-tumoral administration to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intra-tumoral administration to the mammal.

In some cases, a composition including a population of T cells described herein (e.g., a population of different T cells engineered to each include a CAR) can be administered by intraperitoneal injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intraperitoneal injection to the mammal.

In some cases, a composition including a population of T cells described herein (e.g., a population of different T cells engineered to each include a CAR) can be administered by subcutaneous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by subcutaneous injection to the mammal.

In some cases, a composition including a population of T cells described herein (e.g., a population of different T cells engineered to each include a CAR) can be administered by intramuscular injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intramuscular injection to the mammal.

In some cases, a composition including a population of T cells described herein (e.g., a population of different T cells engineered to each include a CAR) can be administered by intravenous injection to a mammal (e.g., a human), and an antigenic composition described herein (e.g., a composition including one or more viruses such as one or more oncolytic viruses, a composition including one or more viruses designed to express one or more antigens of interest, and/or a composition including one or more antigenic polypeptides of interest) can be administered orally to the mammal.

When a composition including a population of T cells described herein (e.g., a population of different T cells engineered to each include a CAR) and an antigenic composition described herein (e.g., a composition including one or more viruses such as one or more oncolytic viruses, a composition including one or more viruses designed to express one or more antigens of interest, and/or a composition including one or more antigenic polypeptides of interest) are administered as separate compositions (e.g., administered concurrently as separate compositions or administered as separate compositions with from about 0 seconds to about 15 minutes between each administration), the population of T cells can be administered first, and the antigenic composition administered second, or vice versa.

In some cases, administering (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest) to mammal (e.g., a human) can be effective to generate memory T cells in vivo. For example, one or more T cells in a population of T cells described herein that are administered to a mammal can be converted into a memory T cell (e.g., a dual-specific memory T cell that is CAR⁺ and that has an endogenous TCR specific for an antigen present within the antigenic composition administered to the mammal). Examples of types of memory T cells that can be generated from T cells (e.g., CAR⁺ T cells) administered to a mammal as described herein include, without limitation, central memory T cells (T_{CM} cells), effector memory T cells (T_{EM} cells), terminally differentiated effector memory T cells (T_{EMRA} cells), and tissue resident memory T cells (T_{RM}). Memory T cells generated within a mammal by the administration of a population of T cells described herein and an antigenic composition described herein to the mammal can be dual-specific. For example, memory T cells generated within a mammal by the administration of CAR⁺ T cells and an antigenic composition to the mammal can target an antigen recognized by the CAR via the CAR and an antigen present in the antigenic composition via an endogenous TCR.

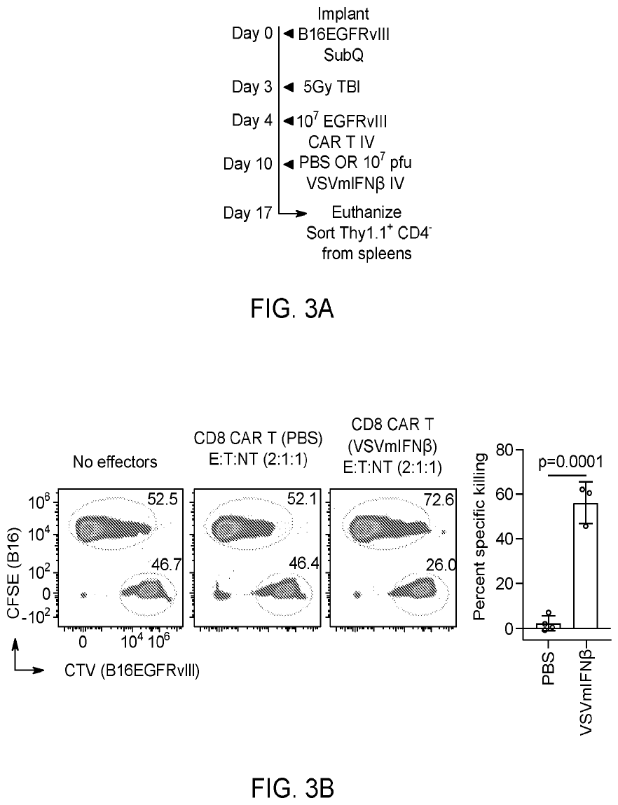

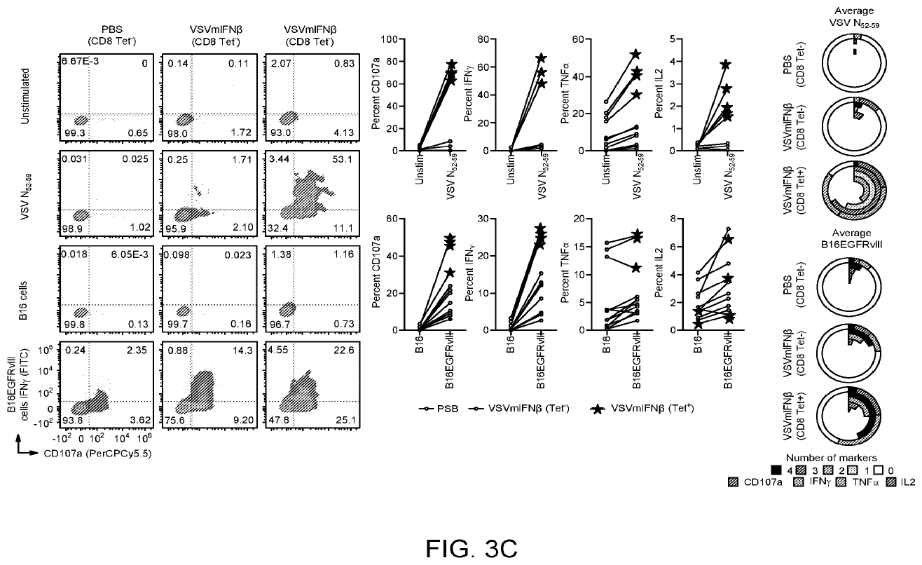

In some cases, memory T cells generated within a mammal (e.g., a human) as described herein (e.g., by administering a population of different T cells expressing one or more CARs and one or more antigenic compositions) can be more functional against cancer cells present in the mammal (e.g., as compared to T cells such as CAR⁺ T cells that are administered without an antigenic composition and/or without subsequently administering (e.g., boosting) with an antigenic composition). In some cases, memory T cells generated within a mammal (e.g., a human) as described herein (e.g., by administering a population of different T cells expressing one or more CARs and one or more antigenic compositions) can be more functional against cancer cells present in the mammal (e.g., as compared to T cells such as CAR⁺ T cells that are administered without an antigenic composition and/or without subsequently administering (e.g., boosting) with an antigenic composition) as assessed by, for example, increased cytotoxicity against CAR target cancer cells and/or increased IFN-7 secretion upon stimulation with cancer cells. See, e.g., FIGS. 3A-3C.

In some cases, memory T cells generated within a mammal (e.g., a human) as described herein (e.g., by administering a population of different T cells expressing one or more CARs and one or more antigenic compositions) can persist longer within the mammal (e.g., as compared to T cells such as CAR T cells that are administered without an antigenic composition and/or without subsequently administering (e.g., boosting) with an antigenic composition). For example, the materials and methods described herein can be used to generate T cells that can persist within a mammal (e.g., a human) for from about 40 days to about 2 years (e.g., from about 40 days to about 1.5 years, from about 40 days to about 1 year, from about 40 days to about 11 months, from about 40 days to about 10 months, from about 40 days to about 9 months, from about 40 days to about 8 months, from about 40 days to about 7 months, from about 40 days to about 6 months, from about 50 days to about 200 days, from about 50 days to about 180 days, from about 50 days to about 160 days, from about 50 days to about 150 days, from about 50 days to about 125 days, or from about 80 days to about 1 year). See, e.g., FIG. 2.

Once memory T cells (e.g., T_{RM} CAR⁺ T cells) are generated within a mammal, the mammal can be administered a second antigenic composition (e.g., a composition including one or more viruses such as one or more oncolytic viruses, a composition including one or more viruses designed to express one or more antigens of interest, and/or a composition including one or more antigenic polypeptides of interest) one or more (e.g., one, two, three, four, five, or more times). For example, a mammal can be subsequently administered (e.g., can be boosted with) a second antigenic composition from about 5 days to about 5 years (e.g., from about 5 days to about 5 years, from about 7 days to about 5 years, from about 10 days to about 5 years, from about 14 days to about 5 years, from about 21 days to about 5 years, from about 1 month to about 5 years, from about 2 months to about 5 years, from about 3 months to about 5 years, from about 4 months to about 5 years, from about 5 months to about 5 years, from about 6 months to about 5 years, from about 5 days to about 4.5 years, from about 5 days to about 4 years, from about 5 days to about 3.5 years, from about 5 days to about 3 years, from about 5 days to about 2.5 years, from about 5 days to about 2 years, from about 5 days to about 1.5 years, from about 5 days to about 1 year, from about 5 days to about 10 months, from about 5 days to about 8 months, from about 5 days to about 6 months, from about 5 days to about 4 months, from about 5 days to about 3 months, from about 5 days to about 2 months, from about 5 days to about 1 month, from about 5 days to about 20 days, from about 5 days to about 15 days, from about 5 days to about 10 days, from about 10 days to about 200 days, from about 20 days to about 200 days, from about 30 days to about 200 days, from about 40 days to about 200 days, from about 50 days to about 200 days, from about 10 days to about 175 days, from about 10 days to about 150 days, from about 10 days to about 125 days, from about 10 days to about 100 days, from about 50 days to about 110 days, or from about 60 days to about 100 days) after having been administered (a) a population of different T cells engineered to each include a CAR and (b) a first antigenic composition (e.g., a composition including one or more viruses such as one or more oncolytic viruses, a composition including one or more viruses designed to express one or more antigens of interest, and/or a composition including one or more antigenic polypeptides of interest). In some cases, a mammal can be administered a second antigenic composition (e.g., a boost) from about 5 days to about 150 days (e.g., from about 60 days to about 100 days) after having been administered (a) a population of different T cells engineered to each include a CAR and (b) a first antigenic composition. For example, a mammal can be administered a second antigenic composition from about 5 days to about 8 days (e.g., about 7 days) after having been administered a population of different T cells engineered to each include a CAR and a first antigenic composition.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include the same antigen(s) as a first antigenic composition that was administered together with a population of T cells described herein (e.g., a population of different T cells engineered to each include a CAR).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more different antigens as compared to the first antigenic composition that was administered together with a population of T cells described herein (e.g., a population of different T cells engineered to each include a CAR).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can lack T cells (e.g., can lack a population of T cells engineered to each include a CAR). For example, a mammal (e.g., a human) can be administered (a) a population of T cells as described herein (e.g., a population of different T cells engineered to each include a CAR) and (b) a first antigenic composition as described herein (e.g., a composition including one or more viruses such as one or more oncolytic viruses, a composition including one or more viruses designed to express one or more antigens of interest, and/or a composition including one or more antigenic polypeptides of interest). Then, after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of that population of T cells and that first antigenic composition, the mammal (e.g., the human) can be administered a second antigenic composition that does not include T cells. In some cases, that second antigenic composition can be identical to the first antigenic composition administered to the mammal. For example, in some cases, the first antigenic composition administered to the mammal can include one or more oncolytic viruses (e.g., one or more VSV viruses, one or more reoviruses, one or more measles viruses, or combinations thereof), and the second antigenic composition administered to the mammal can include those same one or more oncolytic viruses. In some cases, that second antigenic composition can be different from the first antigenic composition administered to the mammal. For example, in some cases, the first antigenic composition administered to the mammal can include one or more viruses designed to express one or more antigens of interest, and the second antigenic composition administered to the mammal can include one or more of those antigens of interest that were expressed by the viruses of the first antigenic composition with that second antigenic composition lacking the viruses. In some cases, a mammal (e.g., a human) can be treated as described herein with the initially administered population of T cells (e.g., a population of different T cells engineered to each include a CAR) being the only T cells that are administered to the mammal.

In some cases, a mammal (e.g., a human) can be treated as described herein with the initially administered population of T cells (e.g., a population of different T cells engineered to each include a CAR) and first antigenic composition can be subsequently treated with multiple rounds of additional populations of T cells (e.g., an additional population of different T cells engineered to each include a CAR) and/or additional antigenic compositions.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more viruses (e.g., one or more oncolytic viruses).

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can include one or more antigenic polypeptides of interest. For example, a second antigenic composition can include one or more antigenic polypeptides of interest that were expressed by a virus present in a first antigen composition. For example, a second antigenic composition can include one or more antigenic polypeptides of interest that were expressed by a virus present in a first antigen composition, and can lack virus particles.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) as the sole active agent to stimulate the memory T cells generated within the mammal.

In some cases, a second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal (e.g., a human) together with one or more additional agents that can stimulate memory T cells within the mammal (e.g., can stimulate the memory T cells generated within the mammal as described herein). Examples of additional agents (e.g., other than a second antigen composition) that can be used to stimulate memory T cells within a mammal include, without limitation, pathogens and TLR agonists.

A second antigenic composition (e.g., a composition including one or more viruses such as one or more oncolytic viruses, a composition including one or more viruses designed to express one or more antigens of interest, and/or a composition including one or more antigenic polypeptides of interest) can be administered to a mammal by any appropriate route. For example, a second antigenic composition described herein can be administered locally or systemically. In some cases, a second antigenic composition described herein can be designed for oral or parenteral (e.g., subcutaneous, intramuscular, intravenous, intraperitoneal, and intradermal) administration. When being administered orally, a composition can be in the form of a pill, tablet, or capsule. Compositions suitable for parenteral administration include aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents. The composition can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a second antigenic composition described herein (e.g., a composition including one or more viruses such as one or more oncolytic viruses, a composition including one or more viruses designed to express one or more antigens of interest, and/or a composition including one or more antigenic polypeptides of interest) can be administered by intravenous injection to the mammal.

In some cases, administration of a second antigenic composition (e.g., a composition including one or more viruses such as one or more oncolytic viruses, a composition including one or more viruses designed to express one or more antigens of interest, and/or a composition including one or more antigenic polypeptides of interest) as described herein (e.g., a boost) can be effective to activate memory T cells (e.g., dual-specific memory T cells) generated as described herein. For example, a subsequent administration (e.g., a boost) of a second antigenic composition can be used to rapidly reactivate memory T cells generated by administering a population of T cells described herein and a first antigenic composition described herein to generate effector T cells that are dual-specific (e.g., effector T cells that are CAR⁺ and positive for an endogenous TCR that recognizes an antigen that was present in both the first antigenic composition and the boost).

In some cases, the materials and methods provided herein can be used to treat a mammal (e.g., a human) having cancer. For example, a mammal in need thereof (e.g., a mammal having cancer) can be administered (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest) to reduce or eliminate the number of cancer cells present within the mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

In some cases, the materials and methods provided herein can be used to improve survival of a mammal (e.g., a human) having cancer. For example, a mammal in need thereof (e.g., a mammal having cancer) can be administered (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest) to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, the methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can be administered or instructed to self-administer (a) a population of T cells described herein (e.g., a population of different T cells engineered to each include a CAR) and (b) an antigenic composition described herein (e.g., a composition including one or more viruses such as one or more oncolytic viruses, a composition including one or more viruses designed to express one or more antigens of interest, and/or a composition including one or more antigenic polypeptides of interest). Then, in some cases, after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months) after the latter administration of the population of T cells and the antigenic composition, the mammal can be administered or instructed to self-administer a second antigenic composition that includes at least some of the antigens present in the first antigenic composition administered to the mammal.

The materials and methods described herein can be used to treat a mammal (e.g., a human) having any type of cancer. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can express a tumor-specific antigen (e.g., an antigenic substance produced by a cancer cell). Examples of cancers that can be treated as described herein include, without limitation, brain cancers (e.g., brain stem gliomas such as high-grade gliomas (HGGs)), pancreatic cancers (e.g., pancreatic adenocarcinoma), bile duct cancers (e.g., cholangiocarcinoma), lung cancers (e.g., mesothelioma), skin cancers (e.g., melanoma), prostate cancers, breast cancers, ovarian cancers, liver cancers, colorectal cancers, germ cell tumors, hepatocellular carcinoma, bowel cancers, multiple myeloma, lymphomas (e.g., B cell lymphomas such as diffuse large cell lymphoma), leukemias (e.g., chronic lymphocytic leukemia (CLL), acute lymphoblastic leukemia (ALL), and acute myeloid leukemia (AMIL)), and uveal melanoma. In some cases, a cancer treated as described herein can be a brain stem glioma (e.g., a HGG). For example, a cancer treated as described herein can be a brain stem glioma (e.g., a HGG) in a pediatric human.

In some cases, the materials and methods described herein can be used as a combination therapy with one or more additional agents used to treat a cancer. For example, a mammal in need thereof (e.g., a mammal having cancer) can be administered (a) a population of T cells as described herein, (b) a first antigenic composition as described herein, and (c) a second antigenic composition as a subsequent boost as described herein, in combination with one or more anti-cancer treatments. Examples of anti-cancer treatments that can be used in combination with the administrations of T cell populations and antigenic compositions described herein include, without limitation, cancer surgeries, radiation therapies, chemotherapies (e.g., chemotherapies with alkylating agents such as busulfan), targeted therapies (e.g., GM-CSF inhibiting agents such as lenzilumab), hormonal therapies, angiogenesis inhibitors, immunosuppressants (e.g., interleukin-6 inhibiting agents such as tocilizumab), and cytokine release syndrome (CRS) treatments (e.g., ruxolitinib or ibrutinib). In cases where the materials and methods described herein are used with additional agents treat a cancer, the one or more additional agents can be administered at the same time or independently. In some cases, the materials and methods described herein can be administered first, and the one or more additional agents administered second, or vice versa.

In some cases, the materials and methods described herein can be used to treat a mammal having a disease, disorder, or condition other than cancer. For example, a mammal having a disease, disorder, or condition other than cancer can be administered (a) a population of T cells (e.g., a population of different T cells engineered to each include a CAR) and (b) a first antigenic composition (e.g., a composition including one or more viruses such as one or more oncolytic viruses, a composition including one or more viruses designed to express one or more antigens of interest, and/or a composition including one or more antigenic polypeptides of interest), and after at least about 5 days (e.g., after at least about 7 days, after at least about 10 days, after at least about 14 days, after at least about 20 days, after at least about 50 days, after at least about 60 days, after at least about 75 days, after at least about 3 months, after at least about 4 months, after at least about 5 months, or after at least about 6 months), can be subsequently administered a second antigenic composition (e.g., a composition including one or more viruses such as one or more oncolytic viruses, a composition including one or more viruses designed to express one or more antigens of interest, and/or a composition including one or more antigenic polypeptides of interest). In such cases, the T cells can be designed to express one or more CARs that target an antigen associated with a disease, disorder, or condition. An example of such an antigen that can be targeted by the materials and methods described herein include, without limitation, an urokinase-type plasminogen activator receptor (uPAR) antigen to treat conditions associated with senescence.

The invention will be further described in the following examples, which do not limit the scope of the invention described in the claims.

### EXAMPLES

### Example 1: Dual-Specific CAR T Cells for Cancer Immunotherapy

This Example describes using CAR⁺ T cells and viruses in an immunotherapy to treat cancer. CAR⁺ T cells and viruses were combined ex vivo and systemically delivered to mice having tumors to generate dual-specific, T_{RM} CAR⁺ T cells that can target (e.g., target and destroy) tumor cells and cause tumor regression.

### Materials and Methods

Mice were seeded with tumor. On Day 8 after seeding with tumor, mice were IV administered CAR⁺ T cells alone, virus alone, or a single composition containing CAR⁺ T cells and virus. The CAR⁺ T cells were a population of different T cells having an endogenous TCR repertoire estimated to be greater than 10⁷ different endogenous TCRs that were infected to express a CAR targeting EGFRvIII. The CAR included an scFv specific for EGFRvIII, followed by a CD8 transmembrane domain, followed by a 4-1BB intracellular signaling domain, followed by a CD3ζ intracellular signaling domain (SEQ ID NO:1). IV injections of untransduced T cells were used as control treatments. On Day 15 after seeding with tumor, some mice were IV administered a boost of virus in the absence of CAR⁺ T cells.

To make the single composition containing CAR⁺ T cells and virus, the population of CAR⁺ T cells were loaded with virus by incubating the CAR⁺ T cell population (˜90% CAR positive CD8 T cells) with viral particles (vp) for one hour at 4° C. at a ratio of 10 vp:1 T cell. This process can coat at least some of the CAR⁺ T cells with virus with minimal (e.g., about 5-10 percent) viral infection of the T cells.

### Results

Systemic administration of CAR⁺ T cells loaded with virus resulted in in vivo generation of T_{RM} cells that were dual-specific (e.g., CAR⁺ and positive for an endogenous TCR specific for an antigen of the virus) and that were CD69⁺ and CD103⁺. These dual-specific CAR⁺ T cells were more functional against tumor, persisted longer in vivo than conventional CAR⁺ T cells, and were rapidly re-activated in vivo against tumor by systemic administration of the virus (e.g., a boost), resulting in long-term tumor control.

Melanoma cells were seeded in mice. Eight days after seeding, mice having melanomas were treated via IV injection with CAR⁺ T cells alone, a reovirus alone, or a single composition containing both the CAR⁺ T cells and reovirus. Fifteen days after seeding, mice were boosted via IV injection with reovirus in the absence of T cells. Survival data is shown in Table 1.

TABLE 1
Treatment of melanoma with CAR⁺ T cells and virus.

| | Treatment | Treatment | Survivors |
|---|---|---|---|
| Treatment Group | on Day 8 | on Day 15 | on Day 150 |
| None | Control | Control | 0/7 |
| CAR⁺ T cells alone | CAR⁺ T cells | Control | 1/7 |
| Reovirus alone | Reovirus | Control | 0/8 |
| CAR⁺ T cells/Reovirus | CAR⁺ T cells | Reovirus | 0/8 |
| CAR⁺ T cells + | CAR T cells + | Reovirus | 8/8 |
| Reovirus/Reovirus | Reovirus | | |

Glioma cells were seeded in mice. Eight days after seeding, mice having gliomas were treated via IV injection with CAR⁺ T cells alone, reovirus alone, or a composition including both the CAR⁺ T cells and reovirus. Fifteen days after seeding, mice were boosted via IV injection with either reovirus or VSV as a heterologous virus. Survival data of independent experiments are shown in Table 2 and Table 3.

TABLE 2
Treatment of glioma with CAR⁺ T cells and virus.

| | Treatment | Treatment | Survivors |
|---|---|---|---|
| Treatment Group | on Day 8 | on Day 15 | on Day 60 |
| None | Control | Control | 0/7 |
| CAR⁺ T alone | CAR⁺ T cells | Control | 1/7 |
| Reovirus alone | Reovirus | Control | 0/7 |
| CAR⁺ T + Reovirus/ | CAR⁺ T cells + Reovirus | Reovirus | 7/7 |
| Reovirus | | | |
| CAR⁺ T + Reovirus/ | CAR⁺ T cells + Reovirus | Control | 4/7 |
| Control | | | |
| CAR⁺ T + Reovirus/ | CAR⁺ T cells + Reovirus | VSV | 3/7 |
| VSV | | | |

TABLE 3
Treatment of glioma with CAR⁺ T cells and virus.

| | Treatment | Treatment | Survivors |
|---|---|---|---|
| Treatment Group | on Day 8 | on Day 15 | on Day 45 |
| None | Control | Control | 0/6 |
| CAR⁺ T alone | CAR⁺ T cells | Control | 0/6 |
| Reovirus alone | Reovirus | Control | 0/6 |
| CAR⁺ T/Reovirus | CAR⁺ T cells | Reovirus | 0/6 |
| CAR⁺ T + Reovirus/ | CAR⁺ T cells + Reovirus | Reovirus | 5/6 |
| Reovirus | | | |
| CAR⁺ T + VSV/ | CAR⁺ T cells + VSV | Reovirus | 0/6 |
| Reovirus | | | |

All mice having cancer that were treated with IV injection of CAR⁺ T cells preloaded with virus and then boosted with an IV injection of original treatment virus survived long term compared to other groups.

### Example 2: Persistence of CAR⁺ T Cells

The proportion of total CD8⁺ T cells in the spleen and the blood that were Thy1.1+ve (CAR T marker) and/or VSV specific (VSV tetramer +ve) at day 160 was determined in mice that received either CAR⁺ T cells and VSV-IFNβ or in mice that received CAR⁺ T cells and PBS (FIG. 2).

### Example 3: Persistence of CAR⁺ T Cells

Given the proliferative advantage that dual specific cells have over the rest of the open repertoire, the following was performed to determine if this population also exhibited a functional advantage. CD4⁻ Thy1.1⁺ CAR⁺ T cells were sorted from mice treated according the schedule in FIG. 3A, and they were tested in two functional settings.

In a competitive killing assay at a low effector to target ratio, CAR⁺ T cells isolated from VSV treated mice exhibited more robust killing of target expressing B16EGFRvIII cells over B16 parental cells (FIG. 3B).

In order to discriminate whether the VSV reactive cells were specifically more functional than VSV N ignorant CD8⁺ CAR⁺ T from mice treated with VSV, intracellular staining followed by tetramer staining on CAR⁺ T cell/tumor cell co-cultures were performed. Using the degranulation marker CD107a and the expression of the cytokines IFNγ, TNFα, and IL2, tetramer⁺ cells were found to be both highly functional against their cognate epitope and also more functional against B16EGFRvIII cells, as compared to either tetramer negative cells from the same virus treated animals or sham treated animals (FIG. 3C).

### Example 4: Dual-specific CAR T Cells for Cancer Immunotherapy

### Materials and Methods

Mice were seeded with tumor, and administered compositions containing CAR⁺ T cells as described in Example 1.

CAR⁺ T cells and compositions containing CAR⁺ T cells and virus were prepared as described in Example 1.

### Results

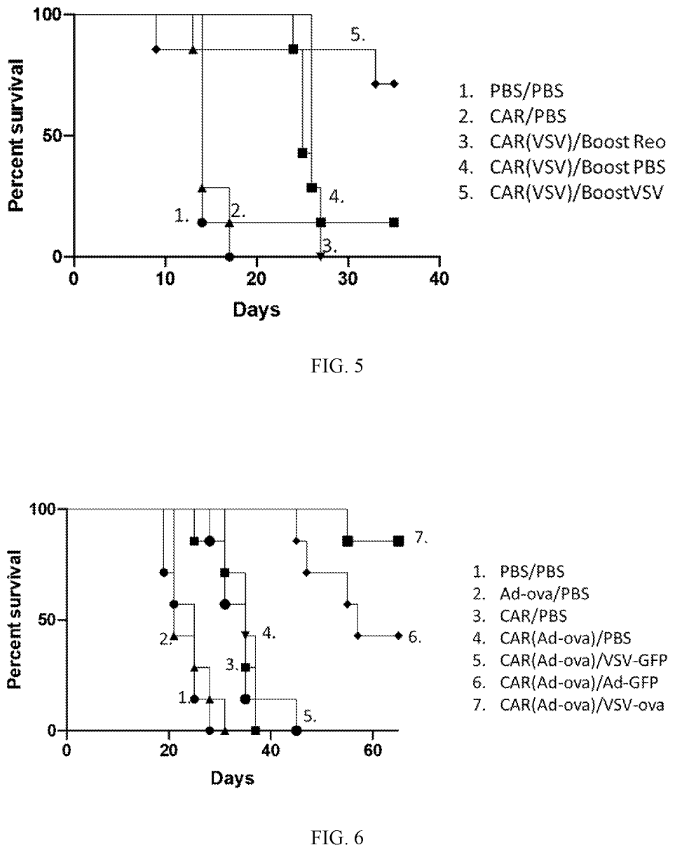

CT2A-EGFRvIII cells were seeded in mice. Eight days after seeding, mice having gliomas were treated with PBS or with 10⁷ anti-EGFRvIII CAR T cells. The CAR T cells were left unloaded or were loaded in vitro with VSV-IFNβ (MOI 1.0, 4° C. for 1 hour). Fifteen days after seeding, mice were boosted with PBS, with 10⁷ pfu of VSV-IFNβ, or 10⁷ pfu of reovirus. Mouse survival with time is shown in FIG. 5. These results demonstrate that CAR T cells loaded with VSV are more effective than CAR T cells alone. These results also demonstrate that CAR T cells loaded with VSV and boosted with homologous VSV are more therapeutic than CAR T alone, CAR T loaded with VSV with no boost, or CAR T loaded with VSV and boosted with a heterologous virus.

B16-EGFRvIII cells were seeded in mice. Eight days after seeding, mice having melanomas were treated with PBS, with 10⁷ pfu of Ad-ova, or with 10⁷ anti-EGFRvIII CAR T cells. The CAR T cells were left unloaded or were loaded in vitro with Ad-ova (MOI 1.0, 4° C. for 1 hour). Nineteen days after seeding, mice were boosted with PBS, with 10⁷ pfu of Ad-ova, with 10⁷ pfu of VSV-GFP, with 10⁷ pfu of VSV-ova, or with 10⁷ pfu of Ad-GFP. Ad-ova and Ad-GFP are replication incompetent adenoviral vectors, serotype 5, expressing the ovalbumin or GFP genes respectively. Mouse survival with time is shown in FIG. 6. These results demonstrate that CAR T cells loaded with Ad can be boosted with Ad to treat cancers. These results also demonstrate that CAR T cells loaded with Ad expressing a heterologous antigen (e.g., a heterologous antigen that is not a tumor-associated antigen) can be boosted with the heterologous antigen (e.g., instead of being boosted with the virus).

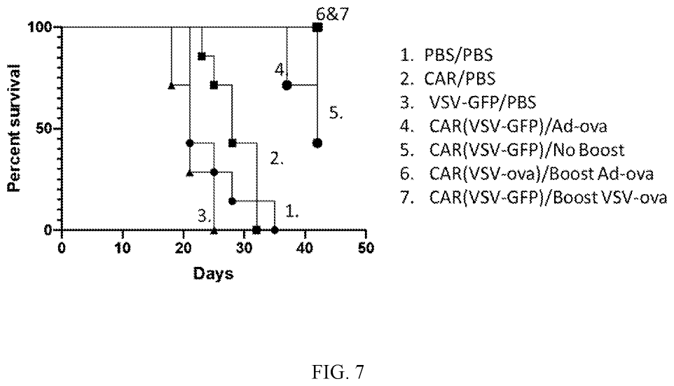

B16-EGFRvIII cells were seeded in mice. Eight days after seeding, mice having melanomas were treated with PBS, with 10⁷ pfu of VSV-GFP, or with 10⁷ anti-EGFRvIII CAR T cells. The CAR T cells were left unloaded or were loaded in vitro with VSV-ova or VSV-GFP (MOI 1.0, 4° C. for 1 hour). Nineteen days after seeding, mice were boosted with PBS, with 10⁷ pfu of Ad-ova, or with 10⁷ pfu of VSV-ova. Mouse survival with time is shown in FIG. 7. These results demonstrate that CAR T cells loaded with VSV can be more effective than CAR T cells alone or VSV alone. These results also demonstrate that CAR T cells loaded with VSV can be boosted with VSV to treat cancers. These results also demonstrate that CAR T cells loaded with VSV expressing a heterologous antigen (e.g., a heterologous antigen that is not a tumor-associated antigen) can be boosted with the heterologous antigen (e.g., instead of being boosted with the virus).

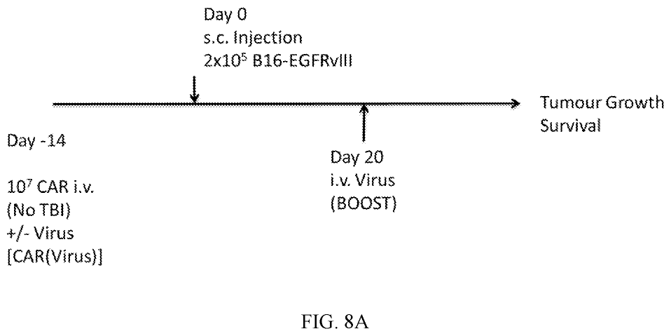

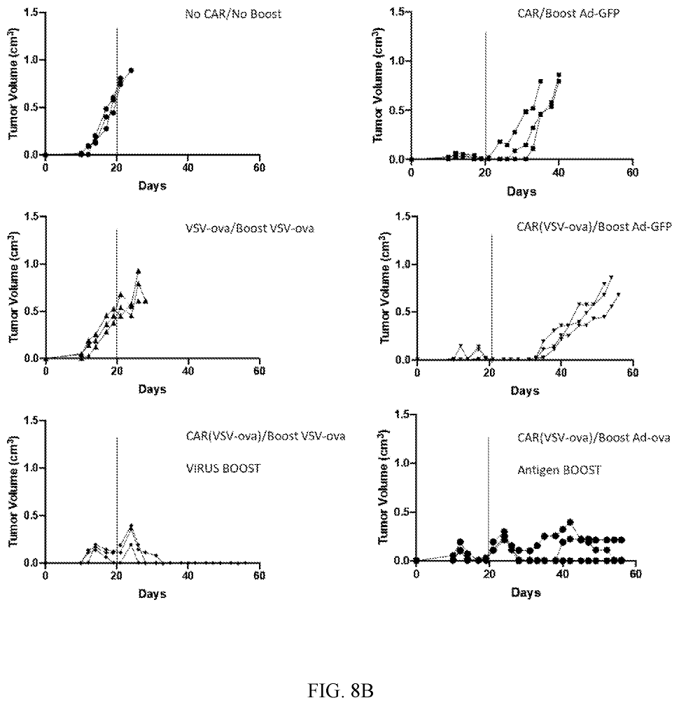

Mice were treated as shown in FIG. 8A. On day −14, mice were administered PBS, 10⁷ pfu VSV-ova, or 10⁷ anti-EGFRvIII CAR T cells. The CAR T cells were left unloaded or were loaded in vitro with VSV-ova (MOI 1.0, 4° C. for 1 hour). On day 0, mice were injected subcutaneously with 2×10⁵ B16-EGFRvIII tumor cells. On day 20, mice were boosted with PBS, with 10⁷ pfu of Ad-GFP, with 10⁷ pfu of Ad-ova, or with 10⁷ pfu of VSV-ova. Tumor volume is shown in FIG. 8B. These results demonstrate that a pre-existing load of dual specific CAR T cells in vivo can be re-activated by a boost with virus or antigen to treat subcutaneous tumors, and suggests that dual specific CAR T cells can be used to treat recurrent disease.

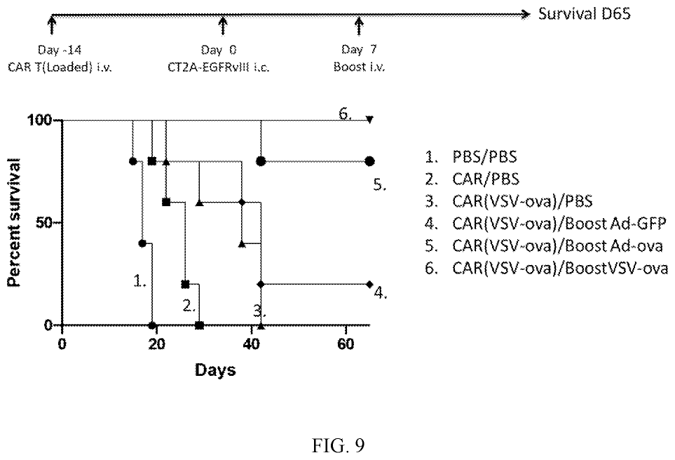

Mice were treated as shown in the timeline in the top panel of FIG. 9. On day −14, mice were administered PBS or 10⁷ anti-EGFRvIII CAR T cells. The CAR T cells were left unloaded or were loaded in vitro with VSV-ova (MOI 1.0, 4° C. for 1 hour). On day 0, mice were injected intra-cranially into the brainstem with 2×10⁴ CT2A-EGFRvIII tumor cells. On day 7, mice were boosted with PBS, with 10⁷ pfu of Ad-GFP, with 10⁷ pfu of Ad-ova, or with 10⁷ pfu of VSV-ova. Mouse survival time is shown in FIG. 9 (bottom panel). These results demonstrate that a pre-existing load of dual specific CAR T cells in vivo can be re-activated by a boost with virus or antigen to treat intra-cranial brain tumors. These results also demonstrate that virus-loaded CAR T cells can be used prophylactically to prevent tumor development and/or tumor recurrence in the brainstem.

B16-EGFRvIII cells were seeded in mice. Eight days after seeding, mice having melanomas were treated with 10⁶ anti-EGFRvIII CAR T cells loaded in vitro with reovirus (MOI 1.0, 4° C. for 1 hour). Fifteen days after seeding, mice were boosted with 10⁷ pfu reovirus. Out of a group of 15 mice, 3 developed recurrent tumors. Once a mouse developed a palpable recurrence, it was administered an i.v. injection of 10⁷ pfu of reovirus, VSV-GFP or PBS (arrow). Tumor volume is shown in FIG. 10. These results demonstrate that recurrent tumors can be treated with a further boost with homologous virus.

B16-EGFRvIII cells were seeded in mice. Eight days after seeding, mice having melanomas were treated with PBS, with 10⁷ pfu of reovirus, or with 10⁷ anti-EGFRvIII CAR T cells. The CAR T cells were left unloaded or were loaded in vitro with reovirus (MOI 1.0, 4° C. for 1 hour). Fifteen days after seeding, mice were boosted with PBS, with 10⁷ pfu of VSV-GFP, or with 10⁷ pfu of reovirus. Mouse survival time is shown in FIG. 11. These results demonstrate that reovirus-loaded CAR T cells are more therapeutic than unloaded Car T cells. These results also demonstrate that reovirus-loaded CAR T cells are highly therapeutic when boosted in vivo with reovirus.

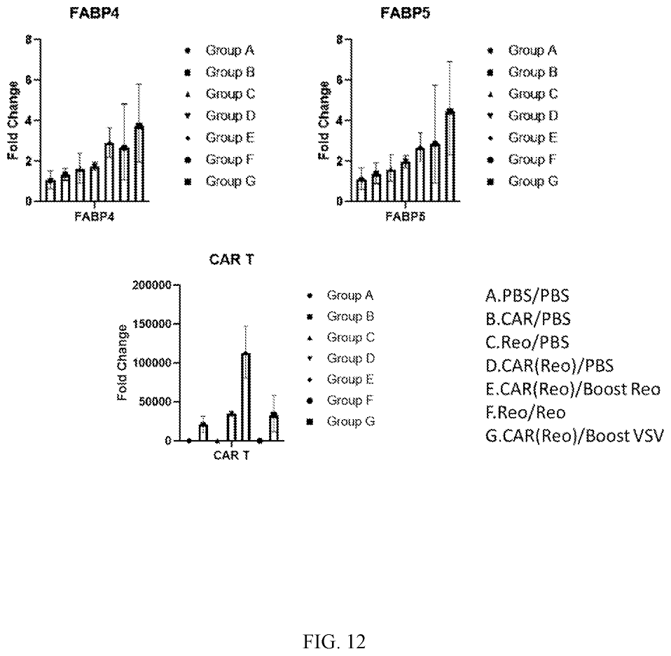

RNA was extracted from skin/tumor samples excised from mice treated as described for FIG. 11. The levels of FABP4 and FABP5 (markers for T_{RM} cells) and the levels of the CAR specific retroviral vector were measured by qrtPCR (FIG. 12). These results demonstrate that T_{RM} can persist at the site of tumor at high level in mice treated with reovirus-loaded CAR T cells and boosted with homologous or heterologous virus. These results also demonstrate that CAR T cells can persist at the site of tumor at high levels in mice treated with reovirus-loaded CAR T cells and boosted with homologous virus.

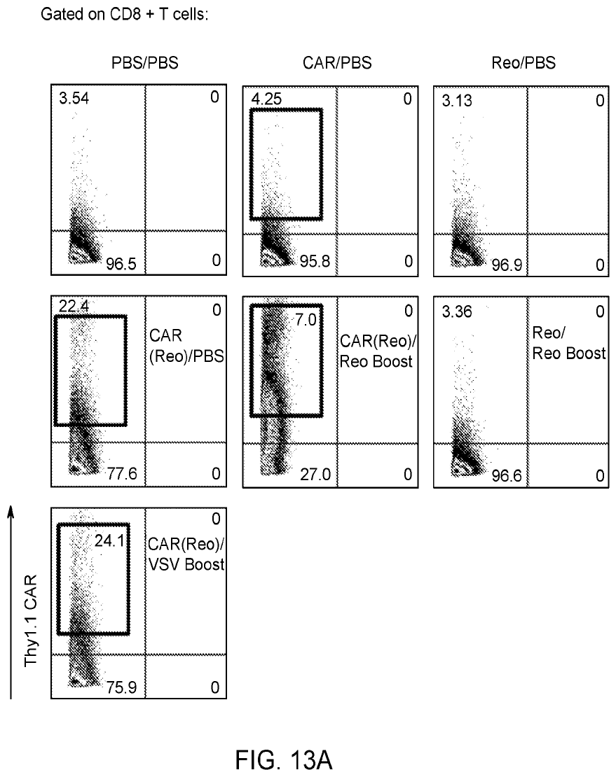

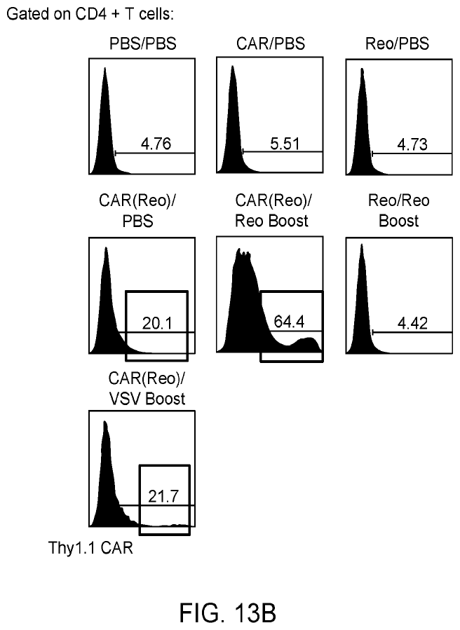

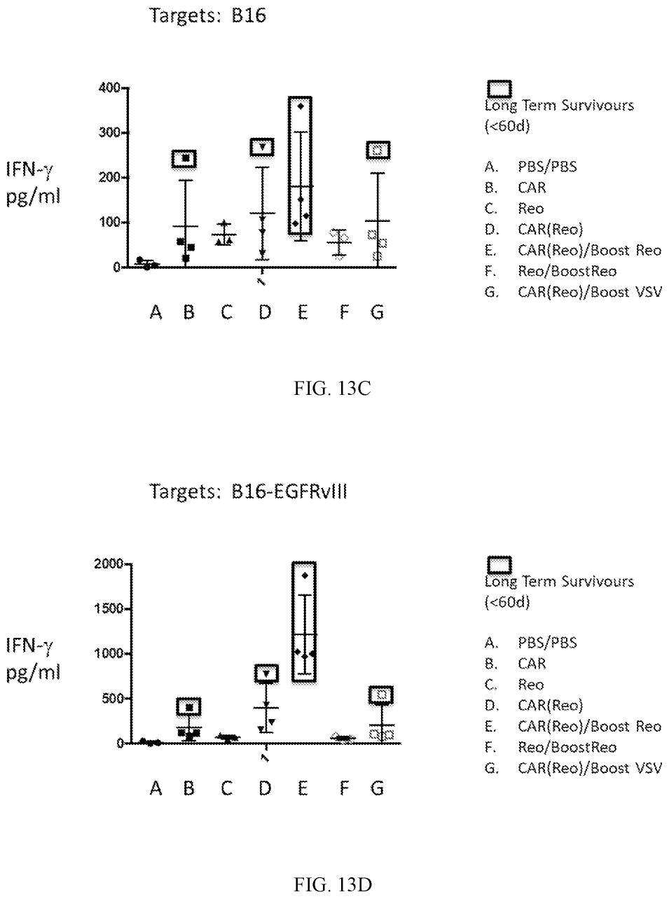

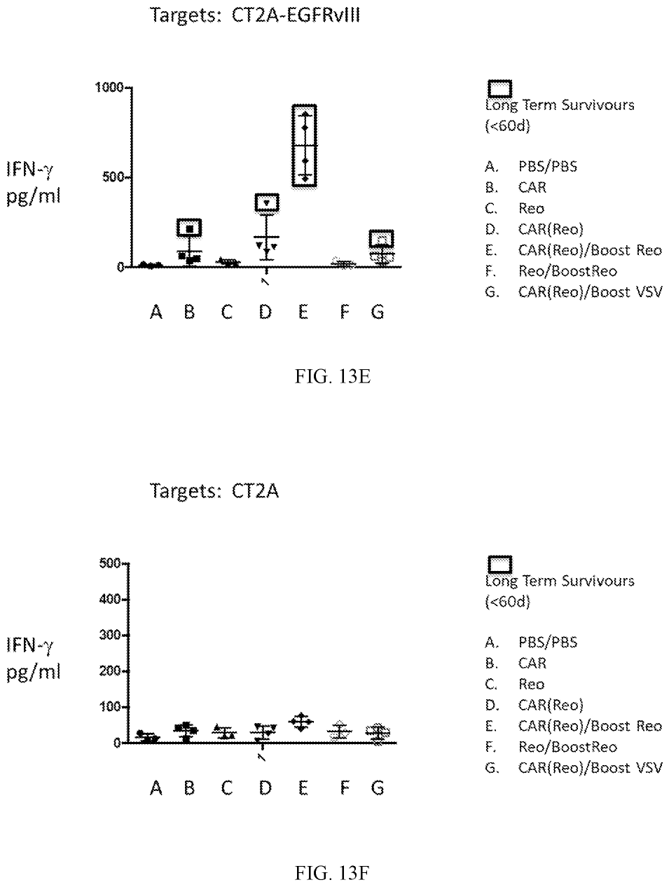

Mice were treated as described for FIG. 11. At the time of euthanasia due to tumor size, splenocytes were harvested from 3 mice per treatment group and pooled together. The proportion of total CD8⁺ T cells in the spleen that were Thy1.1+ve (CAR T marker) was determined in mice for each treatment group (FIG. 13A), and the proportion of total CD4⁺ T cells in the spleen that were Thy1.1+ve (CAR T marker) was determined in mice for each treatment group (FIG. 13B). These results demonstrate that CAR T cells alone generate poor persistence of CAR T cells while CAR T cells loaded with reovirus generate a persistent population of CAR T cells. These results also demonstrate that CAR T cells loaded with reovirus and a boost of reovirus generates an excellent population of persistent CAR T cells. The pooled splenocytes from each of the treatment groups were also co-cultured with target B16, B16-EGFRvIII, CT2A-EGFRvIII, or CT2A tumor cells at an Effector:Target ratio of 10:1. 72 hours later levels of IFN-7 in the supernatants were measured by ELISA (FIGS. 13C-13F). These results demonstrate that T cells that are reactive against B16 tumors persist in long term survivors. These results also demonstrate that EGFRvIII-reactive T cells persist in mice treated with reovirus-loaded CAR T cells and boosted with reovirus.

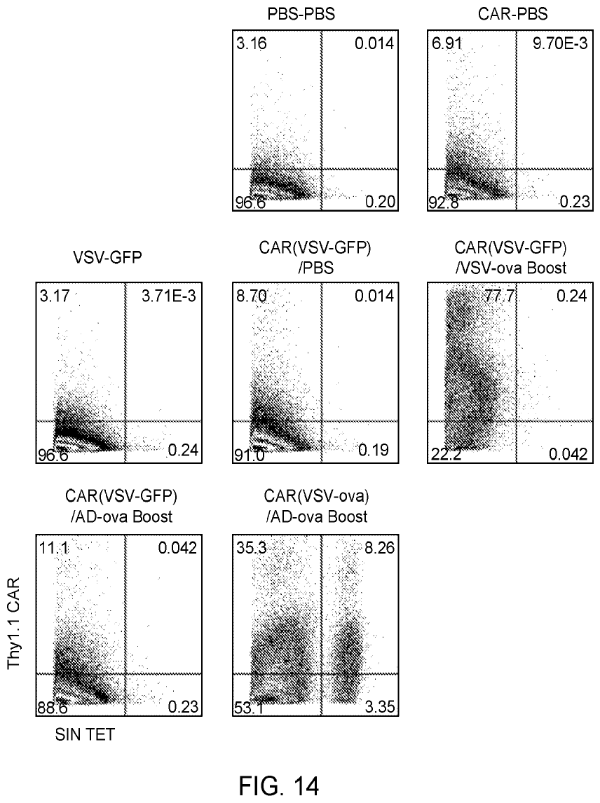

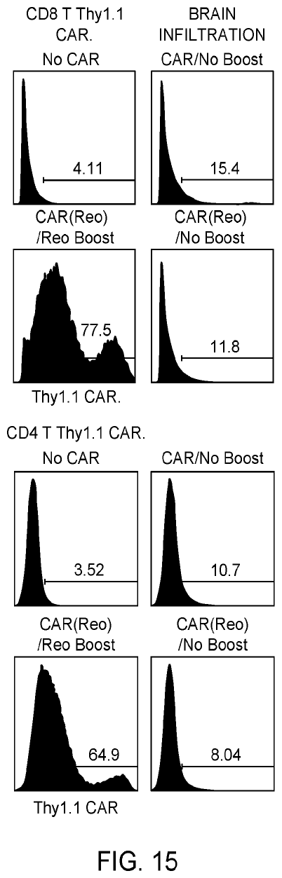

B16-EGFRvIII cells were seeded in mice. Eight days after seeding, mice having melanomas were treated with PBS, with 10⁷ pfu of VSV-GFP, or with 10⁷ anti-EGFRvIII CAR T cells. The CAR T cells were left unloaded or were loaded in vitro with VSV-GFP or VSV-ova (MOI 1.0, 4° C. for 1 hour). Fifteen days after seeding, mice were boosted with PBS, with 10⁷ pfu of Ad-ova, or with 10⁷ pfu of VSV-ova. At the time of euthanasia due to tumor size, splenocytes were harvested from 3 mice per treatment group and pooled together. The proportion of total CD8⁺ T cells in the spleen that were single specific CAR (Thy1.1+ve), single specific OVA (SIINFEKL tetramer (SEQ ID NO:5)+ve), or dual specific (Thy1.1+, SIINFEKL tetramer (SEQ ID NO:5)+ve) was determined in mice from each treatment group (FIG. 14). CAR T cells persist at very low levels (6.91 wrt 3.16). Loading of CAR with VSV enhances CAR T cell persistence (8.77 wrt 3.16). Boosting in vivo with homologous virus increases CAR T cell persistence and numbers (77.7 wrt 3.16). Boosting in vivo with heterologous virus can increase CAR T cell persistence (11.1 wrt 8.77). Boosting in vivo with a heterologous virus but a homologous virus-encoded antigen generates persistence of CAR T cells, antigen-specific CD8⁺ T cells, and dual specific CAR T cells. These results demonstrate that administering CAR T cells loaded with virus and an in vivo boost can lead to persistence of high levels of dual specific CAR T cells.

CT2A-EGFRvIII cells were seeded in mice. Eight days after seeding, mice having gliomas were treated with PBS or with 10⁷ anti-EGFRvIII CAR T cells. The CAR T cells were left unloaded or were loaded in vitro with reovirus (MOI 1.0, 4° C. for 1 hour). Fifteen days after seeding, mice were boosted with PBS or 10⁷ pfu of reovirus. At the time of euthanasia, brains were harvested from 3 mice per treatment group and pooled together. The proportion of total CD8⁺ T cells in the brain that were Thy1.1+ve (CAR T marker) was determined in mice from each treatment group (FIG. 14). These results demonstrate that administering CAR T Cells loaded with virus and providing an in vivo boost can lead to infiltration of CAR T cells into the brain.

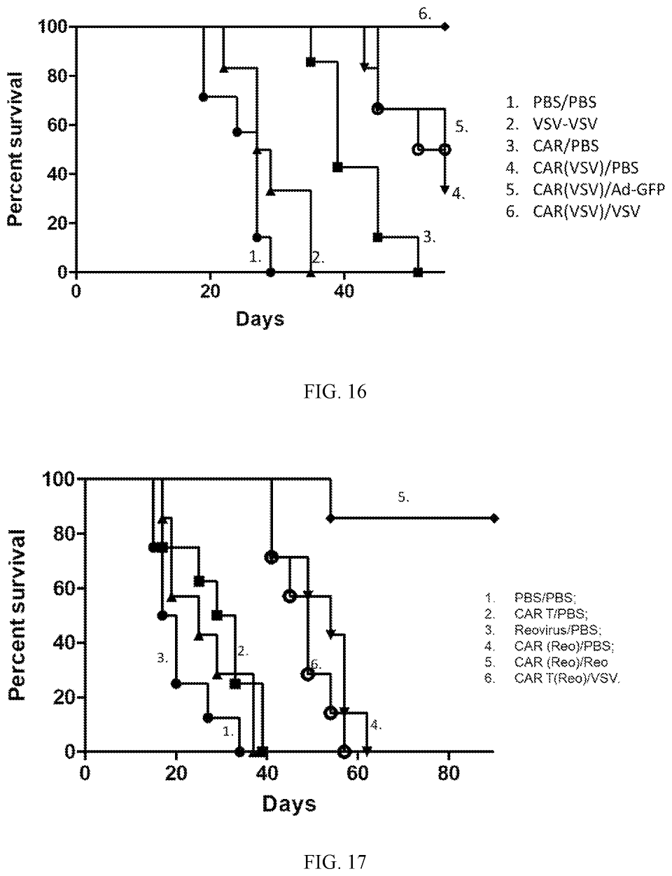

B16-CD19 cells were seeded in mice. Eight days after seeding, mice having melanomas were treated with PBS, with 10⁷ pfu of VSV-IFNβ, or with 10⁷ anti-CD19 CAR T cells. The CAR T cells were left unloaded or were loaded in vitro with VSV-IFNβ (MOI 1.0, 4° C. for 1 hour). Fifteen days after seeding, mice were boosted with PBS, with 10⁷ pfu of Ad-GFP, or with 10⁷ pfu of VSV-IFNβ. Mouse survival time is shown in FIG. 16. These results demonstrate that dual specific CAR T cells designed using multiple types of CAR T cells.

CT2A-EGFRvIII cells were seeded in mice. Eight days after seeding, mice having frontal lobe tumors were treated day 8 and boosted on day 15 with: 1. PBS/PBS; 2.CAR T/PBS; 3. reovirus/PBS; 4. CAR T loaded with reovirus/PBS; 5. CAR T loaded with reovirus/reovirus; or 6. CAR T loaded with reovirus/VSV. Mouse survival time at day 90 is shown in FIG. 17. These results demonstrate that dual specific, re-activatable CAR T cells can treat intra-cranial tumors in the frontal lobe as well as in the brainstem.

### Example 5: Oncolytic Virus-Mediated Expansion of Dual Specific Dual-Purpose CAR T Cells with Improved Efficacy Against Solid Tumors

This Example describes a new mechanism by which OVs can potentiate CAR T efficacy against solid tumors in which stimulation of the native TCR gives rise to enhanced proliferative and functional properties and distinct memory phenotypes. In vivo expansion of dual specific CAR T was leveraged by in vitro pre-loading with antigenic compositions (e.g., oncolytic Vesicular Stomatitis virus (VSV) or reovirus), allowing for a further in vivo expansion/re-activation by homologous boosting, and led to high cure rates in tumors located in multiple anatomical sites. Thus, this Example shows that stimulation of the native TCR can be exploited to enhance CAR T cell activity and efficacy in vivo. The results in this Example re-present and expand on the results provided in other Examples.

### Results

Combination of CAR T Cells with Oncolytic VSV Primes Virus-Specific CAR T Cells In Vivo

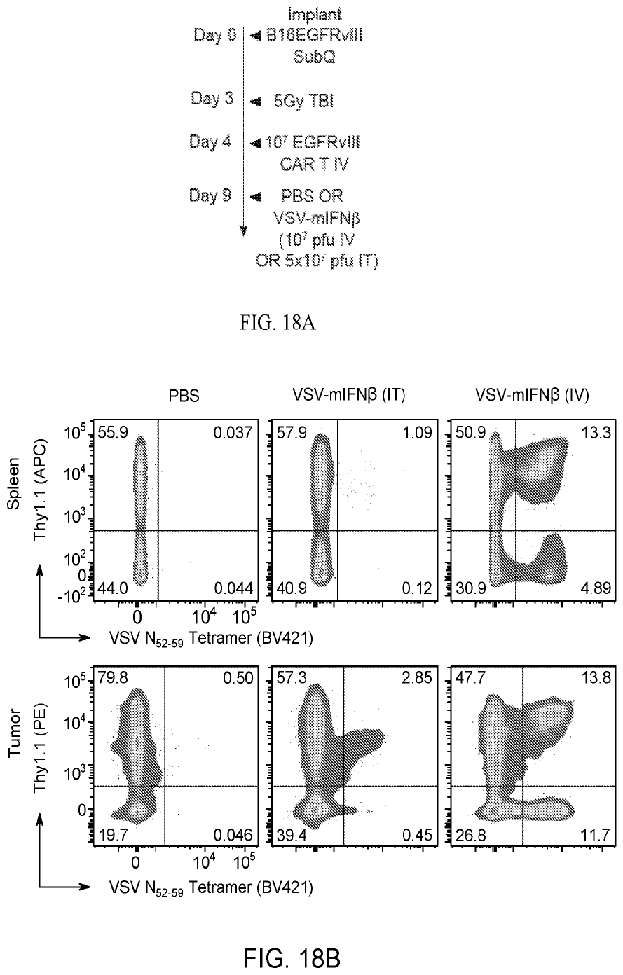

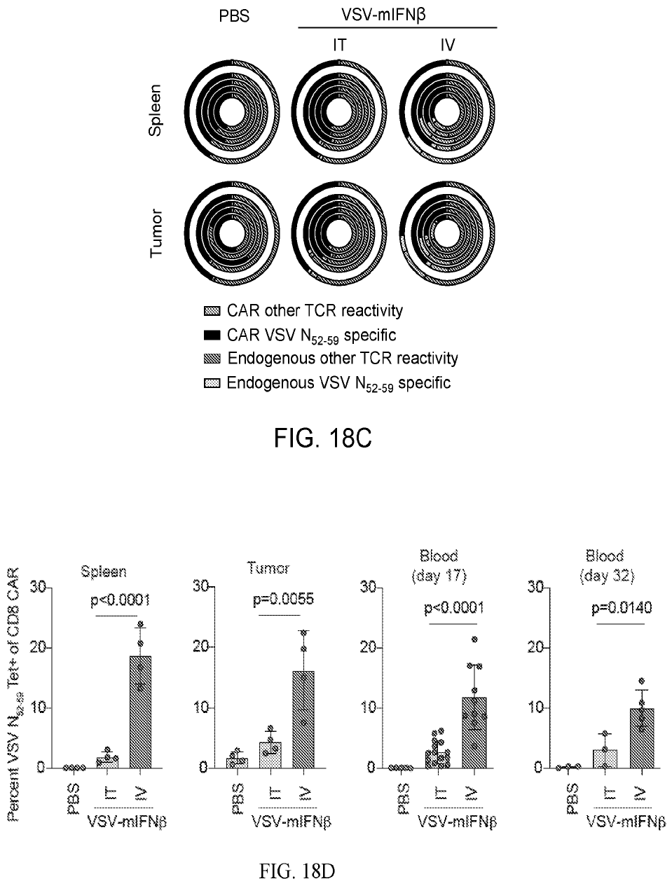

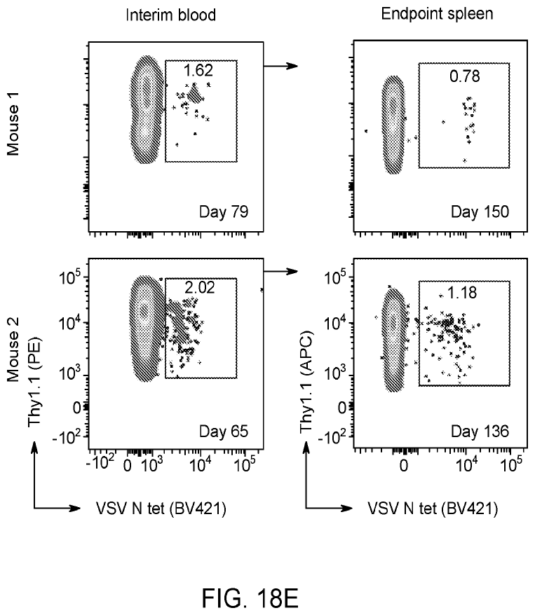

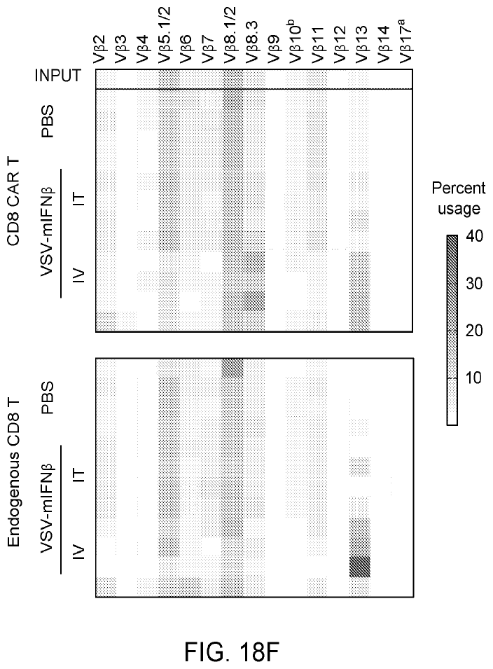

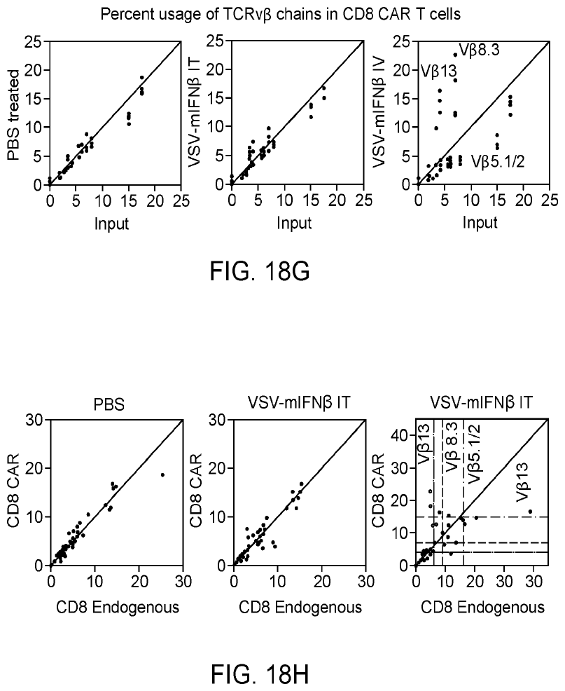

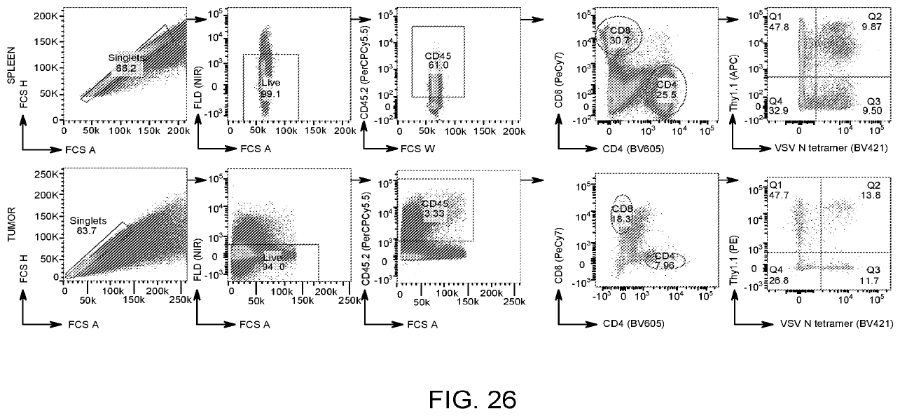

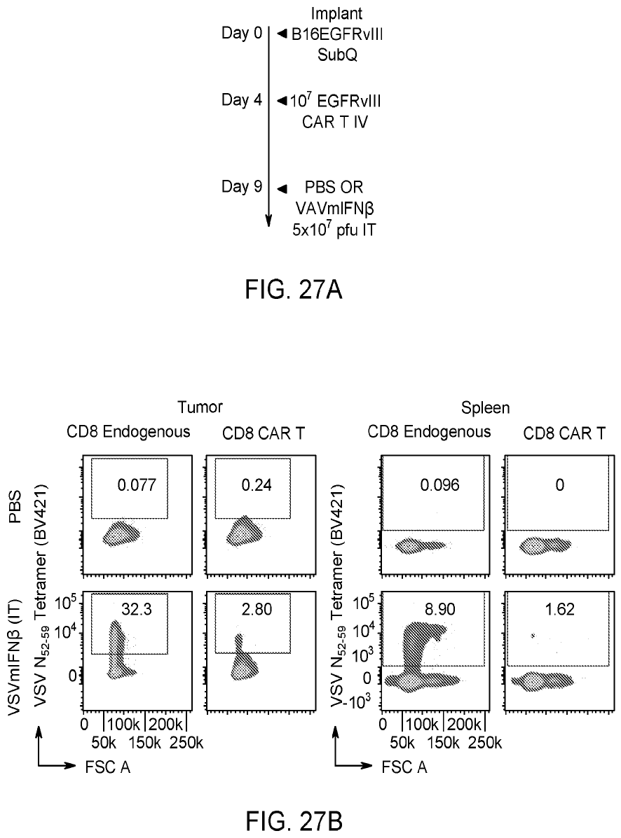

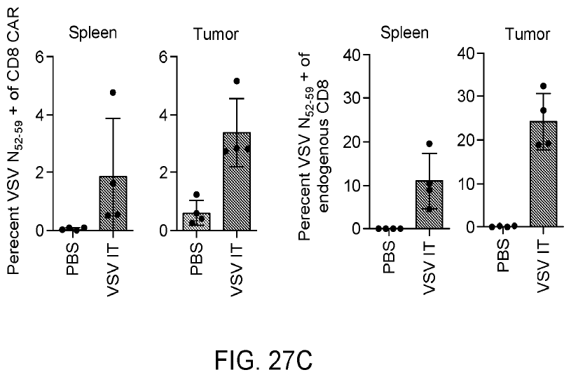

Using a schedule where administration of CAR T cells was followed 5-6 days later by either an IT or intravenous (IV) dose of VSV-mIFNβ, CAR T attrition was not observed in the tumor, spleen, or blood of virus treated animals (FIG. 25). However, a highly significant, selective expansion of CD8 CAR T with native TCR specificity to the immunodominant epitope VSV N₅₂₋₅₉ was observed (FIGS. 18A-18C). VSV-N52-59-specific CAR T cells were detected following both IT and IV virus administration, however, the expansion was greater in animals that received IV virus (FIG. 18D). A sample gating scheme is shown in FIG. 26. The proportion of dual specific CAR T cells was greatest 7 days post virus exposure and, although it contracted over time, was still detectable in the blood and spleen over 100 days post-transfer (FIG. 18E). The expansion of dual specific CD8 CAR T was enhanced by lymphodepletion. In non-preconditioned mice, although a proportionally similar expansion of virus specific CD8 CAR T was observed, numerically, this was very low due to limited CAR T engraftment. The endogenous VSV N-specific CD8 population was significantly larger (FIG. 27).

The CAR T cells generated in FIG. 18 were derived from an open repertoire of unselected T cells from naïve mice, in which the reported precursor frequency of T cells with TCR specificity for H2-K^{b} restricted VSV N₅₂₋₅₉ is ˜8.24×10⁻⁴% of CD8 T cells. Assuming that the CAR T product is ˜70% CD8 cells, from a dose of 10⁷ cells, approximately 50 T cells would have had specificity for the VSV-N₅₃₋₅₉ epitope. Despite this low predicted frequency in the input product, a subsequent expansion of this dual specific population to ˜10-20% of the CD8 CAR T cell population was observed in vivo (FIG. 18).

To evaluate how virus administration alters the clonality of the CAR T population, a panel of antibodies recognizing commonly used TCR variable beta (VO) chains was used to profile the injected CAR T, as well as those cells recovered from mice treated with PBS or VSV-mIFNβ (IT or IV). The repertoire of Vβ chain usage of the CAR T cell population from animals which received PBS was very similar to the injected CAR T cells (FIGS. 18F and 18G). Following IV VSV-mIFNβ, the profile of TCR Vβ usage was significantly shifted from that of the input population—with a high degree of selection for CAR T cells using the Vβ13 and Vβ8.3 chains, as well as a reduced utilization of Vβ5/1.2 (FIGS. 18F and 18G). Consistent with the tetramer staining showing that IT administration of virus induced a more modest expansion of dual specific CAR T cells, low level dynamic changes were observed in the Vβ chain usage in mice that received IT virus. In animals treated with IV VSV-mIFNβ, a similar selection of usage of the Vβ13 TCR was observed in the endogenous CD8 T cell population (FIG. 18H).

### Dual Specific (DS) CAR T Cells Acquire Distinct Functional and Phenotypic Properties

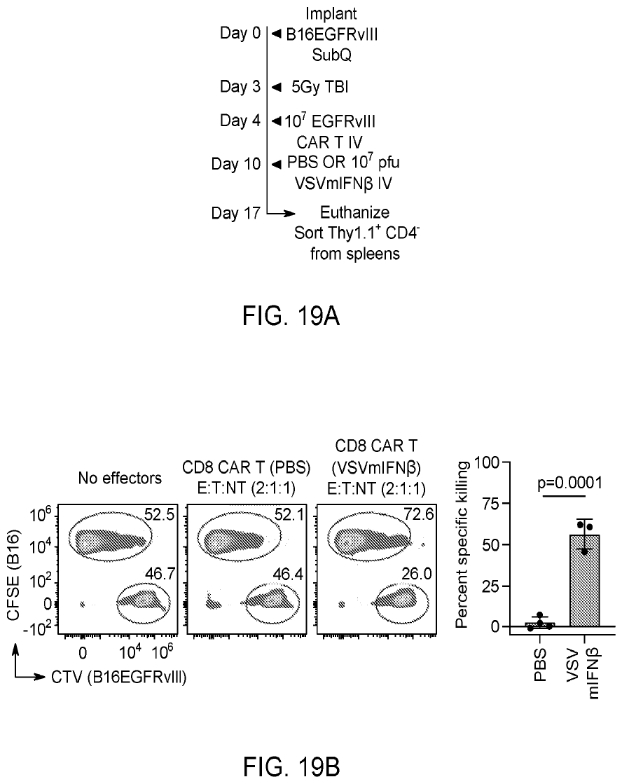

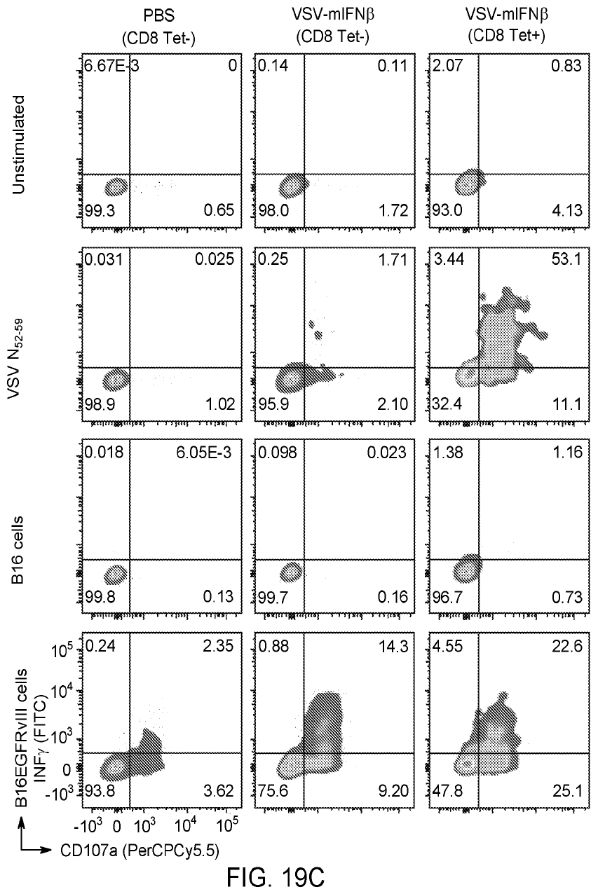

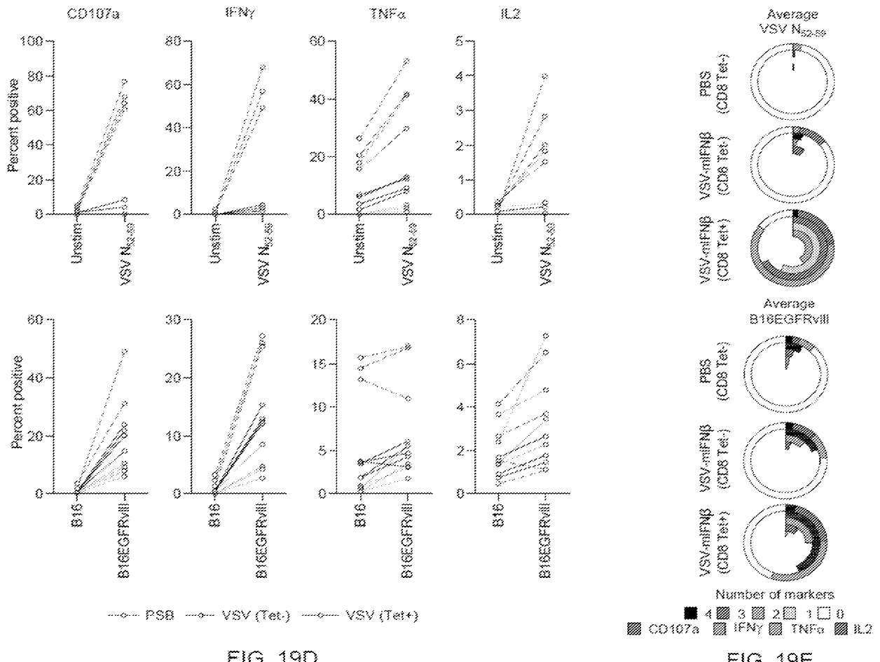

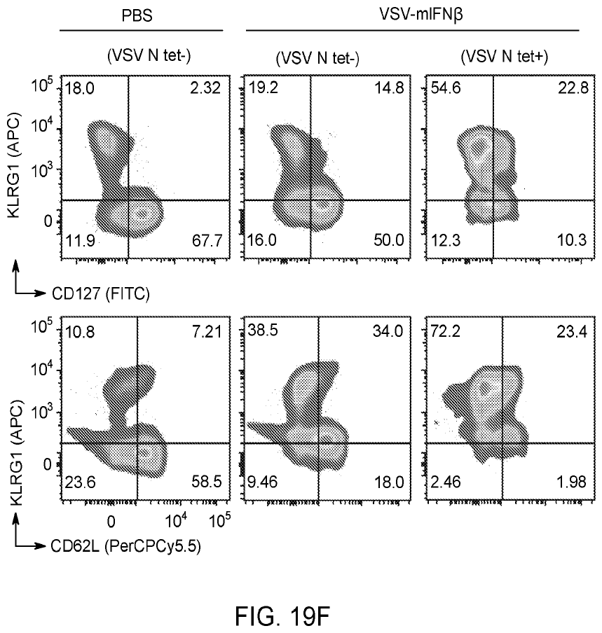

The fact that the virus-specific dual specific CAR T cells could reach up to 25% of the surviving CAR T cell population in vivo indicated that there was a very strong selective advantage to the expansion and survival of these cells over those with no adjunct TCR signaling/activation. In addition to this proliferative advantage, sorted CD8 CAR T isolated from mice co-administered with CAR T cells and VSV-IFNβ exhibited significantly more robust killing of B16EGFRvIII cells over B16 parental cells in vitro (FIGS. 19A and 19B). In contrast, at a low effector:target:non-target ratio of 2:1:1, CAR T cells recovered from mice treated with PBS showed low levels of specific killing of EGFRvIII expressing tumor cells.

Both dual specific and VSV N ignorant CD8 CAR T from virus treated animals would have been exposed to the same inflammatory environment, and therefore to more specifically interrogate whether the dual specific CAR T were more active, degranulation and cytokine expression were examined in VSV N₅₂₋₅₉ tetramer-stained samples. When stimulated with the VSV N₅₂₋₅₉ peptide, highest levels of CD107a expression, and IFNγ, TNF-α production were observed in the VSV N₅₂₋₅₉ tetramer-stained population (FIGS. 19C, 19D, and 19E). In addition, when sorted CD8 CAR T were co-cultured with B16EGFRvIII cells, the highest levels of degranulation and cytokine production were observed from the tetramer positive population. The tetramer negative population from virus treated animals underwent enhanced degranulation, and expressed higher levels of IFNγ than the CD8 CAR T from PBS treated animals.

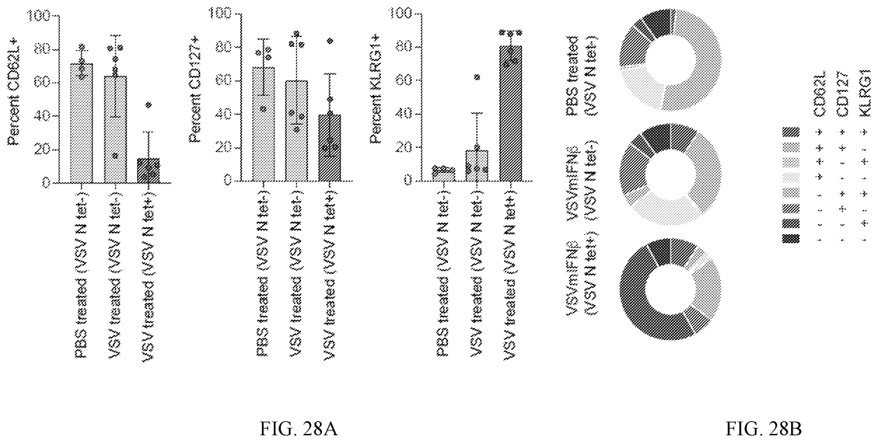

Consistent with these functional differences, VSV N₅₂₋₅₉-specific CD8 CAR T cells had a KLRG1^{hi}, CD127^{lo}, CD62L^{lo} effector memory phenotype, which was significantly different to that of VSV N ignorant CD8 CAR T from virus treated mice, and CD8 CAR T from PBS treated mice (FIGS. 19F and 19G). Nonetheless, the KLRG1, CD62L, and CD127 profiles among the CD8 CAR T cells from PBS and virus treated animals were very similar to the corresponding endogenous CD8 T cell populations (FIG. 28).

Taken together, these results demonstrate that the strong signaling through the TCR resulting from MHC presentation of viral antigens to an open TCR repertoire of CAR T cells leads to a powerful selective survival and proliferative advantage. Moreover, this signaling is associated with improved function against the CAR antigen and an altered memory differentiation profile.

Fully Systemic Tumor Treatment with Ex Vivo Viral Loaded CAR T Cells Expands Dual Specific CAR T in Non-Preconditioned Animals

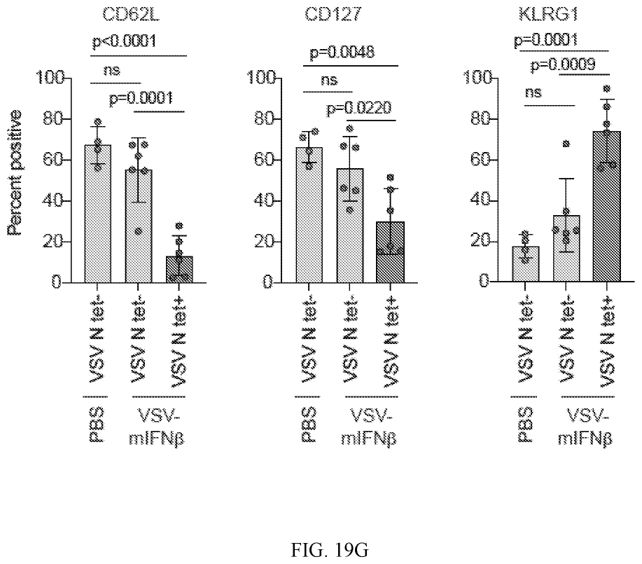

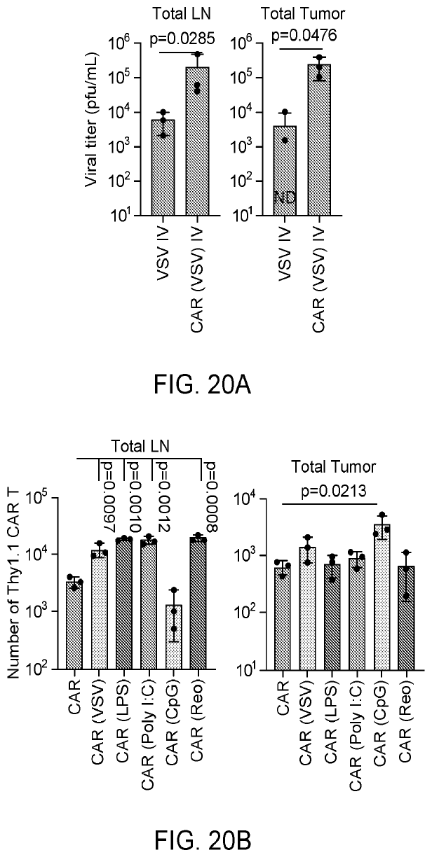

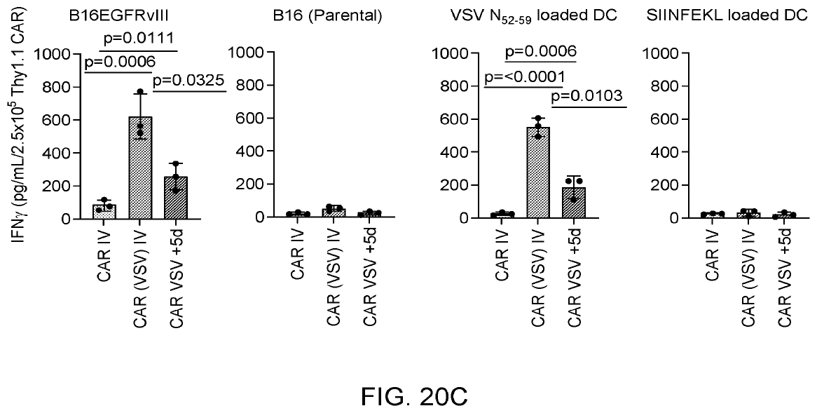

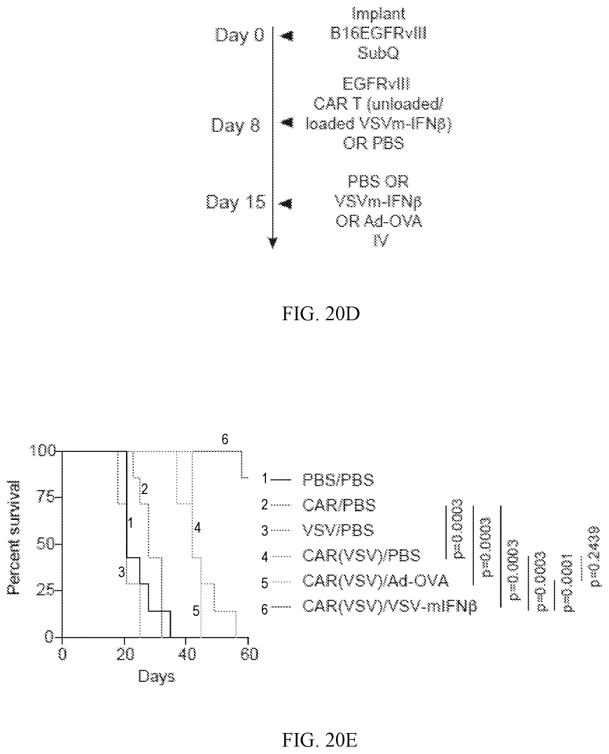

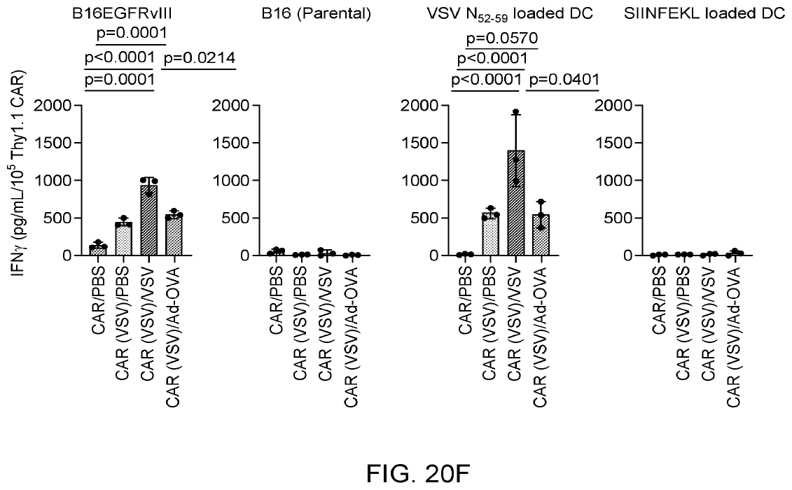

It was examined whether the generation of dual specific CAR T cells could be optimized by enhancing the levels of co-delivery of both virus and CAR T cells to secondary lymphoid organs for presentation of viral antigens to the CAR T cells. Although IV administered VSV was readily detected in the LN, significantly higher levels were detected following the ex vivo loading of CAR T cells with the virus (FIG. 20A). This in vitro loading of CAR T cells with virus also led to significantly increased levels of VSV delivery to subcutaneous tumors compared to IV delivery alone. In addition to improved viral delivery to LNs and tumors, greater numbers of CAR T were recovered from LNs if cells had been pre-loaded with VSV in vitro (FIG. 20B). The improved LN localization of virus loaded CAR T cells was mimicked by pre-treatment of CAR T cells with LPS or polyIC, but not CpG, suggesting that virus loading may be triggering CAR T cell activation and altered trafficking through Toll Like Receptors 3 or 4 (but not 9) (FIG. 20B). Neither virus loading, nor LPS or poly(I:C) treatment, of CAR T cells significantly enhanced CAR T cell trafficking to the tumor, although prior treatment with CpG was able to increase CAR T cell levels in the tumor by about 3-fold (FIG. 20B). Very similar effects were observed by loading the CAR T cells in vitro with oncolytic reovirus (FIG. 20B).

Consistent with the results in FIG. 19, dual specific CAR T cells generated by in vitro CAR T cell loading were also significantly more functional than CAR T administered as a monotherapy. CAR T cells isolated from splenocytes of mice treated 15 days previously with CAR T cells alone secreted low but detectable levels of IFNγ when co-cultured with B16EGFRvIII target cells, but not when co-cultured with parental B16 cells (FIG. 20C). CAR T cells from mice treated with VSV-mIFNβ 5 days following CAR T transfer were significantly more active against B16EGFRvIII targets than CAR from mice treated with no virus, and were indeed dual specific as measured by reactivity against the VSV-N₅₂₋₅₉ peptide (FIG. 20C). However, CAR T cells recovered from mice treated with in vitro VSV-mIFNβ loaded CAR T secreted significantly more IFNγ in response to both CAR antigen and VSV antigen, than CAR T from either of the other two groups (FIG. 20C). Taken together, these results demonstrate that in vitro loading of CAR T cells with an antigenic composition such as a virus enhanced both CAR T and antigenic composition delivery (e.g., virus delivery) to the LN (for priming of dual specific CAR T cells), enhanced virus delivery to tumors (for direct oncolysis), and was a more effective way to generate dual specific CAR T cells in vivo in non-preconditioned mammals than by physically and temporally separating CAR T and virus administration.

Since in vitro loading of CAR T with VSV could enhance virus delivery to tumors, and promote the expansion of dual specific CAR T with greater function, whether this strategy could be further exploited therapeutically was examined. In particular, a second in vivo boost with virus was used to re-stimulate memory CAR T with viral TCR specificity. Consistent with the enhanced function of dual specific CAR T over CAR T alone, in vitro viral loaded CAR T cells conferred a significant survival advantage compared to either unloaded CAR T cells or IV virus alone against subcutaneous B16EGFRvIII tumors (FIGS. 20D and 20E). However, treatment with virus loaded CAR T cells alone was insufficient to cure any mice long term in this regimen. In contrast, a boost with IV VSV-mIFNβ was able to re-stimulate dual specific CAR T cell activity in vivo leading to long term cures in the majority of mice. Anti-tumor efficacy was dependent upon re-stimulation of VSV-specific CAR T cells because a boost with a different virus expressing an irrelevant antigen (Ad-OVA) was no more effective than treatment with VSV-loaded CAR T cells with no boost. CAR T cells recovered from spleens of mice treated with virus loaded CAR T cells were significantly more active against B16EGFRvIII targets than were CAR T from mice treated with CAR T cells alone (FIG. 20) (spleens recovered either at euthanasia due to tumor size, or at the termination of the experiment at day 60). However, a boost with VSV-mIFNβ, but not with a heterologous virus (Ad-OVA), significantly enhanced the activity of the CAR T cells against B16EGFRvIII targets. Reactivity of these CAR T cells against the immunodominant VSV-N₅₂₋₅₉ peptide mirrored the reactivity against the EGFRvIII targets confirming their dual specificity following co-administration of CAR T with virus in vivo. Taken together, these results demonstrate that the in vivo generation of dual specific CAR T cells can be exploited therapeutically in a fully systemic treatment regimen through the boosting of memory virus-specific CAR T cells, thereby provided highly effective immunotherapy treatments for cancer.

### Virus Loading and Boosting Dual Specific CAR T Cell Therapy is not Dependent Upon Virus or Tumor Type

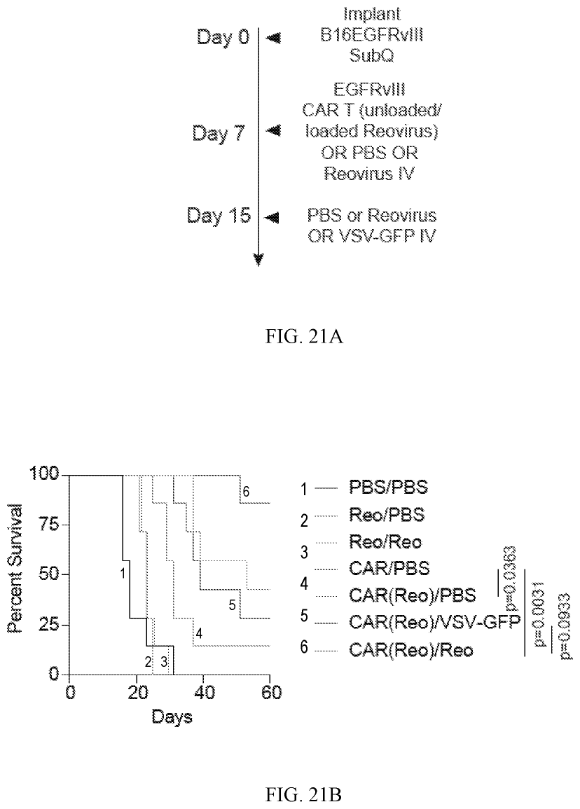

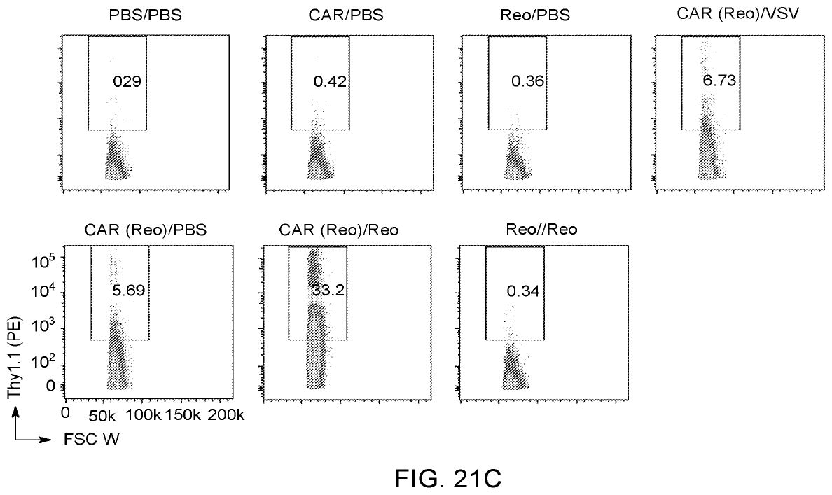

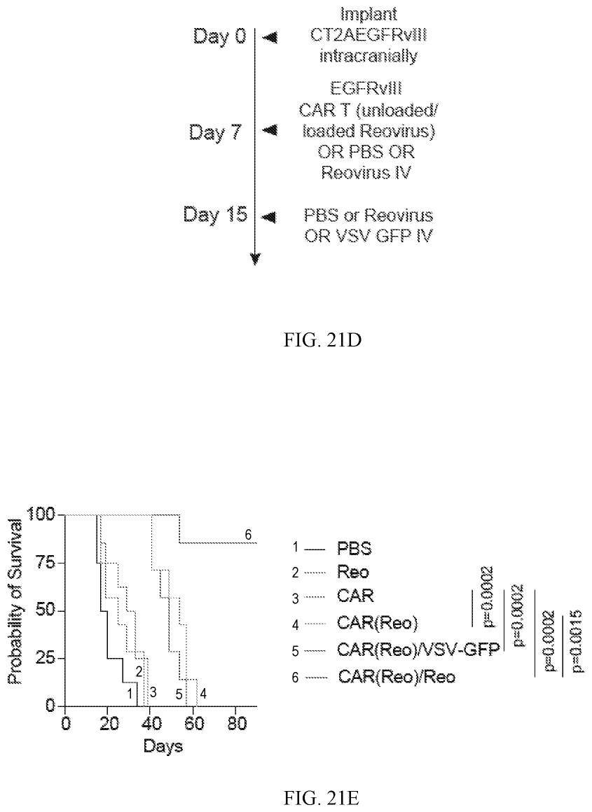

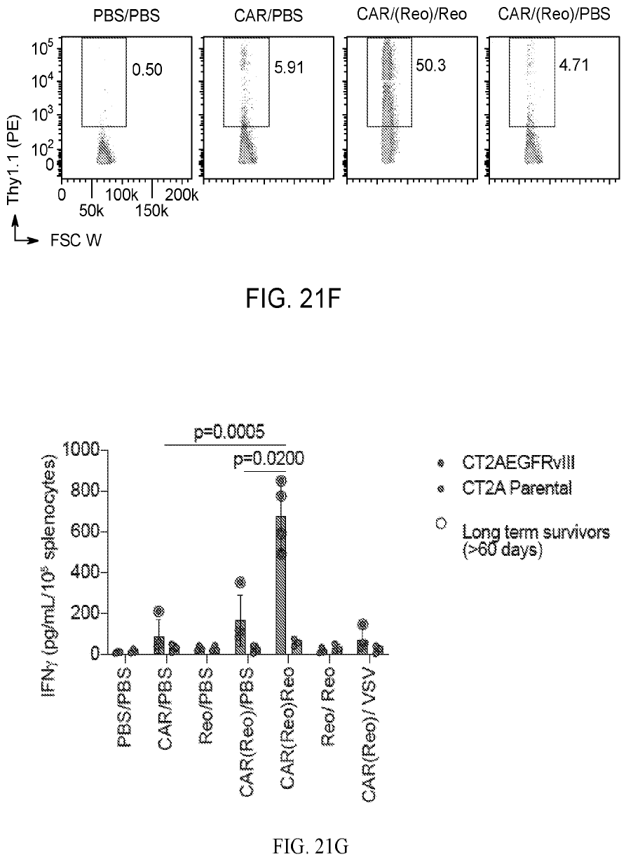

CAR T cells pre-loaded with oncolytic reovirus led to significantly increased therapy compared to unloaded CAR T cells in the same subcutaneous B16EGFRvIII model (FIGS. 21A and 21), similar to the results with VSV loaded CAR T cells. In addition, a systemic boost with reovirus, but not with a heterologous virus (VSV), re-activated the virus specific CAR T cells leading to tumor cures in most mice. In vitro reovirus loading on EGFRvIII CAR T cells also increased the in vivo persistence/expansion of CAR T cells that could be detected at endpoint compared to unloaded CAR T (FIG. 21C). An additional systemic boost with reovirus (but not with VSV) induced further expansion of CAR T cell numbers demonstrating that this expanded population is reovirus specific.

A similar, fully systemic, regimen of CAR T cells pre-loaded with reovirus, with subsequent boosting with reovirus, also cured greater than 80 percent of mice of aggressive CT2A-EGFRvIII brain tumors (FIGS. 21D and 21E), indicating that this strategy is not dependent upon the location of the tumor being treated. The systemic boost with reovirus also greatly expanded the number of CAR T cells in the brains of tumor-cured mice compared to mice treated with CAR T cells alone, or with CAR T cells loaded with reovirus but not treated with a subsequent virus boost (FIG. 21F). Unlike the subcutaneous melanoma model (FIG. 21C), in the case of these intra-cranial tumors, CAR T cells loaded with reovirus but not boosted in vivo were not detected at higher frequency than unloaded CAR T cells alone (FIG. 21F), which may be explained by different properties of the subcutaneous and intra-cranial tumor locations. When re-stimulated in vitro, splenocytes from mice treated with reovirus-loaded CAR T and reovirus boost consistently secreted the highest levels of IFNγ upon co-culture with CT2AEGFRvIII cells (FIG. 21G). Moreover, splenocytes from mice that survived long term in various groups in the experiment of FIG. 21E secreted higher levels of IFNγ when re-stimulated in vitro with CAR target cells.

Taken together, these results demonstrate that co-administration of viruses (e.g., oncolytic viruses) with CAR T cells expands dual specific CAR T cells that can subsequently be re-activated in vivo by virus. The dual specific CAR T cells persist for long periods and are retained at high numbers both in the organs cleared of tumor (such as the brain) as well as in the spleen. These virus-boosted, dual specific CAR T cells have superior efficacy compared to virus alone, to CAR T without virus-mediated TCR stimulation, or to virus-loaded but not boosted CAR T cells. Moreover, these therapeutic effects were observed across tumor sites and with distinct viruses such as VSVs and reoviruses.

### Dual Specific (DS) CAR T Cells Target Virally Encoded MHC Restricted Tumor Antigens

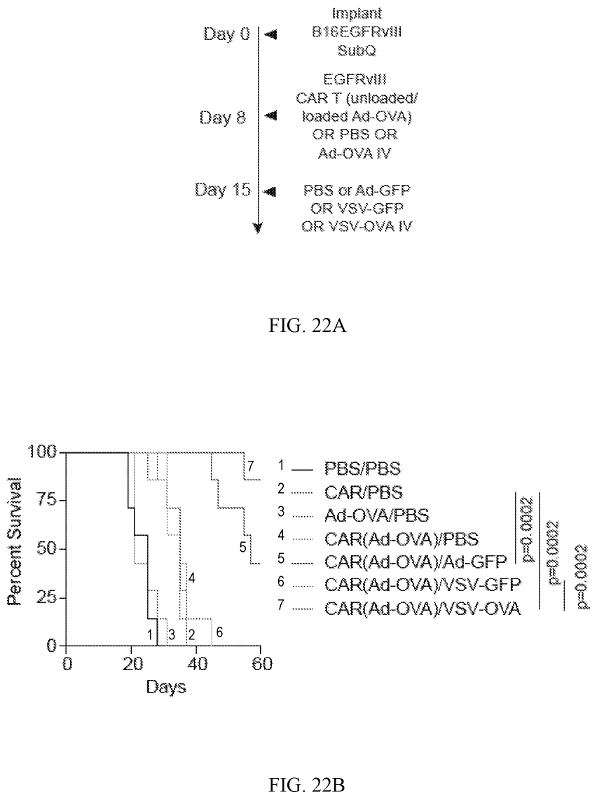

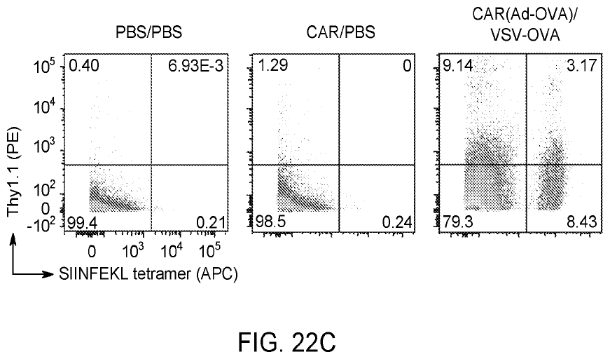

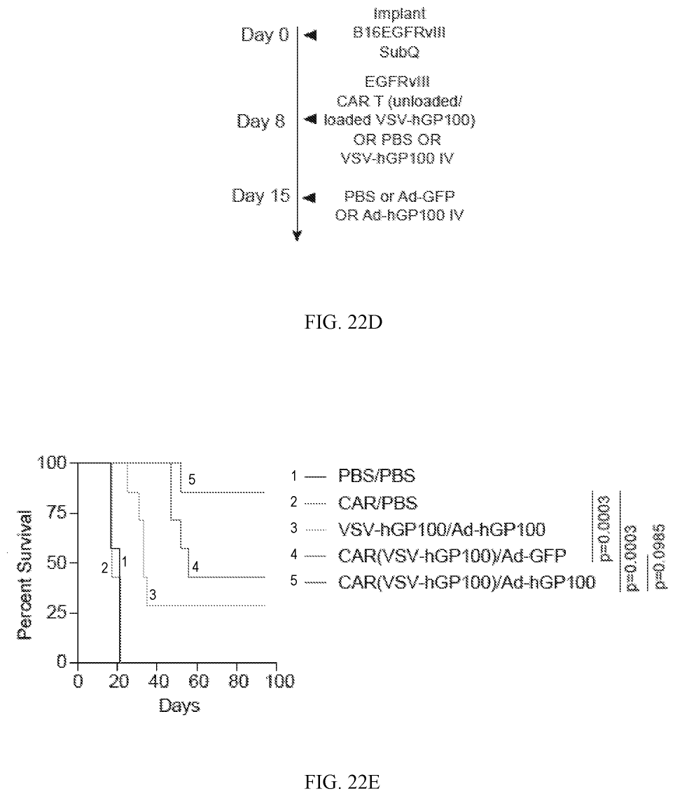

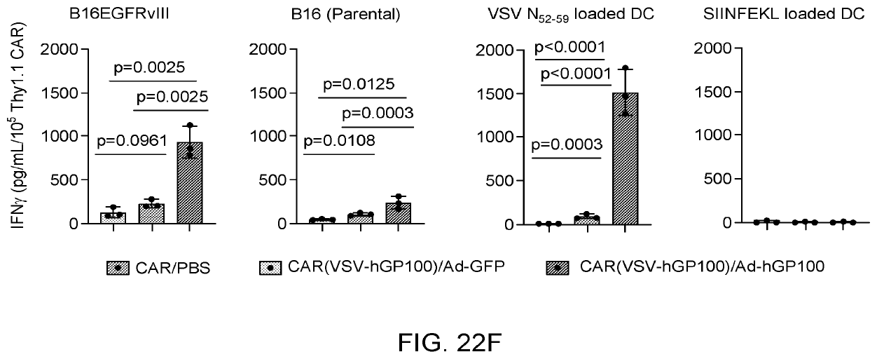

It was next examined whether dual specific CAR T cells could also be primed through their TCR against antigens encoded by viruses as well as against the viral epitopes themselves. Mice bearing B16EGFRvIII tumors were treated with CAR T cells loaded with a replication defective adenoviral vector encoding the ovalbumin (OVA) gene (FIG. 22A). Unlike with either VSV or reovirus, CAR T cell loading with the Ad-OVA vector did not enhance CAR T cell efficacy compared to unloaded CAR T cells (FIG. 22B), possibly due to a different ability of Ad vectors (which do not infect murine cells efficiently) to stimulate TLR signaling in murine CAR T cells. However, the therapeutic efficacy of CAR T cells loaded with Ad-OVA could still be significantly enhanced by systemic boosting with the homologous virus (Ad-GFP), as was the case with CAR T cells loaded with either VSV or reovirus (FIG. 22B). As with VSV and reovirus, the efficacy of CAR T cells loaded with Ad-OVA could not be enhanced by systemic boosting with a heterologous virus such as VSV-GFP (FIG. 22B). In contrast, Ad-OVA loaded CAR T cells could be re-activated in vivo very effectively by a heterologous virus expressing the OVA antigen, VSV-OVA (FIG. 22B).

Using tetramer staining, it was confirmed that ex vivo loading of the CAR T cells with Ad-OVA followed by a systemic boost with VSV-OVA generated a population of dual specific CAR T cells (˜3% of total CD8⁺ T cells in the spleen) (FIG. 22C). The expansion of OVA-specific CAR T was also associated with higher levels of expansion of SIINFEKL (SEQ ID NO:5) ignorant CAR T compared to mice treated with unloaded CAR T cells (FIG. 22C).

In the experiment of FIGS. 22A and 22B, OVA was not a tumor associated antigen (not expressed in the B16EGFRvIII tumors) and acted strictly as an added immunogen or antigenic composition for CAR T cell expansion and activation. Next, the efficacy of in vitro loading of CAR T cells with oncolytic VSV encoding the human gp100 (hGP100) melanoma antigen to raise T cell responses against the murine tumor associated GP100 antigen expressed in the B16EGFRvIII tumors was examined. Whereas treatment of mice bearing B16EGFRvIII tumors with CAR T cells alone was ineffective in the absence of pre-conditioning, as before, ex vivo loading with VSV-hGP100 significantly enhanced therapeutic efficacy (FIG. 22E), due to a combination of altered function of the CAR T cells themselves and the delivery of oncolytic VSV to the tumors. However, providing a further systemic boost with Ad-hGP100 to the loaded CAR T cell arm generated significant additional therapy and cured 7/8 mice of their tumors (FIG. 22E). At completion of the experiment (day 95), co-culture of CAR T cells isolated from spleens of mice treated with CAR(VSV-hGP100) and boosted with Ad-hgp100 revealed subpopulations that were functionally specific for both the CAR antigen and the MHC restricted VSV N₅₂₋₅₉ epitope, but not for the irrelevant OVA/SIINFEKL (SEQ ID NO:5) antigen (FIG. 22D). A small population of CAR T that secreted IFNγ was also observed when re-stimulated with B16 parental tumor cells, suggesting specificity for the MHC restricted GP100 antigen (FIG. 22D).

### Human Dual Specific CAR T Cells can be Expanded In Vitro and Possess Novel Functionality

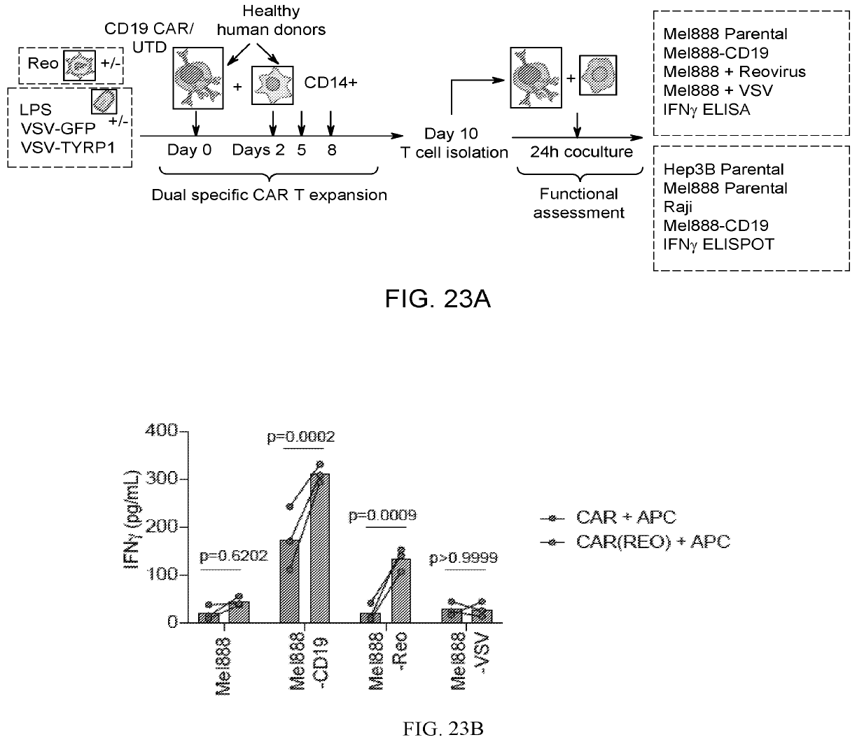

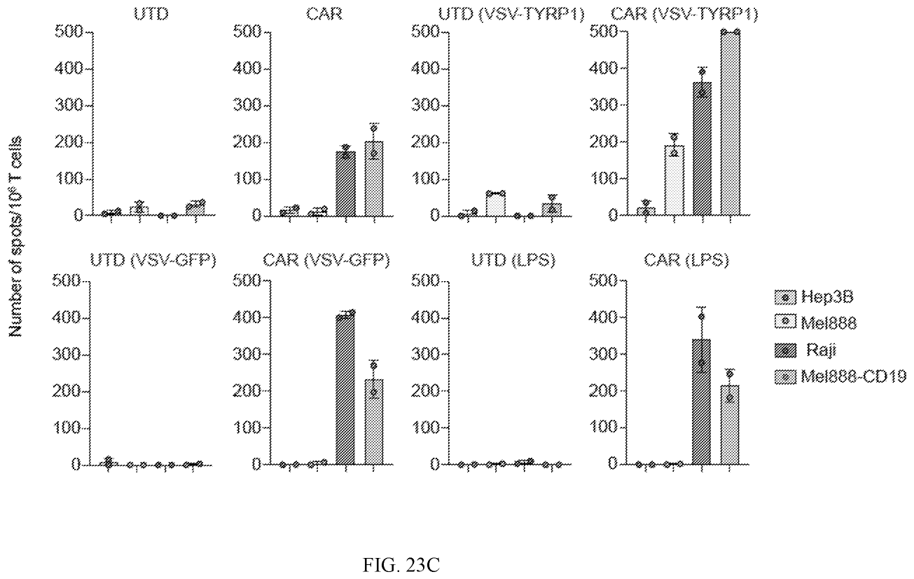

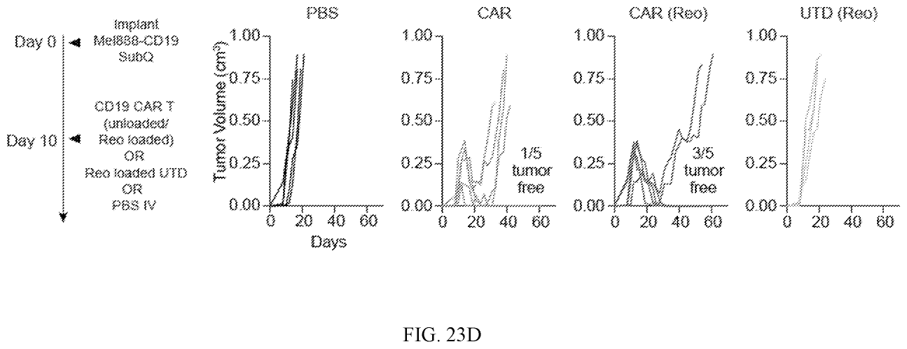

To investigate whether human virus specific cells could be expanded and functionally validated in vitro, CD19-specific CAR T cells were loaded with virus, and co-cultured with autologous monocytes as antigen presenting cells (APCs) to expand the virus specific T cell population. At the end of a 10-day priming phase, T cells were isolated and re-stimulated with CAR-, or TCR-, specific targets to test their functionality (FIG. 23A). Human CD19 CAR T cells expanded with unloaded APCs secreted IFNγ in response to Mel888 cells modified to express CD19, but not in response to parental tumor cells, or tumor cells pre-infected with reovirus or VSV (FIG. 23B). CAR T cells loaded with reovirus secreted significantly higher levels of IFNγ upon stimulation with CD19-expressing target cells than CAR T expanded without virus from the same donors. These reovirus loaded CAR T cells also recognized reovirus infected, but not VSV infected, targets (FIG. 23B). These results demonstrate that in vitro priming of the CAR T cells was possible by hand-off of the loaded virus to APCs for presentation of viral epitopes to the CAR T cells to generate dual specific CAR T with TCR specificity for reovirus.

Similarly, the expansion and functional re-activity of un-transduced T cells (UTD), or of CAR T cells loaded with VSV-GFP, VSV expressing the melanoma antigen tyrosinase related protein (TYRP1), or LPS were tested by co-culturing them with Hep3B (CD19⁻, TYRP1⁻), Mel888 (CD19⁻, TYRP1⁺), Raji (CD19⁺, TYRP1⁻), and Mel888 (CD19⁺, TYRP1⁺) using an IFNγ ELISPOT. CD19 CAR T cells produced a similar number of spots when co-cultured with either Raji or Mel888-CD19 cells (FIG. 23C). However, consistent with the murine data, human CD19 CAR T cells loaded with VSV-TYRP1 expressed greater levels of IFNγ when co-cultured with these same CAR target cells. Moreover, IFNγ reactivity was observed against Mel888 parental cells (which express the TYPR1 antigen) only from CAR T cells expanded with the VSV-TYRP1 virus. Loading the CAR T cells with a virus expressing the control GFP antigen improved the activity of the CAR T cells against CD19 target cells but did not prime the CAR T cells for dual specificity against Mel888 melanoma targets (FIG. 23C). Also consistent with the murine data, treating CAR T cells with LPS increased their activity against CD19 targets but did not confer any additional TCR specificity against melanoma targets. Taken together, these results demonstrate that antigenic composition loading (e.g., virus loading) leads to the priming of both mouse and human CAR T cells through their endogenous TCR against both viral antigens (FIGS. 23B and 23C) as well as against virus encoded MHC restricted antigens (FIG. 23C).

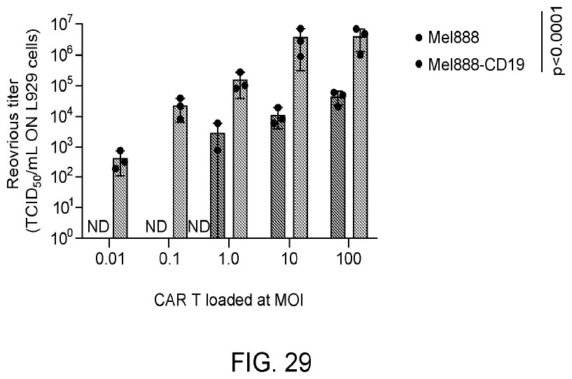

To maximize the use of viral carriage to tumors by ex vivo-loaded human CAR T cells, the optimal loading conditions by which virus could be made available to target tumor cells were examined. In vitro, virus-loaded anti-CD19 CAR T cells could transfer/release reovirus for infection of both CAR antigen positive and negative tumor cells (FIG. 29). However, levels of hand-off of the virus were consistently higher at any given MOI of loading when the target cells expressed the CAR antigen.

Finally, the therapeutic efficacy of reovirus loaded human CAR T cells against human Mel888-CD19 tumors in NSG mice was tested. Using the in vitro loading conditions defined to be optimal in FIG. 29 (MOI 10), CAR T cells loaded with reovirus were more effective than either unloaded CAR T cells or virus loaded, UTD T cells (FIG. 23F). NSG mice treated intravenously with the equivalent virus dose as was loaded onto the CAR T cells (10⁸ pfu/mouse) had to be euthanized by day 15 due to toxicity, showing that CAR T cell carriage of virus provides the additional benefits of both protecting systemically delivered virus and allowing for increased safety of delivery. Taken together with the murine immune competent studies, these results confirm that ex vivo virus loading significantly enhances the activity of CAR T cell therapy against solid tumors.

### Ex Vivo Virus Loading of CAR T Cells is Therapeutically Effective Even in Cases of Limited CAR Target Expression

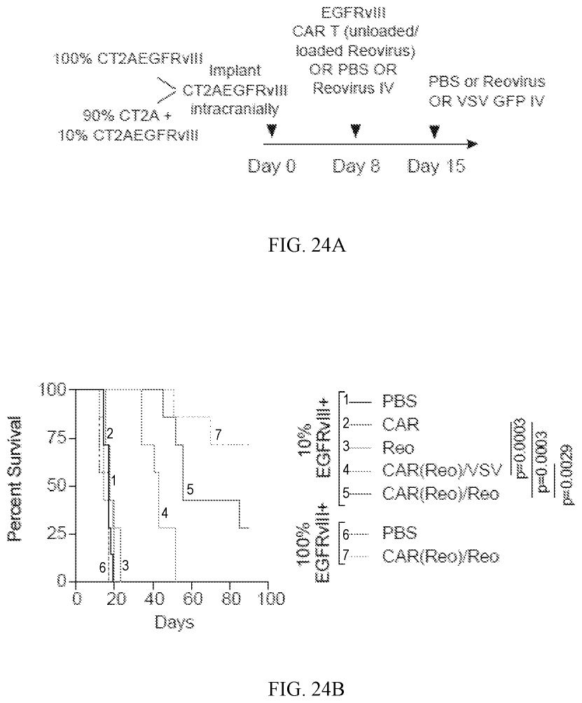

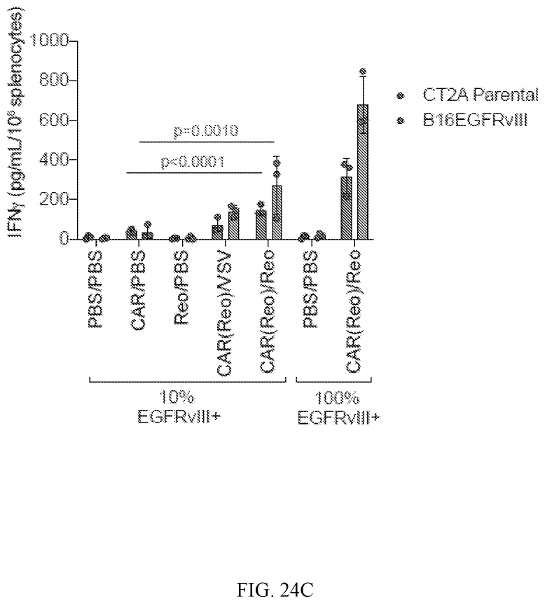

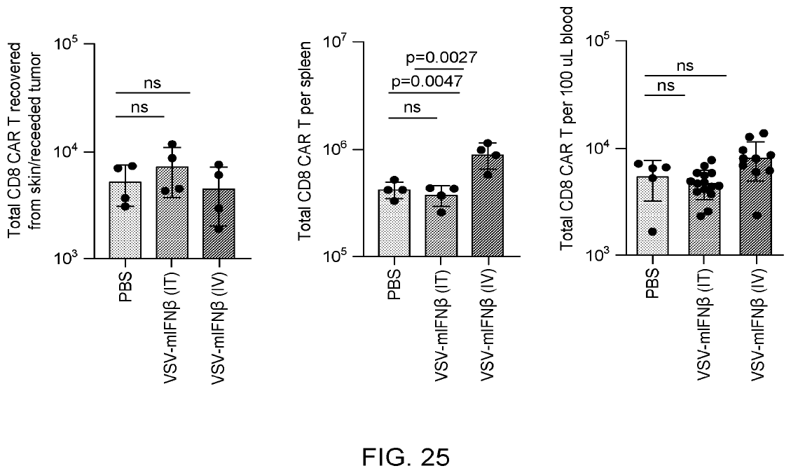

It was examined whether the combined cytolytic and inflammatory oncolytic activities of the both CAR T and oncolytic virus modalities would facilitate CAR T cell therapy even in tumors where CAR antigen expression was significantly limiting. Animals were generated to bear tumors composed entirely of target expressing cells (100% CT2AEGFRvIII) or a mixed population (10% CT2AEGFRvIII+90% CT2A). Consistent with this hypothesis, CAR T cell therapy against brainstem tumors consisting of 90% CT2A cells and 10% CT2A-EGFRvIII cells was completely ineffective, as was systemically delivered reovirus (FIG. 24B). Loading of the CAR T cells with reovirus significantly enhanced therapy but did not generate any cures consistent with both enhanced CAR T cell function and delivery of the oncolytic virus to the tumor. However, in vitro loading of the CAR T cells with reovirus combined with a further systemic boost with reovirus significantly improved therapy still further and led to cures in two of the seven mice (FIG. 24B).

The functionality of T cells at endpoint when restimulated with B16EGFRvIII cells or CT2A cells was also assessed. Splenocytes from mice treated with reovirus-loaded CAR T cells and a subsequent systemic boost with reovirus produced significant levels of IFNγ when re-stimulated with CT2A cells (FIG. 24C), indicating that the treatment induced epitope spreading leading to either endogenous T cell responses or dual specific CAR T cells reactive against the tumor. These splenocytes also contained the highest levels of CAR T cells that secreted IFNγ upon re-stimulation with B16EGFRvIII tumor cells (FIG. 24C). Comparatively, however, mice with tumors consisting entirely of CT2A-EGFRvIII cells treated with reovirus loaded CAR T cells combined with a further systemic boost with reovirus experienced significantly better therapy than did mice bearing 10% CT2AEGFRvIII tumors (FIG. 24B), and splenocytes from these mice showed improved responses against both endogenous CT2A-derived antigens as well as against the CAR antigen (FIG. 24C).

Taken together, these results demonstrate that CAR T cells with native TCR specificity to oncolytic virus antigens (e.g., VSV N or unspecified reovirus antigens), foreign irrelevant antigens (e.g., OVA), and tumor antigens (e.g., GP100 or TYRP1) were all expanded in vivo and generated improved therapeutic outcomes compared to CAR T cells, or oncolytic virus, alone across tumor types and locations. The increased efficacy was associated with higher levels of the oncolytic virus delivered to the lymph nodes and tumors, altered memory differentiation profiles, improved functionality of CAR T cells, and increased levels of epitope spreading in endogenous T cell populations.

These results demonstrate that CAR T cell therapy can be successfully combined with antigenic compositions generally and/or oncolytic virus therapy specifically using a fully systemic delivery regimen to activate the TCR and may allow patients to bypass lymphodepletion.

### Materials and Methods

### Experimental Design

These experiments were designed to evaluate the benefits of combining oncolytic viruses such as VSV and reovirus with CAR T cell therapy against cancer (e.g., solid tumors). The investigators were not blinded to the allocation of groups during experiments or subsequently during the analysis. Sample sizes were chosen on the basis of estimates from pilot experiments and other results. Animals were randomized to treatment groups following tumor implantation using the GraphPad QuickCalcs online tool (graphpad.com/quickcalcs/randMenu/). The n values and particular statistical methods are indicated in the figure descriptions as well as in the statistical analysis section.

### Cell Lines and Viruses.

B16 murine melanoma cells, CT2A murine glioma, BHK, L929, and 293T cells were originally obtained from ATCC and maintained in DMEM (HyClone) plus 10% FBS (Life Technologies). Cells were tested for Mycoplasma using the MycoAlert Mycoplasma Detection Kit (Lonza). The B16EGFRvIII cell line was generated by retroviral transduction of B16 cells with the pBABE PURO vector encoding the murine EGFRvIII modified by the deletion of 500 amino acids from the intracellular domain of the protein. A clonally derived cell line was subsequently maintained in 1.25 μg/mL of puromycin (Sigma). The CT2AEGFRvIII cell line was maintained in DMEM plus 10% FBS.

VSV expressing murine IFNβ or GFP was rescued from the pXN2 cDNA plasmid and propagated on BHK cells at low multiplicity of infection. 24 hours post infection, supernatant was harvested, filtered through a 0.22 μm filter to remove debris, and purified through a 10% sucrose cushion. Virus titers were determined by plaque assay on BHK cells. Wild-type Reovirus type 3 (Dearing strain) was obtained from Oncolytics Biotech (Calgary, AB, Canada), and stock titers were measured by plaque assay on L929 cells.

### Mice

Female C57BL/6 (stock 000664) mice and 4-week-old NSG mice (stock 005557) were obtained from The Jackson Laboratory. All mice were obtained at 4-8 weeks of age and maintained in a specific pathogen-free BSL2 biohazard facility. Experimental mice were co-housed and exposed to a 12:12 hour light-dark cycle with unrestricted access to water and food. The ambient temperature was restricted to 68 to 79° F., and the room humidity ranged from 30-70%.

### Murine CAR T Cell Preparation

The EGFRvIII third generation MSGV1 retroviral CAR construct contains the CD28, 4-1BB, and CD3z moieties, in tandem with the scFv derived from the human monoclonal antibody 139, and the marker Thy1.1. CAR T cells were prepared as described elsewhere (Riccione et al., J Vis. Exp., 96:52397 (2015)). Briefly, splenocytes were isolated from donor C57BL/6 mice were made into a single cell suspension and cultured in RPMI (HyClone) supplemented with 10% FBS, 50 μM 2-Mercaptoethanol (Sigma), 1% PenStrep (Corning), 1% NEAA (Corning), 1% Sodium Pyruvate (Corning), 50 U/mL human IL2 (Novartis), and 2.5 μg/mL Concanavalin A (Sigma). Retroviral supernatant was produced from 293T cells co-transfected with the MSGV1 retroviral plasmid and the helper plasmid pCL Eco (Imgenex), and T cells were transduced on RetroNectin-coated plates (Takara) 2 days after stimulation. Cells were split one day after transduction and used for in vitro analysis or in vivo administration on day 4 or 5. Transduced cells were identified by the expression of Thy1.1.

### Human CAR T Cell Preparation

Peripheral blood mononuclear cells (PBMCs) were from healthy donor apheresis cones. Cells were isolated using Lympholyte-H density separation (Cedarlane) and cultured in AimV Media (ThermoFisher) supplemented with 5% human AB serum (Sigma) and 1% PenStrep and stimulated with 100 U/mL of human IL2 and 50 ng/mL anti-CD3 OKT3 antibody (Biolegend #317326). 48 and 72 hours later, cells were transduced twice with lentiviral supernatant collected from 293T producer cells co-transfected with the anti-CD19 CAR and R8.91QV and pMD.G packaging plasmids, on RetroNectin-coated plates (Takara). Cells were split every two days and collected 4 days after the second transduction for in vitro and in vivo experiments.

### In Vivo Studies

Mice were challenged subcutaneously with 2×10⁵ B16EGFRvIII cells in 100 μL PBS (HyClone). Subcutaneous tumors were treated with VSV-mIFND, VSV-GFP, reovirus, Ad-OVA, or Ad-GFP delivered intratumorally in 50 μL of PBS or intravenously in 100 μL of PBS. Later IV doses consisted of 5×10⁷ pfu of virus in 50 μL PBS. CAR T cells were delivered intravenously by tail vein injection in 100 μL of PBS. Tumors were measured using calipers 3 times per week, and mice were euthanized using CO₂ when tumors reached 1.0 cm in diameter. For experiments requiring radiation, mice were subjected to 5 Gy TBI 24 hours before CAR T cell therapy. The survival end point was reached when the tumor size reached 1 cm in diameter. Tumor volume was calculated as follows:

$$Volume = \frac{( Length \times {Width}^{2} )}{2} .$$

CT2AEGFRvIII tumor cells were stereo-tactically implanted into the brainstem of C57Bl/6 mice as described elsewhere (Caretti et al., Brain Pathol., 21:441-451 (2011)). Mice were monitored daily for gross neurologic symptoms including gait abnormalities, hunching, lethargy, seizures, paralysis, circling, and head tilt. Upon presentation of gross neurologic symptoms or poor body conditioning, mice were euthanized.

### In Vitro Loading of CAR T Cells

CAR T cells were prepared as described above. On day 4 or 5 following transduction, cells were pelleted and washed twice in PBS. Pelleted cells were then incubated for 60 minutes at 4° C. with virus stock at a MOI of either 1 or 10. CAR T cell/virus pellets were then washed 2-3 times with PBS and resuspended at the appropriate cell density for in vivo administration.

In Vitro Sorting of CAR T Cells from Spleens

Single-cell suspensions from mouse spleens were prepared, and Thy1.1 (CD90.1) CAR T cells were isolated using CD90.1 MicroBeads (Miltenyi Biotech, order no. 130-121-273), as directed by the manufacturer prior to being used in in vitro re-stimulation assays.

### Generation of Murine Bone Marrow Dendritic Cells (BMDCs)

Femurs were collected from C57/B16 mice and bone marrow flushed into RPMI media using a 25 g needle. Bone marrow was treated with ACK lysis buffer, washed with serum free RMPI, and then resuspended in RPMI supplemented with 10% FBS plus 1× Penicillin/Strepomycin plus 50 μM PME supplemented with 20 ng/mL murine GMCSF (Peprotech). Cells were seeded at 10⁶ cells/well in 2 mL of a 24 well plate. Media was replaced with fresh mGMSF-containing media on day 3. BMDCs were collected on day 5.

### In Vitro Re-Stimulation Assays

CD8⁺ T cells (endogenous or Thy1.1⁺ CD8⁺ CAR T cells) isolated from mouse spleens were co-cultured with target tumor cells (B16EGFRvIII or B16) pre-treated for 24 hours with IFNγ, or with murine in vitro matured dendritic cells pre-loaded for 24 hours with VSV-N₅₂₋₅₉ or OVA-derived SIINFEKL (SEQ ID NO:5) peptides (5 μg/well) at an E:T ratio of 10:1. 48 hours later, levels of IFNγ were measured by ELISA (Mouse IFNγ ELISA Kit; OptEIA, BD Biosciences).

Virus-Loaded Human CAR T Cell Co-Culture with CD14+ Dendritic Cells

Fresh PBMCs from healthy donors were acquired to make human CAR T cells as described above. Autologous monocyte-derived dendritic cells were matured by isolating CD14⁺ cells by magnetic sorting (Miltenyi Biotech), followed by incubation with human GM-CSF (800 U/mL) and IL-4 (1000 U/mL). On Days 3 and 5, media was replaced with human GM-CSF (1600 U/mL) and IL-4 (1000 U/mL). On Day 7, non-adherent cells were collected, washed with PBS, and resuspended in medium containing GM-CSF (800 U/mL), IL-4 (1000 U/mL), TNFα (1100 U/mL), IL-10 (1870 U/mL), IL-6 (1000 U/mL), and PGE2 (1 μg/mL). Two days later, dendritic cells were harvested for co-incubation with activated CAR T cells loaded in vitro with virus (as described above) at a ratio of 1:1. After 10 days of 25 co-culture of dendritic cells (DCs) with CAR T cells, CD3⁺ T cells were re-isolated using a magnetic sorting kit (Miltenyi Biotech) and were immediately co-cultured with IFNγ pre-treated (200 U/mL for 12 hours) tumor cell targets (parental Mel888; Mel888 stably transfected with human CD19; or reovirus infected Mel888 (MOI 0.1) or VSV infected Mel888 cells (MOI 0.001)) at an E:T ratio of 10:1, and 24 hours later, levels of IFNγ were 30 assayed by ELISA (R&D). Alternatively, on day 10 post co-culture of virus-loaded CAR T cells with dendritic cells, CD3⁺ T cells were re-isolated by magnetic bead sorting, and 10⁶ T cells were co-cultured with IFNγ pretreated parental Hep3B, Mel888 cells, Raji, or Mel888-CD19 cells at an E:T ratio of 10:1 in ELISPOT wells (R&D Human IFN-gamma ELISpot kit, EL285). 48 hours later, wells were developed, and the number of spots counted.

### Flow Cytometry

Flow cytometry was performed on cultured cells or freshly explanted spleens, blood, or tumors, or where no palpable tumor existed, a 1.5×1.5 cm area of skin. Tumors and skin were weighed and treated with Liberase TL (Roche) and DNAse I (Sigma) for 30-45 minutes at 37° C. Up to 30 mg of tumor or other tissue was stained and run on the flow cytometer. 100 μL of blood collected by submandibular vein bleed was subjected to red blood cell lysis and stained. Samples were fixed in 4% formaldehyde and analyzed using the BD FACSCantoX SORP flow cytometer using FACSDiva v8.0 software or the ZE5 Cell Analyzer using Everest v2.0 software. Data was analyzed using FlowJo version 10.5.

Mouse cells were stained with fluorochrome-conjugated antibodies against combinations of the following antigens: CD80 (Biolegend #140410/140416, clone 53-5.8, dilution 1:500), CD4 (Biolegend #100451 clone GK1.5), Thy1.1 (Biolegend #202524, clone OX-7, dilution 1:1000; or eBioscience #11-0900-85, cloneH1S51, dilution 1:500), CD45 (Biolegend #103114, clone 30-F11, dilution 1:500), CD45.2 (Biolegend #109828, clone 104, dilution 1:250), CD127 (Biolegend #135007 clone A7R34 dilution 1:00), CD62L (Biolegend #104431, clone MEL-14, dilution 1:200), and KLRG1 (Biolegend #138411/138419 clone 2F1/KLRG1 dilution 1:200). TCR Vβ analysis was performed using the Anti-Mouse TCR Vβ Screening Panel (BD Pharmingen #557004, dilution 1:5).

Cells were stained with the H-2K(b) VSV NP₅₂₋₅₉ RGYVYQGL (SEQ ID NO:6; Brilliant Violet 421-labeled Tetramer) at a dilution of 1:500 or the H-2K(b) chicken ova257-264 SIINFEKL (SEQ ID NO:5; APC-labeled Tetramer) at a dilution of 1:150, which were obtained from the NIH Tetramer Core Facility.

Intracellular staining was performed on cells stimulated for 5 hours in the presence of monensin and brefeldin A (dilution 1:1000, BD) and CD107a (Biolegend #121625, clone 1D4B. 1:150). Intracellular cytokines were detected using the following fluorochrome-conjugated antibodies: IFNγ (Biolegend #505806 clone XMG1.2, dilution 1:200), TNFα (Biolegend #506329 clone MP6-XT22, dilution 1:200), and IL2 (Biolegend #503808 clone JES6-5H4, dilution 1:200). The tumor cell killing assay was performed using target and non-target cells stained with Cell Trace Violet or CFSE (Thermo Fisher). Cell viability was determined using the Zombie fixable live dead viability dye (Biolegend #423106, dilution 1:1500).

### Statistical Analyses

Data processing was performed in Microsoft Excel. Graphing and statistical analysis was performed with GraphPad Prism 9 software (Graphpad). Single comparisons were made using unpaired two tailed T tests. Multiple comparisons were analyzed using one-way or two-way ANOVAs with a Tukey's or Sidak post-hoc multiple comparisons test. Survival data was assessed using the Log-Rank Mantle Cox test with a Bonferonni multiple comparisons correction. Group data is expressed as group mean±SD.

### Example 6: Treating Cancer with CAR⁺ T Cells and Virus

CAR⁺ T cells expressing a CAR⁺ that can target (e.g., target and bind) a cancer antigen and viruses (e.g., oncolytic viruses) are systemically administered to mammals (e.g., mice or humans) having cancer. In some experiments, CAR⁺ T cells and viruses are in separate compositions that are co-administered. In some experiments, CAR⁺ T cells can be loaded (e.g., coated) with viruses and administered together as a single composition.

A boost (e.g., subsequent administration) of virus is provided systemically to mammals about 1 week after the co-administration of CAR⁺ T cells and viruses. Control mammals are boosted with PBS.

CAR⁺ T cells and viruses delivered to a mammal can generate dual-specific, T_{RM} T cells that are CAR⁺ and include an endogenous TCR specific for an antigen of the virus.

The systemic boost with virus can re-activate the dual-specific, T_{RM} CAR⁺ T cells in vivo via the endogenous TCR specific for an antigen of the virus. Those re-activated dual-specific, T_{RM} CAR⁺ T cells can target (e.g., target and destroy) cells (e.g., cancer cells) presenting antigens recognized by the CAR present on the CAR⁺ T cells and/or can generate effector T cells that are CAR⁺ and that can target (e.g., target and destroy) cells (e.g., cancer cells) presenting antigens recognized by the CAR present on those CAR⁺ effector T cells.

### Example 7: Treating Cancer with CAR⁺ T Cells and Virus Expressing Cancer Antigen

CAR⁺ T cells expressing a CAR that can target (e.g., target and bind) a cancer antigen and viruses expressing an antigen of interest (e.g., oncolytic viruses expressing an antigen) are systemically administered to mammals (e.g., mice or humans) having cancer. In some experiments, CAR⁺ T cells and viruses are in separate compositions that are co-administered. In some experiments, CAR⁺ T cells can be loaded (e.g., coated) with viruses expressing an antigen and administered together as a single composition.

A boost (e.g., subsequent administration) of a composition that includes the antigen of interest that was expressed by the viruses and that lacks the viruses is provided systemically to mammals about 1 week after the co-administration of CAR⁺ T cells and viruses. Control mammals are boosted with PBS.

CAR⁺ T cells and viruses expressing an antigen of interest delivered to a mammal can generate dual-specific, T_{RM} T cells that are CAR⁺ and include an endogenous TCR specific for the antigen of interest.

The systemic boost with the composition that includes the antigen of interest and that lacks the viruses can re-activate the dual-specific, T_{RM} CAR⁺ T cells in vivo via the endogenous TCR specific for the antigen of interest. Those re-activated dual-specific, T_{RM} CAR⁺ T cells can target (e.g., target and destroy) cells (e.g., cancer cells) presenting antigens recognized by the CAR present on the CAR⁺ T cells and/or can generate effector T cells that are CAR⁺ and that can target (e.g., target and destroy) cells (e.g., cancer cells) presenting antigens recognized by the CAR present on those CAR⁺ effector T cells.

### Example 8: Treating Cancer with CAR⁺ T Cells and Cancer Antigen

CAR⁺ T cells expressing a CAR that can target (e.g., target and bind) a cancer antigen and an antigen are systemically administered to mammals (e.g., mice or humans) having cancer. In some experiments, CAR⁺ T cells and antigens are in separate compositions that are co-administered. In some experiments, CAR⁺ T cells can be loaded (e.g., coated) with the antigens and administered together as a single composition.

A boost (e.g., subsequent administration) of antigens is provided systemically to mammals about 1 week after the co-administration of CAR⁺ T cells and the antigens. Control mammals are boosted with PBS.

CAR⁺ T cells and antigens delivered to a mammal can generate dual-specific, T_{RM} T cells that are CAR⁺ and include an endogenous TCR specific for the antigens.

The systemic boost with the antigens can re-activate the dual-specific, T_{RM} CAR⁺ T cells in vivo via the endogenous TCR specific for the antigens. Those re-activated dual-specific, T_{RM} CAR⁺ T cells can target (e.g., target and destroy) cells (e.g., cancer cells) presenting antigens recognized by the CAR present on the CAR⁺ T cells and/or can generate effector T cells that are CAR⁺ and that can target (e.g., target and destroy) cells (e.g., cancer cells) presenting antigens recognized by the CAR present on those CAR⁺ effector T cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises:
(a) administering a population of T cells with different endogenous T cell receptors (TCRs) to said mammal, wherein said T cells comprise a chimeric antigen receptor (CAR) that targets said cancer;
(b) administering a first antigenic composition to said mammal, wherein at least some of said T cells of said population form memory T cells within said mammal, wherein said memory T cells comprise said CAR and an endogenous TCR specific for an antigen of said first antigenic composition; and
(c) administering a second antigenic composition comprising said antigen to said mammal, wherein said memory T cells are stimulated via their endogenous TCRs to form effector T cells comprising said CAR, and wherein said effector T cells reduce the number of cancer cells within said mammal.

2. The method of claim 1, wherein said mammal is a human.

3. The method of claim 1, wherein said cancer is selected from the group consisting of brain stem gliomas, pancreatic cancers, bile duct cancers, lung cancers, skin cancers, prostate cancers, breast cancers, ovarian cancers, liver cancers, colorectal cancers, germ cell tumors, hepatocellular carcinoma, bowel cancers, multiple myeloma, lymphomas, and leukemias.

4. The method of claim 1, wherein said population of T cells with different endogenous TCRs comprises naïve T cells.

5. (canceled)

6. The method of claim 1, wherein said CAR can target a tumor-specific antigen on said cancer.

7. (canceled)

8. The method of claim 1, wherein said first antigenic composition comprises a virus.

9. The method of claim 8, wherein said virus is an oncolytic virus.

10-11. (canceled)

12. The method of claim 1, wherein said first antigenic composition comprises a virus expressing an antigen exogenous to said virus.

13. (canceled)

14. The method of claim 1, wherein said first antigenic composition comprises an antigenic polypeptide.

15-20. (canceled)

21. The method of claim 1, wherein said second antigenic composition is administered to said mammal at least 5 days after said administering of said population of T cells and said administering of said first antigenic composition.

22-24.

25. A method for generating memory T cells within a mammal, wherein said method comprises:
(a) administering a population of T cells with different endogenous T cell receptors (TCRs) to said mammal, wherein said T cells comprise a chimeric antigen receptor (CAR); and
(b) administering an antigenic composition to said mammal, wherein at least some of said T cells of said population form memory T cells within said mammal, wherein said memory T cells comprise said CAR and an endogenous TCR specific for an antigen of said antigenic composition.

26. The method of claim 25, wherein said mammal is a human.

27. (canceled)

28. The method of claim 25, wherein said CAR targets a tumor-specific antigen.

29. (canceled)

30. The method of claim 25, wherein said antigenic composition comprises a virus.

31. The method of claim 30, wherein said virus is an oncolytic virus.

32. The method of claim 31, wherein said virus is selected from group consisting of a vesiculovirus, a Maraba virus, a reovirus, adenoviruses, vaccinia viruses, Newcastle disease viruses, polioviruses, HSV viruses, and measles viruses.

33. (canceled)

34. The method of claim 25, wherein said antigenic composition comprises a virus expressing an antigen exogenous to said virus.

35. (canceled)

36. The method of claim 25, wherein said antigenic composition comprises an antigenic polypeptide.

37. (canceled)

38. The method of claim 25, wherein said mammal has cancer.

39. The method of claim 38, wherein said cancer is selected from the group consisting of brain stem gliomas, pancreatic cancers, bile duct cancers, lung cancers, skin cancers, prostate cancers, breast cancers, ovarian cancers, liver cancers, colorectal cancers, germ cell tumors, hepatocellular carcinoma, bowel cancers, multiple myeloma, lymphomas, and leukemias.

40-41. (canceled)


SEQUENCE LISTING (6 sequences)

SEQ ID NO: 1 - protein, 542 aa, Artificial
  EGFRvIII CAR
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRVDAAAT

SEQ ID NO: 2 - protein, 488 aa, Artificial
  GD2 CAR with CD28 component
MVLLVTSLLLCELPHPQVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKRAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 3 - protein, 492 aa, Artificial
  GD2 CAR with 4-1BB component
MVLLVTSLLLCELPHPQVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKRAAATTTKPVLRTPSPVHPTGTSQPQRPEDCRPRGSVKGTGLDFACDIYIWAPLAGICVALLLSLIITLICYHRSRKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 4 - protein, 476 aa, Artificial
  CD19 CAR
PDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAHYYYGGSYAMDYWGQGTSVTVSSAAATTTKPVLRTPSPVHPTGTSQPQRPEDCRPRGSVKGTGLDFACDIYIWAPLAGICVALLLSLIITLICYHRSRNSRRNRLLQVTTMNMTPRRPGLTRKPYQPYAPARDFAAYRPKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 5 - protein, 8 aa, Artificial
  ovalbumin peptide
SIINFEKL

SEQ ID NO: 6 - protein, 8 aa, Artificial
  H-2K(b) VSV peptide
RGYVYQGL